(19) United States
(12) Patent Application Publication
(10) Pub. No.: US 2022/0339144 A1
(43) Pub. Date: Oct. 27, 2022

(54) PYRIDINYL INDOLE COMPOUNDS AND METHODS OF USE THEREOF

(71) Applicant: Chinook Therapeutics Canada, Inc., Vancouver (CA)

(72) Inventors: Jinyue DING, Burnaby (CA); Robert GOMEZ, North Vancouver (CA); James Gifford GUTHRIE, Vancouver (CA); David Andrew POWELL, Vancouver (CA); Tao SHENG, Coquitlam (CA)

(21) Appl. No.: 17/714,962

(22) Filed: Apr. 6, 2022

Related U.S. Application Data
(60) Provisional application No. 63/172,551, filed on Apr. 8, 2021.

(51) Int. Cl. A61K 31/4439; C07D 491/107; C07D 498/08; C07D 401/14; C07D 487/10; C07D 495/10; C07D 491/048; C07D 487/04; A61K 31/537; A61K 31/553; A61K 31/444; A61K 31/55; A61K 45/06

(52) U.S. Cl. CPC A61K 31/4439; C07D 491/107; C07D 498/08; C07D 401/14; C07D 487/10; C07D 495/10; C07D 491/048; C07D 487/04; A61K 31/537; A61K 31/553; A61K 31/444; A61K 31/55; A61K 45/06

(57) ABSTRACT

Provided herein are compounds for the treatment or prevention of AMPK-(5′ adenosine monophosphate-activated protein kinase) mediated diseases. Also provided are pharmaceutical compositions comprising the compounds and methods of using the compounds and compositions.

### INCORPORATION BY REFERENCE TO ANY PRIORITY APPLICATION

The present application claims the benefit of priority to U.S. Provisional Application No. 63/172,551, filed Apr. 8, 2021, which is incorporated by reference in its entirety.

### FIELD OF THE INVENTION

Provided herein are pyridinyl indole compounds, which in certain embodiments, are modulators of AMPK (5′ adenosine monophosphate-activated protein kinase). Also provided are compositions comprising the compounds and methods of use thereof. The compounds provided are for use in the treatment, prevention or amelioration of an AMPK-mediated disease, condition or disorder.

### BACKGROUND OF THE INVENTION

AMPK (5′ adenosine monophosphate-activated protein kinase) is a key cellular sensor and regulator of energy metabolism and is proposed to play a central role in the pathogenesis of diseases such as obesity, metabolic syndrome, diabetes, inflammation, cancer, and chronic kidney diseases such as autosomal dominant polycystic kidney disease (ADPKD) (reviewed in Song, Cellular Signaling 2020 73: 1-12) and diabetic nephropathy (Salatto, Journal Pharm. and Experimental Therapeutics 2017 361: 303-311). Each AMPK molecule exists as a heterotrimeric complex composed of a catalytic a subunit and regulatory β and γ subunits. Multiple isoforms (α1/α2, β1/β2, γ1/γ2/γ3) encoded by different genes (PRKAA1/2, PRKAB1/2, PRKAG1/2/3) give rise to 12 possible distinct combinations each with distinct cellular and tissue expression profiles. AMPK is highly expressed in the kidney and transcriptomic analyses show strong mRNA expression of PRKAA2, PRKAB1 and PRKAG1 (encoding AMPK α2, β1 and γ1) in normal mouse and human kidneys (Song, Hum. Mol. Genet. 2009 18(13): 2328-2343; Park, Science 2018 360(6930): 758-763).

AMPK is activated in response to increased AMP:ATP and ADP:ATP ratios under conditions such as metabolic stress, hypoglycemia, hypoxia, ischemia, caloric restriction and exercise. Activated AMPK promotes the phosphorylation of key metabolic substrates including acetyl-CoA carboxylase (ACC), PGC 1 a, raptor and TSC2, which are linked to nearly all branches of cellular metabolism. AMPK exerts its pleiotropic effects through various AMPK substrates. The downstream consequences of AMPK activation include inhibition of mTORC1 activity (Inoki, Cell 2003 115(5): 577-590; Gwin, Mol. Cell 2008 30(2): 214-226), negative regulation of lipid metabolism (Carling, FEBS Lett 1987 223(2): 217-222), reduction of tissue inflammation and fibrosis (reviewed in Steinberg, Nat. Rev. Drug Discov. 2019 18(7): 527-551), reduction of oxidative stress (Salatto, Journal Pharm and Experimental Therapeutics 2017 361: 303-311), and regulation of multiple kidney transport proteins including direct inhibition of CFTR (Hallows, J. Clin. Invest. 2000 105(12): 1711-1721). Notably, all of the above AMPK-mediated mechanisms are implicated in the progression of ADPKD and/or diabetic nephropathy.

Chronic kidney disease (CKD) is a heterogeneous group of disorders affecting nearly 10% of the world's population (Wang, Semin. Nephrol. 2016 36:319-330), often leading to impaired renal function, renal failure, and ultimately end stage renal disease requiring dialysis and transplant. Despite the prevalence of CKD, very few new drugs have been approved in the past twenty years and most fail to address the underlying pathogenesis of these diseases but rather rely on blood pressure control with angiotensin-converting enzyme inhibitors and/or angiotensin II receptor blockers. However, some recent advances in specific kidney diseases include the approval of the vasopressin receptor 2 antagonist tolvaptan for ADPKD (Blair, Drugs 2019 79(3): 303-313) and the sodium-glucose co-transporter 2 (SGLT2) inhibitors empagliflozin and dapagliflozin for diabetic nephropathy (Neuen, Lancet 2019 7(11): 845-854). While these strategies slow disease progression in ADPKD and diabetic nephropathy, respectively, there remains clear unmet medical need to avoid kidney function decline and end stage renal disease in these patients.

Data from several preclinical studies support a key role for AMPK in kidney diseases. For example, salsalate (a prodrug dimer of salicylate) non-selectively activates AMPK through a direct interaction with the Ser108 residue of the AMPK β1 isoform and was found to attenuate cystic disease severity in an orthologous Pkd1 knockout mouse model of ADPKD (Leonhard, EBioMedicine 2019 47: 436-445). Metformin, a widely used drug for type II diabetic mellitus, is an indirect activator of AMPK reported to attenuate cystic kidney disease in orthologous mouse (Takiar, Proc. Natl. Acad. Sci. USA 2011 108(6): 2462-2467) and miniature pig (Lian, Br. J. Pharmacol. 2019 176(5): 711-724) models of ADPKD. It has yet to be reported in the literature, but it is also possible that AMPK activation could improve liver cysts often associated with ADPKD. Metformin was also reported to reduce high fat diet-induced metabolic derangement and renal injury in mice (Kim, Int. J. Mol. Med. 2013 32(6): 1293-302). A pan-selective AMPK activator, MK-8722, was renal protective in a ZSF1 progressive rat model of diabetic nephropathy and caused significant improvements in proteinuria, glomerular filtration rate, and kidney fibrosis (Zhou, J. Pharmacol. Exp. Ther. 2019 371(1): 45-55). Furthermore, selective activation of AMPK b1-containing isoforms with compounds such as PF-06409577 reduced proteinuria in the ZSF1 rat model of diabetic nephropathy (Salatto, Journal Pharm and Experimental Therapeutics 2017 361:303-311). Notably, the pan-AMPK activator MK-8722 caused significant cardiac hypertrophy and increased cardiac glycogen levels following 14 days of treatment in Wistar Han rats (Myers, Science 2017 357: 507-511). However, since the b2 subunit is the predominant form in the heart, it is hypothesized that b1-selective AMPK activators will not cause cardiac hypertrophy. Taken together, these data support the potential therapeutic benefit of AMPK activation, in particular the bl-containing isoform, for the treatment of chronic kidney diseases including ADPKD and diabetic nephropathy. There remains a need for the identification of small molecules AMPK activators, particularly compounds useful for the treatment of AMPK-mediated diseases.

### SUMMARY OF THE INVENTION

In certain embodiments, provided herein are compounds of Formula (I) or a pharmaceutically acceptable salt, hydrate or solvate thereof. In certain embodiments, the compounds are AMPK activators. In certain embodiments, the compounds are activators of the bl-containing isoform of AMPK. In certain embodiments, the compounds provided herein will confer therapeutic benefits associated with the activation of AMPK, including treating or preventing polycystic kidney disease, chronic kidney disease (CKD), end stage renal disease (ESRD), diabetic nephropathy, acute kidney injury and polycystic liver disease.

In certain embodiments, provided herein are compounds of Formula (I):

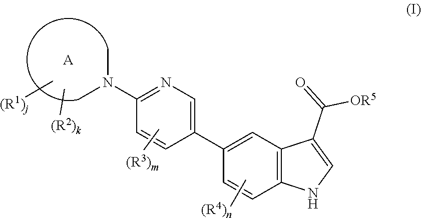

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹ , —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
each R⁴ is independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

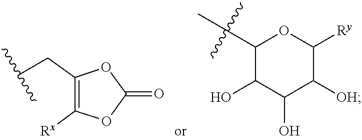

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
   (i) R⁷ and R⁸ are each independently hydrogen, alkyl, haloalkyl or alkoxyalkyl; or
   (ii) R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3;
m is 1, 2 or 3;
n is 1 or 2; and
each t is independently 0, 1 or 2.

Also provided are pharmaceutical compositions formulated for administration by an appropriate route and means containing therapeutically effective concentrations of one or more of the compounds provided herein, or pharmaceutically acceptable salts, hydrates or solvates thereof, and optionally comprising at least one pharmaceutical carrier.

In another aspect, provided herein are methods of treating a disease or disorder mediated by AMPK, comprising administering to a subject having such disease or disorder, a therapeutically effective amount of one or more compounds disclosed herein, or a pharmaceutically acceptable salt, hydrate or solvate thereof, or the pharmaceutical compositions disclosed herein. In certain embodiments, the disease or disorder is polycystic kidney disease, autosomal dominant polycystic kidney disease (ADPKD), autosomal recessive polycystic kidney disease (ARPKD), chronic kidney disease (CKD), end stage renal disease (ESRD), diabetic nephropathy, acute kidney injury or polycystic liver disease. In yet certain embodiments, the disease or disorder is associated with the mutation of PKD1, PKD2, PKHD1 or DZIP1L, or a combination of mutations thereof.

Also provided herein are combination therapies using one or more compounds or compositions provided herein, in combination with other pharmaceutical agents for the treatment of the diseases and disorders described herein. These and other aspects of the subject matter described herein will become evident upon reference to the following detailed description and drawings.

### DETAILED DESCRIPTION OF THE INVENTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry terms may be found in reference works, including but not limited to, Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

As used herein, C₁-Cₓ includes C-₁-C₂, C₁-C₃ . . . C₁-Cₓ. C₁-Cₓ refers to the number of carbon atoms that make up the moiety to which it designates (excluding optional substituents).

The term “alkyl” as used herein and unless otherwise indicated, refers to a saturated hydrocarbon chain that may be a straight chain or branched chain, containing the indicated number of carbon atoms or otherwise having from one to ten, one to eight, one to six or one to four carbon atoms, and which is attached to the rest of the molecule by a single bond. In certain embodiments, the hydrocarbon chain is optionally deuterated. For example, C₁-C₃alkyl indicates that the group may have from 1 to 3 (inclusive) carbon atoms; C₁-C₆ alkyl indicates that the group may have from 1 to 6 (inclusive) carbon atoms. In some embodiments, an alkyl is a C₁-C₃alkyl which represents a straight-chain or branched saturated monovalent hydrocarbon group having 1 to 3 carbon atoms. In some embodiments, an alkyl is a C₁-C₄alkyl which represents a straight-chain or branched saturated monovalent hydrocarbon group having 1 to 4 carbon atoms. In some embodiments, an alkyl is a C₁-C₆ alkyl which represents a straight-chain or branched saturated monovalent hydrocarbon group having 1 to 6 carbon atoms. Examples of alkyl include without limitation methyl, ethyl, n-propyl, isopropyl, n-butyl, isobutyl, sec-butyl, and tert-butyl.

The term “alkylene” as used herein and unless otherwise indicated, refers to a divalent hydrocarbon chain that may be a straight chain or branched chain, containing the indicated number of carbon atoms or otherwise having from one to ten, one to eight, one to six or one to four carbon atoms. In certain embodiments, the hydrocarbon chain is optionally deuterated. Alkylene groups include but are not limited to methylene, ethylene, propylene and n-butylene. In certain embodiments, alkylene is methylene or ethylene.

The term “alkoxy” as used herein and unless otherwise indicated, refers to a group of formula —O-(alkyl). Alkoxy can be, for example, methoxy, ethoxy, n-propoxy, isopropoxy, n-butoxy, iso-butoxy, sec-butoxy, n-pentoxy, 2-pentoxy, 3-pentoxy, or hexyloxy. Likewise, the term “alkylthio” refers to a group of formula —S-(alkyl). The terms “haloalkoxy” and “haloalkylthio” refer to —O-(haloalkyl) and —S-(haloalkyl), respectively.

The term “amino” as used herein and unless otherwise indicated, refers to a group of the formula —NRR wherein each R is independently hydrogen, alkyl or haloalkyl.

The term “aryl” as used herein and unless otherwise indicated, is intended to mean any stable monocyclic or bicyclic carbon ring of up to 6 members in each ring, wherein at least one ring is aromatic. Examples of aryl include phenyl, naphthyl, tetrahydronaphthyl, indanyl, or biphenyl.

The term “aralkyl” as used herein and unless otherwise indicated, refers to a monovalent alkyl group in which an alkyl hydrogen atom is replaced by an aryl group. One of the carbons of the alkyl moiety serves as the point of attachment of the aralkyl group to another moiety. Non-limiting examples of “aralkyl” include benzyl, 2-phenylethyl, and 3-phenylpropyl groups.

The term “cycloalkyl” as used herein and unless otherwise indicated, refers to a monocyclic, bicyclic, tricyclic or other polycyclic hydrocarbon group having the indicated number of ring carbon atoms or otherwise having three to ten carbon atoms and which are fully saturated or partially unsaturated. Multicyclic cycloalkyl may be fused, bridged or spiro-ring systems. Cycloalkyl groups include, but are not limited to, cyclopropyl, cyclobutyl, cyclopentyl, cyclohexyl, cycloheptyl, cyclooctyl, norbornyl, and partially unsaturated hydrocarbon rings such as cyclobutylene, cyclopentene and cyclohexene. In some embodiments, cycloalkyl is a monocyclic C₃-C₈ cycloalkyl.

The term “cycloalkylalkyl” as used herein and unless otherwise indicated, refers to a monovalent alkyl group substituted with cycloalkyl.

The term “deuterium” as used herein and unless otherwise indicated, refers to the heavy isotope of hydrogen represented by the symbol D or ²H. As used herein, when a particular position in a compound is designated as “deuterated” or as having deuterium, it is understood that the compound is an isotopically enriched compound and the presence of deuterium at that position in the compound is substantially greater than its natural abundance of 0.0156%.

The term “enantiomerically pure” or “pure enantiomer” as used herein denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of a single enantiomer to the exclusion of its corresponding non-superimposable mirror image.

The term “halo”, “halogen” or “halide” as used herein and unless otherwise indicated, refers to a monovalent fluorine, chlorine, bromine or iodine group.

The term “haloalkyl” as used herein and unless otherwise indicated, refers to a monovalent alkyl group in which at least one hydrogen atom is replaced by a halogen. In some embodiments, more than one hydrogen atom (e.g., 2, 3, 4, 5 or 6) are replaced by halogens. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halogens (sometimes referred to herein as perhaloalkyl, e.g., perfluoroalkyl, such as trifluoromethyl).

The term “heterocycle”, “heterocyclyl” or “heterocyclic” as used herein and unless otherwise indicated, represents a stable 4-, 5-, 6- or 7-membered monocyclic- or a stable 6-, 7-, 8-, 9-, 10-, 11-, or 12-membered bicyclic heterocyclic ring system comprised of at least one non-aromatic (i.e. saturated or partially unsaturated) ring which consists of carbon atoms and from one to four, preferably up to three, heteroatoms selected from the group consisting of N, O and S, wherein the nitrogen and sulfur atoms may optionally be oxidized as N-oxide, sulfoxide or sulfone, and wherein the nitrogen atom may optionally be quaternized. A heterocycle can be bonded via a ring carbon atom or, if available, via a ring nitrogen atom. Bicyclic heterocyclic ring system(s) may be fused, bridged, or spiro-bicyclic heterocyclic ring system(s), and may be referred to as “bicyclic heterocyclyl” or “bicyclic heterocycle”. Examples of bicyclic heterocyclyl include, but are not limited to: bicyclic spirocycles, including but not limited to, 5-azaspiro[2.3]hexane, 2-azaspiro[3.3]heptane, 2-oxa-6-azaspiro[3.3]heptane, 2,6-diazaspiro[3.3]heptane, 2-thia-6-azaspiro[3.3]heptane, 1-oxa-6-azaspiro[3.3]heptane, 1,6-diazaspiro[3.3]heptane, 1-thia-6-azaspiro[3.3]heptane, 1-thia-6-azaspiro[3.3]heptane 1,1-dioxide, 7-oxa-2-azaspiro[3.5]nonane, 2, 7-diazaspiro[3.5]nonane, 7-thia-2-azaspiro [3.5]nonane, 2-oxa-6-azaspiro[3 .3]heptane, 1-oxa-7-azaspiro[4 .4]nonane, 1,7-diazaspiro[4.4]nonane, 1-thia-7-azaspiro[4.4]nonane, 1-thia-7-azaspiro[4.4]nonane, 6-oxa-2-azaspiro[3.4]octane, 2,6-diazaspiro[3.4]octane, 6-thia-2-azaspiro[3.4]octane, 2,6-diazaspiro[3.4]octan-7-one, 2,6-diazaspiro[3.4]octan-5-one, 5-oxa-2-azaspiro[3.4]octane, 2,5-diazaspiro[3.4] octane, 2,5-diazaspiro[3.4]octan-6-one, 5-thia-2-azaspiro[3.4]octane, 1-oxa-7-azaspiro[3.5]nonane, 1,7-diazaspiro[3.5]nonane, and 1-thia-7-azaspiro[3.5]nonane; bridged bicyclic heterocycles, including but not limited to, 6-oxa-3-azabicyclo[3.1.1]heptane, 3,6-diazabicyclo[3.1.1]heptane, 6-thia-3-azabicyclo[3.1.1]heptane, 3,6-diazabicyclo[3.1.1]heptane, 3-thia-6-azabicyclo[3.1.1]heptane, 2-oxa-5-azabicyclo[2.2.1]heptane, 2,5-diazabicyclo[2.2. 1]heptane, 2-thia-5-azabicyclo[2.2.1]heptane, 3,8-diazabicyclo[3.2.1]octane, 8-oxa-3-azabicyclo[3.2.1]octane, 8-thia-3-azabicyclo[3.2.1]octane, 2-oxa-5-azabicyclo[2.2.2]octane, 2,5-diazabicyclo[2.2.2]octane, 2-thia-5-azabicyclo[2.2.2]octane; and fused bicyclic heterocycles, including but not limited to, 3-azabicyclo[3.1.0]hexane, (1R,5S)-3-azabicyclo[3.1.0]hexane, hexahydro-1H-furo[3,4-c]pyrrole, (3aR,6aS)-hexahydro-1H-furo[3,4-c]pyrrole, octahydropyrrolo[3,4-c]pyrrole, (3aS,6aS)-octahydropyrrolo[3,4-c]pyrrole, hexahydro-1H-thieno[3,4-c]pyrrole and (3aR,6aS)-hexahydro-1H-thieno[3,4-c]pyrrole. In some embodiments, heterocyclyl is monocyclic having 4 to 7, preferably 4 to 6, ring atoms, of which 1 or 2 are heteroatoms independently selected from the group consisting of N, O and S. In some embodiments, heterocyclyl is monocyclic having 4 to 7, preferably 4 to 6, ring atoms, of which at least 1 heteroatom is N and the second heteroatom is N, O or S. In certain embodiments, the heterocyclyl is azetidine, pyrrolidine, piperidine, piperazine, morpholine or thiomorpholine. In some embodiments, a heterocyclyl group is bicyclic, and in which case, the second ring may be an aromatic or a non-aromatic ring which consists of carbon atoms and from one to four, preferably up to three, heteroatoms independently selected from the group consisting of N, O and S, or the second ring may be a benzene ring, or a “cycloalkyl”, or a “cycloalkenyl”, as defined herein. Other examples of heterocyclic groups include, but are not limited to azetidine, chroman, dihydrofuran, dihydropyran, dioxane, dioxolane, hexahydroazepine, imidazolidine, imidazoline, indoline, isochroman, isoindoline, isothiazoline, isothiazolidine, isoxazoline, isoxazolidine, morpholine, oxazoline, oxazolidine, oxetane, piperazine, piperidine, dihydropyridine, tetrahydropyridine, dihydropyridazine, pyran, pyrazolidine, pyrazoline, pyrrolidine, pyrroline, tetrahydrofuran, tetrahydropyran, thiamorpholine, tetrahydrothiophene, thiazoline, thiazolidine, thiomorpholine, thietane, thiolane, sulfolane, 1,3-dioxolane, 1,3-oxazolidine, 1,3-thiazolidine, tetrahydrothiopyran, tetrahydrotriazine, 1,3-dioxane, 1,4-dioxane, hexahydrotriazine, tetrahydro-oxazine, tetrahydropyrimidine, perhydroazepine, perhydro-1,4-diazepine, perhydro-1,4-oxazepine, 7-azabicyclo[2.2.1]heptane, 3-azabicyclo[3.2.0]heptane, 7-azabicyclo[4.1.0]heptane, 2,5-diazabicyclo[2.2.1]heptane, 2-oxa-5-azabicyclo[2.2.1]heptane, tropane, 2-oxa-6-azaspiro [3.3]heptane, dihydrobenzofuran, diydrobenzimidazolyl, dihydrobenzoxazole, and dihydrobenzothiazolyl, and N-oxides or sulfones or sulfoxides thereof.

The term “heterocyclylalkyl” as used herein and unless otherwise indicated, refers to a monovalent alkyl group substituted with heterocyclyl.

The term “heteroaryl”, as used herein and unless otherwise indicated, represents a stable 5-, 6- or 7-membered monocyclic- or stable 9- or 10-membered fused bicyclic ring system which comprises at least one aromatic ring, which consists of carbon atoms and from one to four, preferably up to three, heteroatoms selected from the group consisting of N, O and S wherein the nitrogen and sulfur heteroatoms or a carbon atom of the heteroaryl may optionally be oxidized, and the nitrogen heteroatom may optionally be quaternized. In the case of a “heteroaryl” which is a bicyclic group, the second ring need not be aromatic and need not comprise a heteroatom. Accordingly, bicyclic “heteroaryl” includes, for example, a stable 5- or 6-membered monocyclic aromatic ring consisting of carbon atoms and from one to four, preferably up to three, heteroatoms, as defined immediately above, fused to a benzene ring, or a second monocyclic “heteroaryl”, or a “heterocyclyl”, a “cycloalkyl”, or a “cycloalkenyl”, as defined above. Examples of heteroaryl groups include, but are not limited to, benzimidazole, benzopyrazole, benzisothiazole, benzisoxazole, benzofuran, isobenzofuran, benzothiazole, benzothiophene, benzotriazole, benzoxazole, furan, furazan, imidazole, indazole, indole, indolizine, isoquinoline, isothiazole, isoxazole, naphthyridine, oxadiazole, oxazole, phthalazine, pteridine, purine, pyrazine, pyrazole, pyridazine, pyridine, pyrimidine, pyrrole, pyridinone, quinazoline, quinoline, quinoxaline, tetrazole, thiadiazole, thiazole, thiophene, triazine, triazole, benzimidazole, benzothiadiazole, isoindole, pyrrolopyridines, imidazopyridines such as imidazo[1,2-a]pyridine, pyrazolopyridine, pyrrolopyrimidine and N-oxides thereof.

The term “heteroaralkyl” or “heteroarylalkyl” as used herein refers to a monovalent alkyl group substituted with heteroaryl.

The term “hydrate” as used herein and unless otherwise indicated, refers to a compound provided herein or a salt thereof, that further includes a stoichiometric or non-stoichiometric amount of water bound by non-covalent intermolecular forces.

The term “solvate” as used herein and unless otherwise indicated, refers to a solvate formed from the association of one or more solvent molecules to a compound provided herein. The term “solvate” includes hydrates (e.g., mono-hydrate, dehydrate, trihydrate, and the like).

The term “subject” as used herein and unless otherwise indicated, refers to any human or veterinary subject, including mammals such as mice, rats, cows, sheep, pigs, rabbits, goats, horses, monkeys, dogs, cats, and humans, including neonatal, infant, juvenile, adolescent, adult or geriatric patients.

The term “thiol” refers to a group having the formula —SH.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The term “treating”, “treat”, or “treatment” refers generally to controlling, alleviating, ameliorating, slowing the progress of, delaying the onset, reducing the risk of developing, or eliminating a disease, disorder or condition, or a symptom of a disease, disorder or condition. In addition to its customary meaning, the term “preventing”, “prevent”, or “prevention” also refers to delaying the onset of, or reducing the risk of developing a disease, disorder or condition or of a process that can lead to the disease, disorder or condition, or the recurrence of symptoms of a disease, disorder or condition.

Unless stated otherwise or specifically described, it is understood that substitutions where present can occur on any atom of the alkyl, alkenyl, alkynyl, cycloalkyl, heterocyclyl, aryl or heteroaryl groups.

Unless specifically stated otherwise, where a compound may assume alternative tautomeric or stereoisomeric forms, all alternative isomers are intended to be encompassed within the scope of the claimed subject matter. For example, where a compound is described as having one of two tautomeric forms, it is intended that both tautomers be encompassed herein. Thus, the compounds provided herein may be enantiomerically pure, or be enantiomeric mixtures or diastereomeric mixtures.

It is to be understood that the compound provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

In the description herein, if there is any discrepancy between a chemical name and chemical structure, the chemical structure controls.

### Compounds

In certain embodiments, provided herein are compounds of Formula (I):

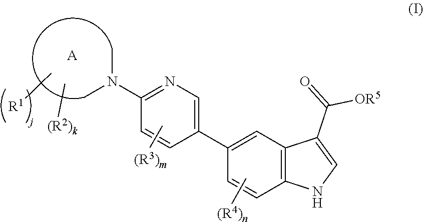

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl —R^{u}C(O)OR⁹, —R^{u}C(O)NR⁷R⁸, —R^{u}C(O)R¹¹, —R^{u}NR¹⁰ C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
each R⁴ is independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

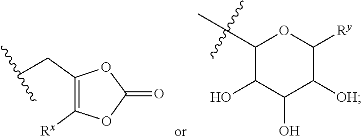

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
R⁷ and R⁸ are each independently hydrogen, alkyl, haloalkyl or alkoxyalkyl; or
R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3;
m is 1, 2 or 3;
n is 1 or 2; and
each t is independently 0, 1 or 2.

In certain embodiments, provided herein are compounds having the Formula (Ia):

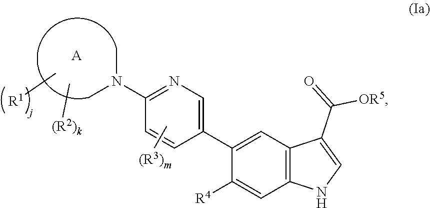

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
each R⁴ is independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

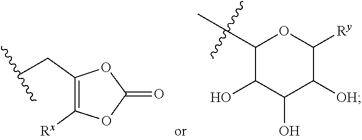

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
   (i) R⁷ and R⁸ are each independently hydrogen, alkyl, haloalkyl or alkoxyalkyl; or
   (ii) R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3;
m is 1, 2 or 3;
n is 1 or 2; and
each t is independently 0, 1 or 2.

In certain embodiments, provided herein are compounds having the Formula (Ib):

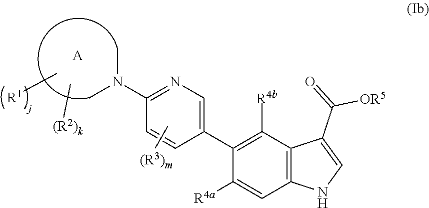

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C (O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
R^{4a} and R^{4b} are each independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

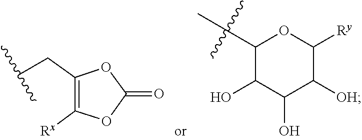

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
   (i) R⁷ and R⁸ are each independently hydrogen, alkyl, haloalkyl or alkoxyalkyl; or
   (ii) R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3;
m is 1, 2 or 3;
n is 1 or 2; and
each t is independently 0, 1 or 2.

In certain embodiments, provided herein are compounds having the Formula (Ic):

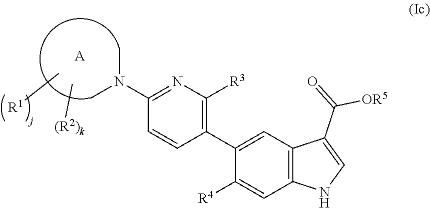

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸ , —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
each R⁴ is independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

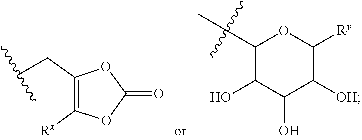

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
   (i) R⁷ and R⁸ are each independently hydrogen, alkyl, haloalkyl or alkoxyalkyl; or
   (ii) R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3; and
each t is independently 0, 1 or 2.

In yet certain embodiments, provided herein are compounds having the Formula (Id):

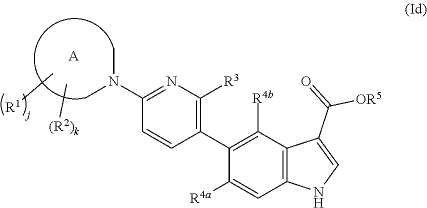

or a pharmaceutically acceptable salt, solvate, hydrate, a single stereoisomer, a mixture of stereoisomers or an isotopic variant thereof, wherein:
Ring A is a bicyclic heterocyclyl;
each R¹ is independently alkyl, haloalkyl, cyanoalkyl, cycloalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
R^{4a} and R^{4b} are each independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v},

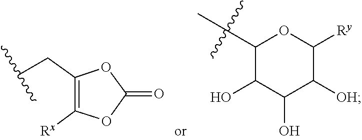

each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
   (i) R⁷ and R⁸ are each independently hydrogen, alkyl, haloalkyl or alkoxyalkyl; or
   (ii) R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3; and
each t is independently 0, 1 or 2.

In certain embodiments, provided herein are compounds of Formula (I), (Ia), (Ib), (Ic) or (Id) wherein the bicyclic heterocyclyl of Ring A is a spirocycle. In certain embodiments, provided herein are compounds of Formula (I), (Ia), (Ib), (Ic) or (Id) wherein Ring A is

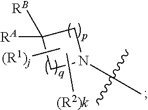

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; and wherein j, k, R¹ and R² are as described for Formula (I), (Ia), (Ib), (Ic) or (Id), respectively, and R¹ and R² may be on either ring of the spirocycle.

In certain embodiments, provided herein are compounds of Formula (I), (Ia), (Ib), (Ic) or (Id) wherein Ring A is

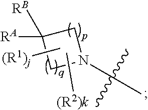

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; each R¹ is independently alkyl, haloalkyl, cyanoalkyl, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}NR⁷R⁸, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is

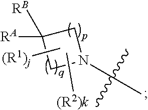

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is

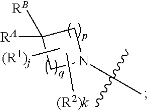

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl and j and k are as described for Formula (I), (Ia), (Ib), (Ic) or (Id), respectively.

In certain embodiments, provided herein are compounds of Formula (I), (Ia), (Ib), (Ic) or (Id) wherein Ring A is

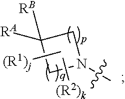

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and j and k are as described for Formula (I), (Ia), (Ib), (Ic) or (Id), respectively.

In certain embodiments, provided herein are compounds of Formula (I), (Ia), (Ib), (Ic) or (Id) wherein Ring A is

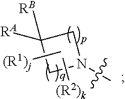

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; R^{l} is alkyl, haloalkyl, cyanoalkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; R² is halo, oxo, cyano, alkyl, cyanoalkyl, haloalkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; j and k are each independently 0 or 1; and R^{u}, R⁶, R⁷, R⁸, R⁹ and R¹⁰ are as described for Formula (I), (Ia), (Ib), (Ic) or (Id), respectively.

In certain embodiments, provided herein are compounds of Formula (I), (Ia), (Ib), (Ic) or (Id) wherein Ring A is

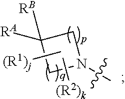

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; R¹ is C₁₋₃alkyl, haloC₁₋₃alkyl, cyanoC₁₋₃alkyl, —R^{u}NR⁷R⁸, —R^{u}C(O)OR⁹, —R^{u}C(O)NR⁷R⁸ or —R^{u}NR¹⁰C(O)OR⁹; R² is halo, oxo, cyano, C₁₋₃alkyl, haloC₁₋₃alkyl, cyanoC₁₋₃alkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; each R^{u} is independently a direct bond, methylene or ethylene; each R⁶, R⁷, R⁸ and R¹⁰ are each independently hydrogen, C₁₋₃alkyl or haloC₁₋₃alkyl; each R⁹ is independently hydrogen, C₁₋₄alkyl or haloC₁₋₄alkyl; and j and k are each independently 0 or 1.

In certain embodiments, provided herein are compounds of Formula (I), (Ia), (Ib), (Ic) or (Id) wherein Ring A is

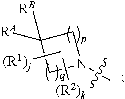

R^{A} and R^{B}, together with the carbon atom to which they are attached, form cycloalkyl or heterocyclyl; p is 0, 1, 2 or 3; q is 0, 1, 2 or 3, provided the sum of p and q are 2, 3, 4 or 5; R¹ is C₁₋₃ alkyl, haloC₁₋₃alkyl, hydroxyC₁₋₃alkyl, hydroxyl, —R^{u}NR⁷R⁸ or —R^{u}C(O)OR⁹; R² is oxo, C₁₋₃alkyl, haloC₁₋₃alkyl, hydroxyC₁₋₃alkyl, hydroxyl, —R^{u}NR⁷R⁸, —R^{u}C(O)OR⁹ or —R^{u}NR¹⁰C(O)OR⁹; each R^{u} is independently a direct bond, methylene or ethylene; R⁷ and R⁸ are each independently hydrogen or C₁₋₃alkyl; R⁹ is C₁₋₄alkyl; and j and k are each independently 0 or 1.

In certain embodiments, provided herein are compounds of Formula (I), (Ia), (Ib), (Ic) or (Id) wherein Ring A is:

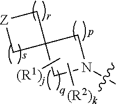

wherein Z is —N(R^{C})—, —N(R^{G})S(O)ₜ—, —O—, —S(O)ₜ—, or —C(R^{E})(R^{F})—; R^{C} is hydrogen or R¹;
R^{E} and R^{F} are each independently hydrogen or R²; R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5; t is 0, 1 or 2; and
wherein j, k, R¹ and R² are as described for Formula (I), (Ia), (Ib), (Ic) or (Id) and R¹ and R² may be on either ring of the spirocycle. In certain embodiments, r, s, p and q are all 1. In certain embodiments, Z is —O— and r, s, p and q are all 1.

In certain embodiments, provided herein are compounds of Formula (I), (Ia), (Ib), (Ic) or (Id) wherein Ring A is:

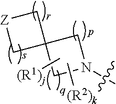

wherein Z is —N(R^{C})—, —N(R^{G})S(O)ₜ—, —O—, —S(O)ₜ—, or —C(R^{E})(R^{F})—; R^{C} is hydrogen or R¹;
R^{E} and R^{F} are each independently hydrogen or R²; R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2; each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C (O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively. In certain embodiments, r, s, p and q are all 1. In certain embodiments, Z is —O— and r, s, p and q are all 1.

In certain embodiments, provided herein are compounds of Formula (I), (Ia), (Ib), (Ic) or (Id) wherein Ring A is:

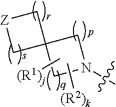

wherein Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2; each each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively. In certain embodiments, r, s, p and q are all 1. In certain embodiments, Z is —O— and r, s, p and q are all 1.

In certain embodiments, provided herein are compounds of Formula (I), (Ia), (Ib), (Ic) or (Id) wherein Ring A is:

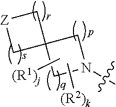

wherein Z is —N(R^{C})—, —N(R^{G})S(O)ₜ—, —O—, —S(O)ₜ—, or —C(R^{E})(R^{F})—; R^{C} is hydrogen or R¹;
R^{E} and R^{F} are each independently hydrogen or R²; R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2; each R¹ is independently C₁₋₃ alkyl, haloC₁₋₃ alkyl, cyanoC₁₋₃alkyl, —R^{u}OR⁶, —R^{u}NR⁷R⁸, —R^{u}C(O)NR⁷R⁸, —R^{u}C(O)R¹¹, —R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl and j and k are as described for Formula (I), (Ia), (Ib), (Ic) or (Id), respectively. In certain embodiments, r, s, p and q are all 1. In certain embodiments, Z is —O— and r, s, p and q are all 1.

In certain embodiments, provided herein are compounds of Formula (I), (Ia), (Ib), (Ic) or (Id) wherein Ring A is:

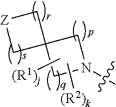

wherein Z is —N(R^{C})—, —N(R^{G})S(O)ₜ—, —O—, —S(O)ₜ—, or —C(R^{E})(R^{F})—; R^{C} is hydrogen or R¹;
R^{E} and R^{F} are each independently hydrogen or R²; R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5; t is 0, 1 or 2;
each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and j and k are as described for Formula (I), (Ia), (Ib), (Ic) or (Id), respectively. In certain embodiments, r, s, p and q are all 1. In certain embodiments, Z is —O— and r, s, p and q are all 1.

In certain embodiments, provided herein are compounds of Formula (I), (Ia), (Ib), (Ic) or (Id) wherein Ring A is selected from:

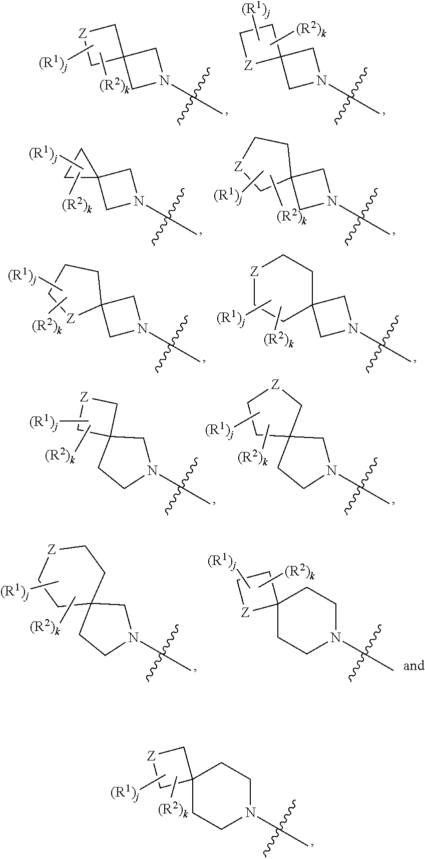

wherein Z, R¹, R², j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

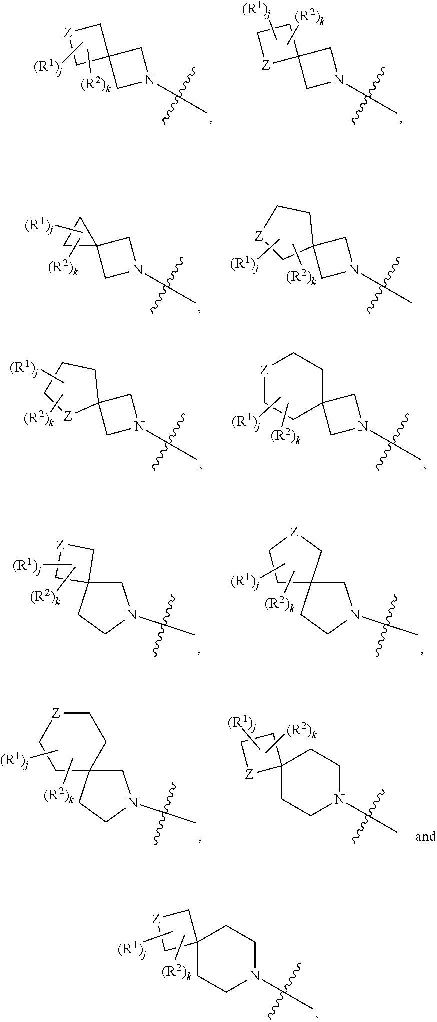

wherein each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; and Z, R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

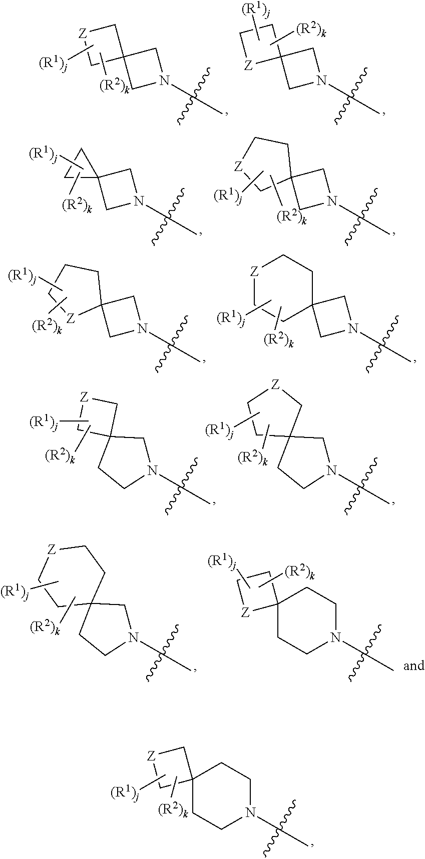

wherein each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and Z, R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

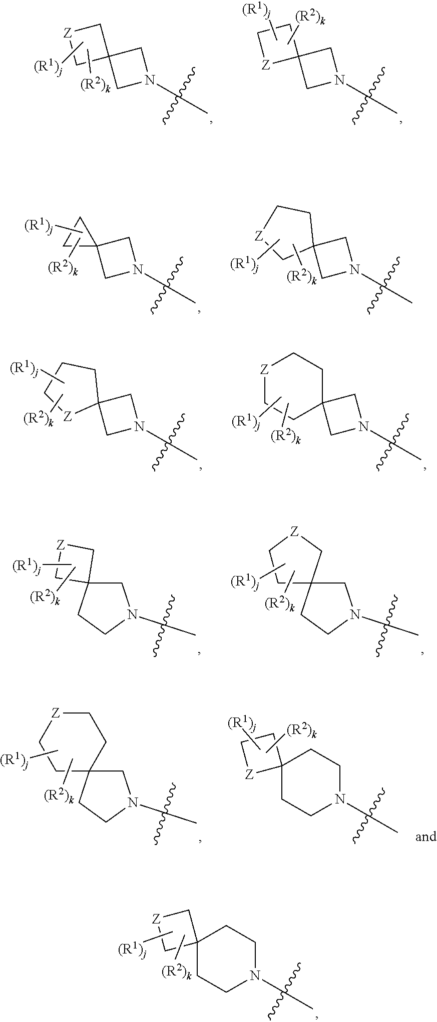

wherein each R¹ is independently C₁₋₃ alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl and Z, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

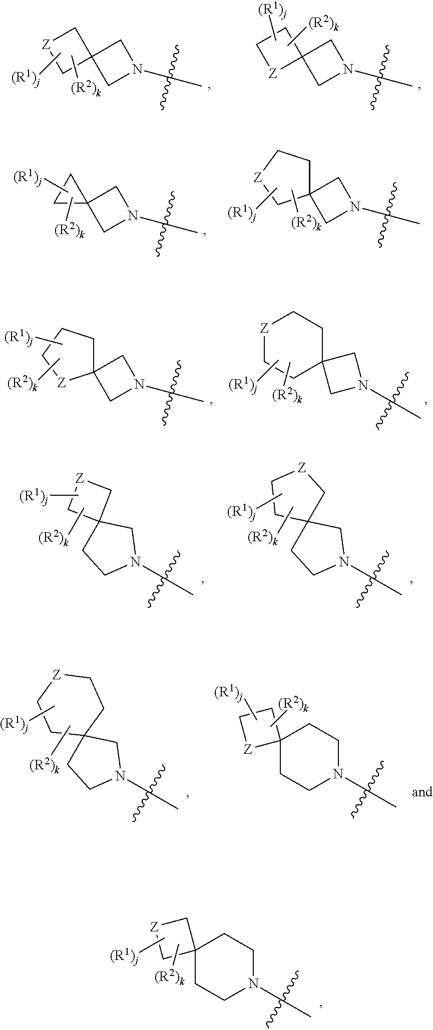

wherein each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and Z, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

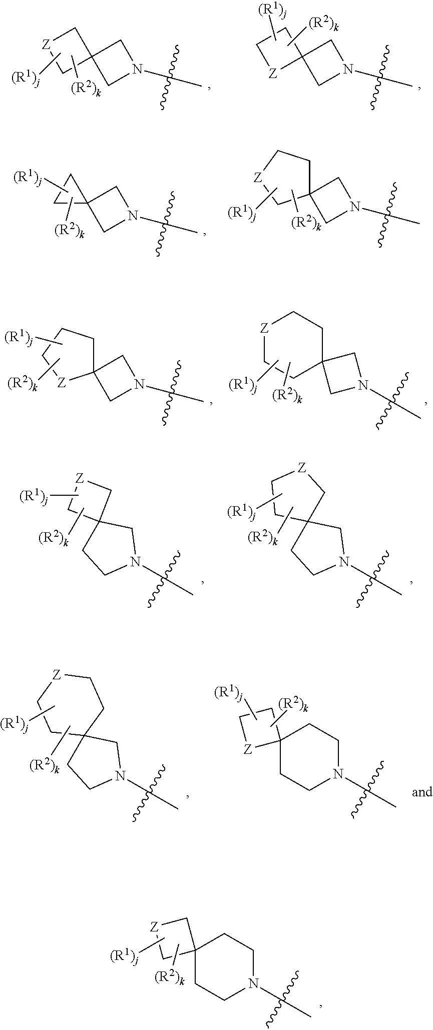

wherein R¹ is alkyl, haloalkyl, cyanoalkyl, —R^{u}OR⁷R⁸, —R^{u}C(O)OR⁹, —R^{u}C(O)NR⁷R⁸ or —R^{u}NR¹⁰C(O)OR⁹; R² is halo, oxo, cyano, alkyl, cyanoalkyl, haloalkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; j and k are each independently 0 or 1; and Z, R⁶, R⁷, R⁸, R⁹ and R¹⁰ are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

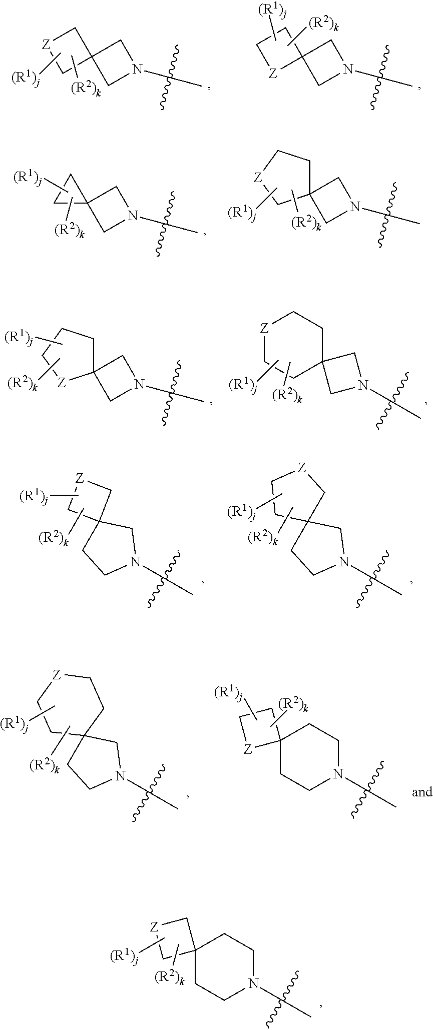

wherein R¹ is C₁₋₃alkyl, haloC₁₋₃alkyl, cyanoC₁₋₃alkyl, —R^{u}OR⁶, —R^{u}C(O)OR⁹, —R^{u}C(O)NR⁷R⁸ or —R^{u}NR¹⁰C(O)OR⁹; R² is halo, oxo, cyano, C₁₋₃alkyl, haloC₁₋₃alkyl, cyanoC₁₋₃alkyl, —R^{u}C(O)OR⁹, —R^{u}C(O)NR⁷R⁸ or —R^{u}NR¹⁰C(O)OR⁹; each R^{u} is independently a direct bond, methylene or ethylene; each R⁶, R⁷, R⁸ and R¹⁰ are each independently hydrogen, C₁₋₃alkyl or haloC₁₋₃alkyl; each R⁹ is independently hydrogen, C₁₋₄alkyl or haloC₁₋₄alkyl; j and k are each independently 0 or 1 and Z is as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

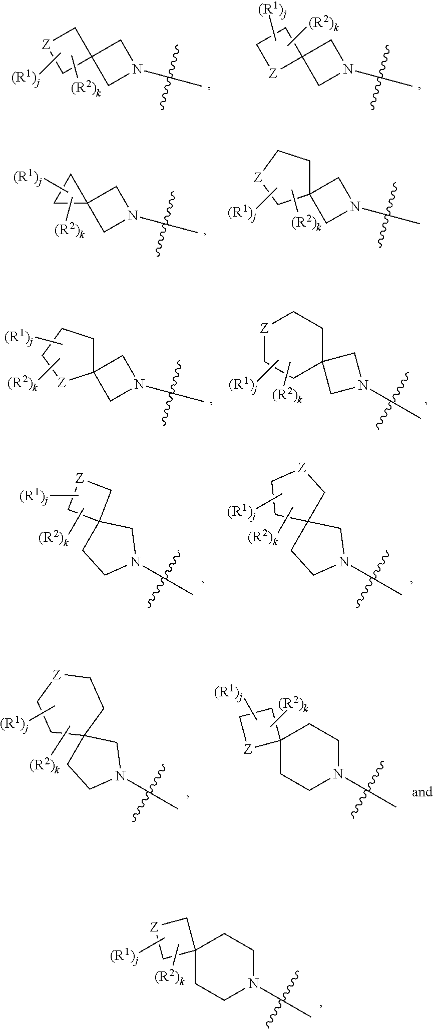

wherein each R¹ is C₁₋₃alkyl, haloC₁₋₃alkyl, hydroxyC₁₋₃alkyl, hydroxyl, —R^{u}NR⁷R⁸ or —R^{u}C(O)OR⁹; R¹ is oxo, C₁₋₃alkyl, haloC₁₋₃alkyl, hydroxyC₁₋₃alkyl, hydroxyl, —R^{u}NR⁷R⁸, —R^{u}C(O)OR⁹ or —R^{u}NR¹⁰C(O)OR⁹; each R^{u} is independently a direct bond, methylene or ethylene; R⁷ and R⁸ are each independently hydrogen or C₁₋₃alkyl; R⁹ is C₁₋₄alkyl; and j and k are each independently 0 or 1 and Z is as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

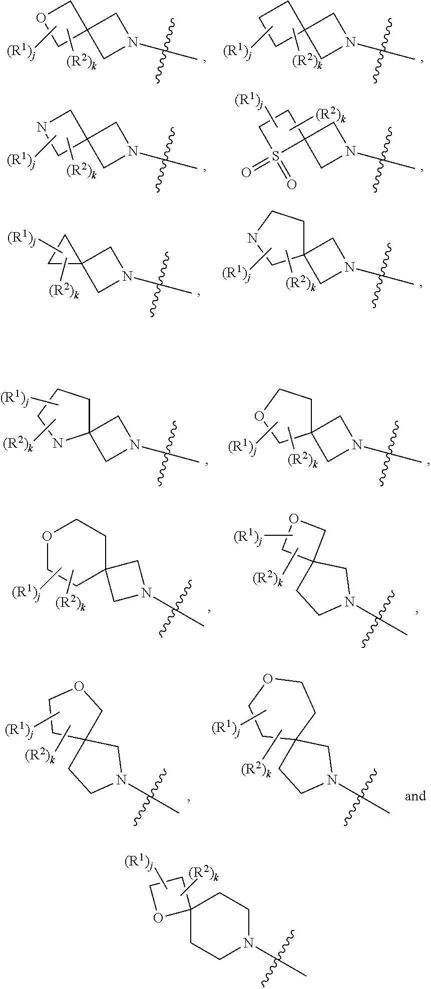

wherein R¹, R², j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

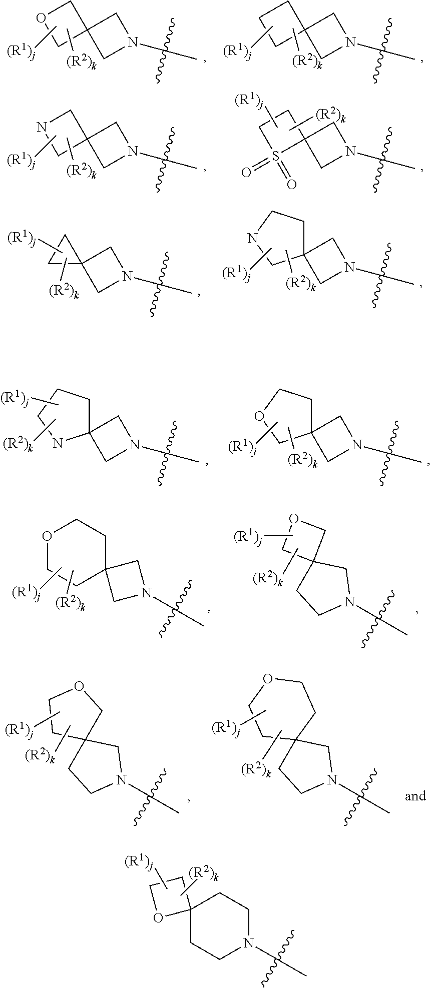

wherein each R¹ is independently alkyl, haloalkyl, cyanoalkyl, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

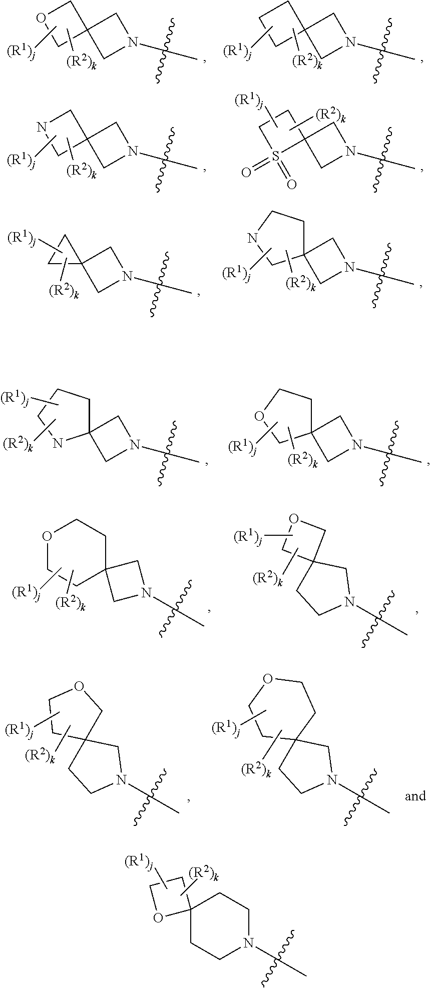

wherein each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

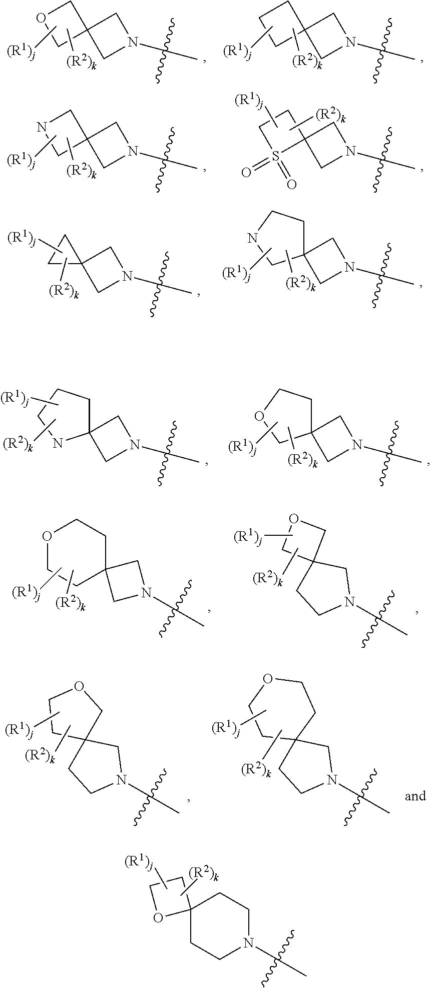

wherein each R¹ is independently C₁₋₃alkyl haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl and j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

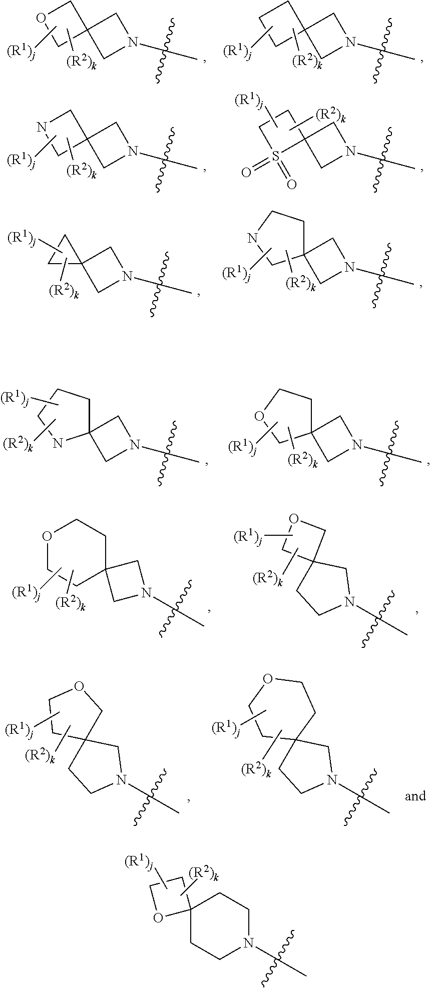

wherein each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

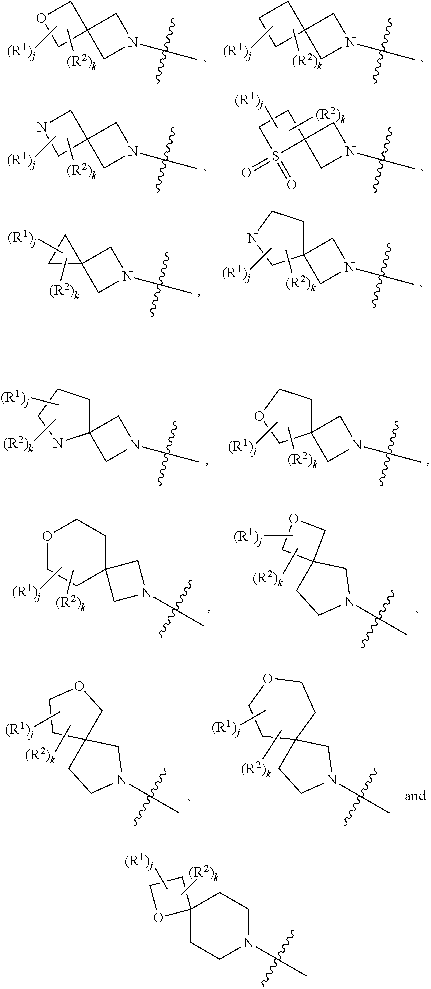

wherein R¹ is alkyl, haloalkyl, cyanoalkyl, —R^{u}C(O)OR⁹, —R^{u}C(O)NR⁷R⁸ or —R^{u}NR¹⁰C(O)OR⁹; R² is halo, oxo, cyano, alkyl, cyanoalkyl, haloalkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; j and k are each independently 0 or 1;
and R^{u}, R⁶, R⁷, R⁸, R⁹ and R¹⁰ are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

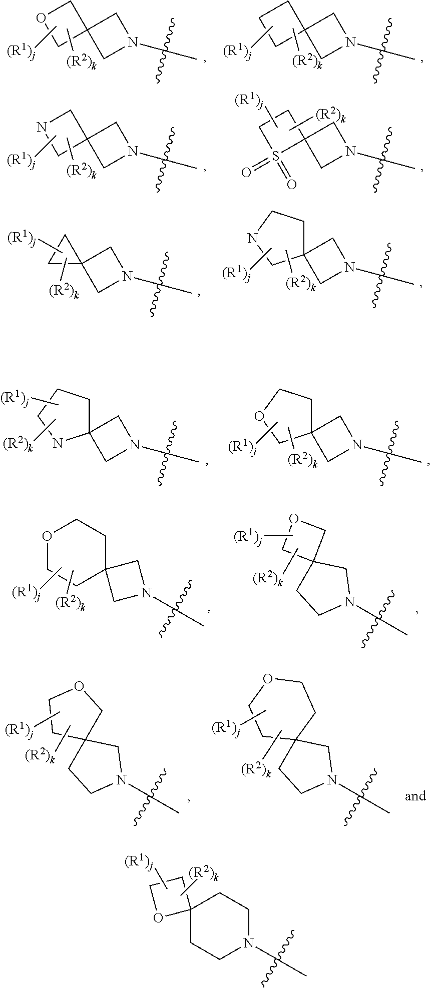

wherein R¹ is C₁₋₃alkyl, haloC₁₋₃alkyl, cyanoC₁₋₃alkyl, —R^{u}OR⁶, —R^{u}NR⁷R⁸, —R^{u}C(O)OR⁹, —R^{u}C(O)NR⁷R⁸ or —R^{u}NR¹⁰C(O)OR⁹; R² is halo, oxo, cyano, C₁₋₃alkyl, haloC₁₋₃ alkyl, cyanoC₁₋₃alkyl, —R^{u}OR⁶, —R^{u}C(O)OR⁹, —R^{u}C(O)NR⁷R⁸ or —R^{u}NR¹⁰C(O)OR⁹; each R^{u} is independently a direct bond, methylene or ethylene; each R⁶, R⁷, R⁸ and R¹⁰ are each independently hydrogen, C₁₋₃alkyl or haloC₁₋₃alkyl; each R⁹ is independently hydrogen, C₁₋₄alkyl or haloC₁₋₄alkyl; and j and k are each independently 0 or 1.

In certain embodiments, provided herein are compounds of Formula (I), (Ia), (Ib), (Ic) or (Id) wherein Ring A is selected from:

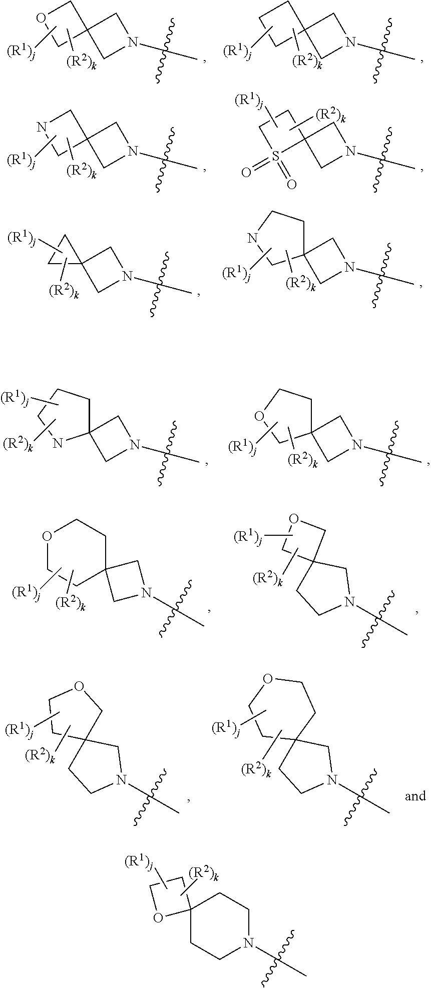

wherein R¹ is C₁₋₃alkyl, haloC₁₋₃alkyl, hydroxyC₁₋₃alkyl, hydroxyl, —R^{u}NR⁷R⁸ or —R^{u}C(O)OR⁹; R² is oxo, C₁₋₃alkyl, haloC₁₋₃alkyl, hydroxyC₁₋₃alkyl, hydroxyl, —R^{u}NR⁷R⁸, —R^{u}C(O)OR⁹ or —R^{u}NR¹⁰(O)OR⁹; each R^{u} is independently a direct bond, methylene or ethylene; R⁷ and R⁸ are each independently hydrogen or C₁₋₃alkyl; R⁹ is C₁₋₄alkyl; and j and k are each independently 0 or 1.

In certain embodiments, provided herein are compounds of Formula (I), (Ia), (Ib), (Ic) or (Id), wherein Ring A

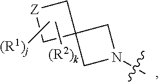

is wherein Z, j, k, R¹ and R² are as described for Formula (I), (Ia), (Ib), (Ic) or (Id), respectively. In certain embodiments, provided herein are compounds of Formula (I), (Ia), (Ib), (Ic) or (Id), wherein Ring A is

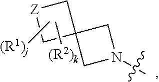

j and k are each independently 0 or 1; wherein Z, R¹ and R² are as described for Formula (I), (Ia), (Ib), (Ic) or (Id), respectively. In certain embodiments, j and k are both 0. In certain embodiments, Z is —O—, —S—, —S(O)— or —S(O)₂— or —C(H)(R²)—; j is 0 and R² is as described for Formula (I), (Ia), (Ib), (Ic) or (Id), respectively. In certain embodiments, Z is —O—, —S—, —S(O)— or —S(O)₂— or —CH₂—. In certain embodiments, Z is —O—, —S—, —S(O)— or —S(O)₂—. In yet certain embodiments, Z is —O— or —S—. In yet certain embodiments, Z is —O— or —CH₂—.

In certain embodiments, provided herein are compounds of Formula (I), (Ia), (Ib), (Ic) or (Id), wherein Ring A is

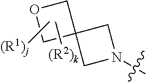

wherein j, k, R¹ and R² are as described for Formula (I), (Ia), (Ib), (Ic) or (Id), respectively. In certain embodiments, provided herein are compounds of Formula (I), (Ia), (Ib), (Ic) or (Id), wherein Ring A is

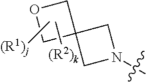

wherein j and k are each independently 0 or 1; and R¹ and R² are as described for Formula (I), (Ia), (Ib), (Ic) or (Id), respectively. In certain embodiments, j and k are both 0.

In certain embodiments, provided herein are compounds of Formula (I), (Ia), (Ib), (Ic) or (Id) wherein the bicyclic heterocyclyl of Ring A is a fused bicyclic heterocyclyl.

In certain embodiments, provided herein are compounds of Formula (I), (Ia), (Ib), (Ic) or (Id) wherein Ring A is

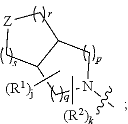

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and R¹, R², j and k are as described for claim 1 and R¹ and R² may be on either ring of the fused bicyclic heterocyclyl. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

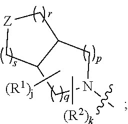

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; R¹ and R² may be on either ring of the fused bicyclic heterocyclyl and R^{u}, R⁶, R⁷, R⁸, R⁹, R¹⁰, R¹¹, j and k are as described for Formula (I), (Ia), (Ib), (Ic) or (Id), respectively. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

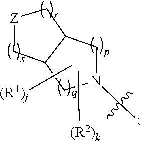

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; R¹ and R² may be on either ring of the fused bicyclic heterocyclyl and R⁶, R⁷, R⁸, R⁹, R¹⁰, R¹¹, j and k are as described for Formula (I), (Ia), (Ib), (Ic) or (Id), respectively. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

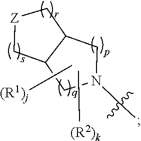

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁-alkyl, haloC₁₋₄alkyl or cyanoC₁₋₄alkyl; each R¹¹ is independently C₁₋₃alkyl or haloC₁₋₃alkyl; R¹ and R² may be on either ring of the fused bicyclic heterocyclyl and j and k are as described for Formula (I), (Ia), (Ib), (Ic) or (Id), respectively. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

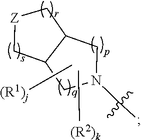

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; R¹ and R² may be on either ring of the fused bicyclic heterocyclyl; each R^{u} is independently a direct bond, methylene or ethylene; and each R⁶ is hydrogen, C₁₋₃alkyl or haloC₁₋₃alkyl; and j and k are as described for Formula (I), (Ia), (Ib), (Ic) or (Id), respectively. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

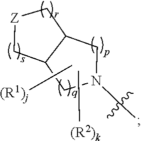

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; R¹ is alkyl, haloalkyl, cyanoalkyl, —R^{u}OR⁶ or —R^{u}C(O)R¹¹; R² is halo, cyano, alkyl, haloalkyl, cyanoalkyl, —R^{u}OR⁶ or —R^{u}C(O)R¹¹; R¹ and R² may be on either ring of the fused bicyclic heterocyclyl; j and k are each independently 0 or 1; and R^{u}, R⁶ and R¹¹ are as described for Formula (I), (Ia), (Ib), (Ic) or (Id), respectively. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

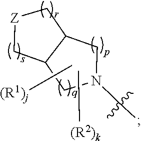

Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; R¹ is C₁₋₃alkyl, haloC₁₋₃alkyl, cyanoC₁₋₃alkyl, —R^{u}OR⁶ or —R^{u}C(O)R¹¹; R² is halo, cyano, C₁₋₃alkyl, haloC₁₋₃alkyl, cyanoC₁₋₃alkyl, —R^{u}OR⁶ or —R^{u}C(O)R¹¹; R¹ and R² may be on either ring of the fused bicyclic heterocyclyl; is a direct bond, methylene or ethylene; R⁶ is hydrogen or C₁₋₃alkyl; R¹¹ is C₁₋₃alkyl; and j and k are each independently 0 or 1. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

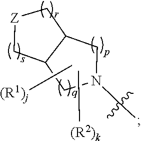

Z is —N(R^{C})—, —N(R^{G})S(O)ₜ—, —O—, —S(O)ₜ—, or —C(R^{E})(R^{F})—,
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; R¹ is C₁₋₃alkyl, haloC₁₋₃alkyl, —R^{u}OR⁶ or —R^{u}C(O)R¹¹; R² is cyano, C₁₋₃alkyl or —R^{u}OR⁶; R¹ and R² may be on either ring of the fused bicyclic heterocyclyl; R^{u} is a direct bond, methylene or ethylene; R⁶ is hydrogen or C₁₋₃alkyl; R¹¹ is C₁₋₃alkyl; and j and k are each independently 0 or 1. In certain embodiments, r, s, p and q are all 1. In certain embodiments, Z is —O— and r, s, p and q are all 1. In certain embodiments, Z is —O— and r, s, p and q are all 1, and j and k are both 0.

In certain embodiments, provided herein are compounds of Formula (I), (Ia), (Ib), (Ic) or (Id) wherein Ring A is

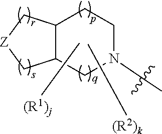

p is 0, 1 or 2 and wherein Z, R¹, R², j, k, q, r and s are as described for Formula (I), (Ia), (Ib), (Ic) or (Id), respectively. In certain embodiments, p is 0 or 1 and q is 0 or 1 provided p and q are not both 0.

In certain embodiments, provided herein are compounds of Formula (I), (Ia), (Ib), (Ic) or (Id) wherein Ring A is selected from:

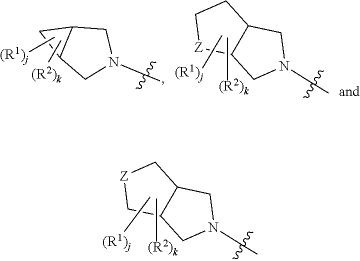

wherein Z, R¹, R², j and k, are as described for Formula (I), (Ia), (Ib), (Ic) or (Id), respectively. In certain embodiments, j and k are both 0.

In certain embodiments, provided herein are compounds of Formula (I), (Ia), (Ib), (Ic) or (Id) wherein Ring A is selected from:

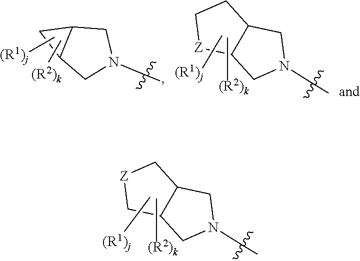

wherein each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹ or —R^{u}NR¹⁰C(O)OR⁹; and Z, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

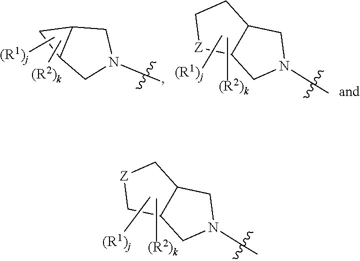

wherein each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and Z, R^{u}, R⁶, R⁷, R⁸, R⁹, R¹⁰, R¹¹, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

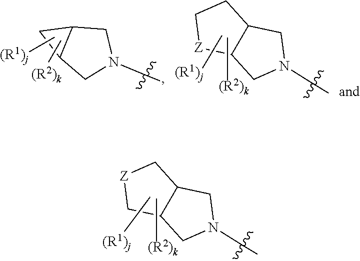

wherein each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl and Z, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

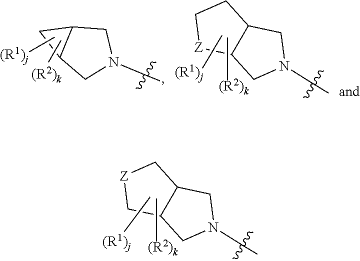

wherein R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and Z, j and k are as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

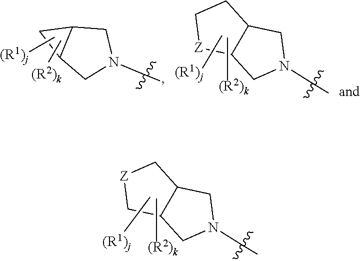

wherein R¹ is alkyl, haloalkyl, cyanoalkyl, —R^{u}OR⁶ or —R^{u}C(O)R¹¹; R² is halo, cyano, alkyl, haloalkyl, cyanoalkyl, —R^{u}OR⁶ or —R^{u}C(O)R¹¹; j and k are each independently 0 or 1; and R^{u}, R⁶ and R¹¹ are as described; and Z is as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

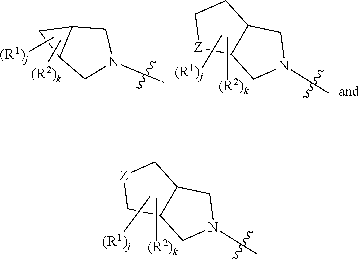

wherein R¹ is C₁₋₃alkyl, haloC₁₋₃alkyl, cyanoC₁₋₃alkyl, —R^{u}OR⁶ or —R^{u}C(O)R¹¹; R² is halo, cyano, C₁₋₃alkyl, haloC₁₋₃alkyl, cyanoC₁₋₃alkyl, —R^{u}OR⁶ or —R^{u}C(O)R¹¹; R^{u} is a direct bond, methylene or ethylene; R⁶ is hydrogen or C₁₋₃alkyl; R¹¹ is C₁₋₃alkyl; and j and k are each independently 0 or 1; and Z is as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

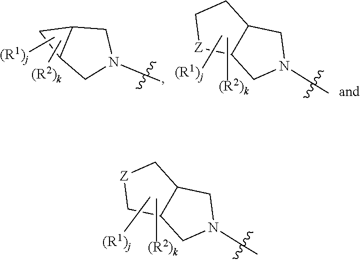

wherein R¹ is C₁₋₃alkyl, haloC₁₋₃alkyl, —R^{u}OR⁶ or —R^{u}C(O)R¹¹; R² is cyano, C₁₋₃alkyl or —R^{u}OR⁶; R^{u} is a direct bond, methylene or ethylene; R⁶ is hydrogen or C₁₋₃alkyl; R¹¹ is C₁₋₃alkyl; and j and k are each independently 0 or 1; and Z is as described for Formula (I), (Ia), (Ib), (Ic) or (Id), respectively.

In certain embodiments, provided herein are compounds of Formula (I), (Ia), (Ib), (Ic) or (Id) wherein Ring A is selected from:

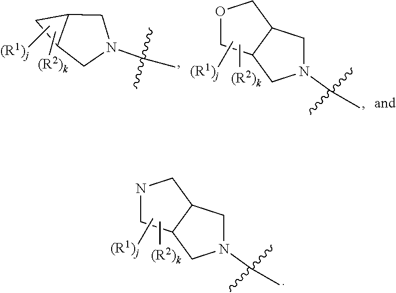

wherein R¹, R², j and k, are as described for Formula (I), (Ia), (Ib), (Ic) or (Id), respectively. In certain embodiments, j and k are both 0.

In certain embodiments, provided herein are compounds of Formula (I), (Ia), (Ib), (Ic) or (Id) wherein Ring A is

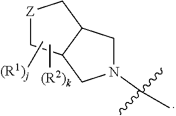

wherein Z, R¹, R², j and k, are as described for Formula (I), (Ia), (Ib), (Ic) or (Id), respectively. In certain embodiments, j and k are both 0.

In certain embodiments, provided herein are compounds of Formula (I), (Ia), (Ib), (Ic) or (Id) wherein Ring A is

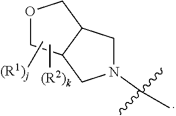

wherein R¹, R², j and k are as described for Formula (I), (Ia), (Ib), (Ic) or (Id), respectively. In certain embodiments, j and k are both 0.

In certain embodiments, provided herein are compounds of Formula (I), (Ia), (Ib), (Ic) or (Id) wherein Ring A wherein Ring A is

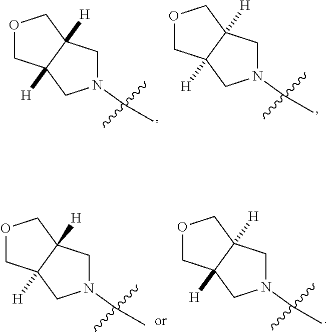 [SMILES: *N1C[C@H]2COC[C@H]2C1 ; *N1C[C@H]2COC[C@H]2C1 ; *N1C[C@H]2COC[C@@H]2C1 ; *N1C[C@@H]2COC[C@H]2C1]

In certain embodiments, provided herein are compounds of Formula (I), (Ia), (Ib), (Ic) or (Id) wherein Ring A

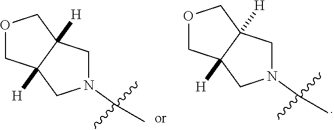 [SMILES: *N1C[C@H]2COC[C@H]2C1 ; *N1C[C@@H]2COC[C@H]2C1]

In certain embodiments, provided herein are compounds of Formula (I), (Ia), (Ib), (Ic) or (Id) wherein Ring A is a bridged heterocyclyl.

In certain embodiments, provided herein are compounds of Formula (I), (Ia), (Ib), (Ic) or (Id) wherein Ring A is

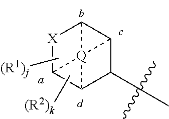

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and
wherein each R¹ and R² is as described for Formula (I), (Ia), (Ib), (Ic) or (Id), respectively. In certain embodiments, Q is —(CH₂)_{g}— attached to the ring carbon atoms at a and b, c and d, a and c, orb and d; g is 1, 2 or 3 and the remainder of X, R¹, R², j, k and t are as described for Formula (I), (Ia), (Ib), (Ic) or (Id).

In certain embodiments, provided herein are compounds of Formula (I), (Ia), (Ib), (Ic) or (Id) wherein Ring A is

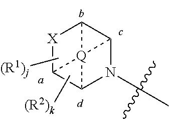

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2;
each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰ and R¹¹ are as described for Formula (I), (Ia), (Ib), (Ic) or (Id), respectively. In certain embodiments, Q is —(CH₂)_{g}— attached to the ring carbon atoms at a and b, c and d, a and c, or b and d; g is 1, 2 or 3 and the remainder of X, R¹, R², j, k and t are as described for Formula (I), (Ia), (Ib), (Ic) or (Id).

In certain embodiments, provided herein are compounds of Formula (I), (Ia), (Ib), (Ic) or (Id) wherein Ring A is

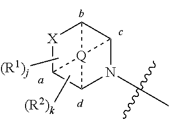

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2;
each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰ and R¹¹ are as described for Formula (I), (Ia), (Ib), (Ic) or (Id), respectively. In certain embodiments, Q is —(CH₂)_{g}— attached to the ring carbon atoms at a and b, c and d, a and c, orb and d; g is 1, 2 or 3 and the remainder of X, R¹, R², j, k and t are as described for Formula (I), (Ia), (Ib), (Ic) or (Id).

In certain embodiments, provided herein are compounds of Formula (I), (Ia), (Ib), (Ic) or (Id) wherein Ring A is

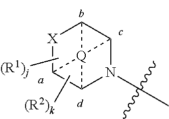

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2;
each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; and each R¹¹ is independently C₁₋₃alkyl or haloC₁₋₃alkyl. In certain embodiments, Q is —(CH₂)_{g}— attached to the ring carbon atoms at a and b, c and d, a and c, or b and d; g is 1, 2 or 3 and the remainder of X, R¹, R², j, k and t are as described for Formula (I), (Ia), (Ib), (Ic) or (Id).

In certain embodiments, provided herein are compounds of Formula (I), (Ia), (Ib), (Ic) or (Id) wherein Ring A is

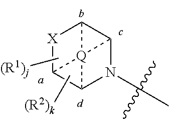

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2;
each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl. In certain embodiments, Q is —(CH₂)_{g}— attached to the ring carbon atoms at a and b, c and d, a and c, or b and d; g is 1, 2 or 3 and the remainder of X, R¹, R², j, k and t are as described for Formula (I), (Ia), (Ib), (Ic) or (Id).

In certain embodiments, provided herein are compounds of Formula (I), (Ia), (Ib), (Ic) or (Id) wherein Ring A is

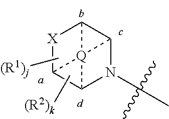

wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; R¹ and R² are —R^{u}OR⁶; R^{u} is a direct bond, methylene or ethylene and R⁶ is hydrogen or C₁₋₃alkyl; and j and k are each independently 0 or 1. In certain embodiments, Q is attached to the ring carbon atoms at a and b, a and c, or b and d. In certain embodiments, Q is —(CH₂)_{g}— attached to the ring carbon atoms at a and b, c and d, a and c, or b and d. In certain embodiments, X is —O—. In certain embodiments, Q is —(CH₂)_{g}— attached to the ring carbon atoms at a and b, a and c, or b and d. In certain embodiments, X is —O—.

In certain embodiments, provided herein are compounds of Formula (I), (Ia), (Ib), (Ic) or (Id) wherein Ring A is selected from:

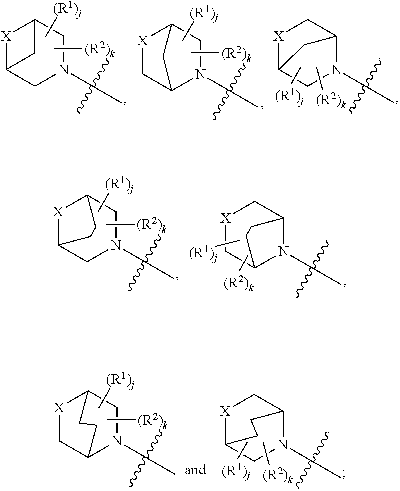

and X, R¹, R², j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

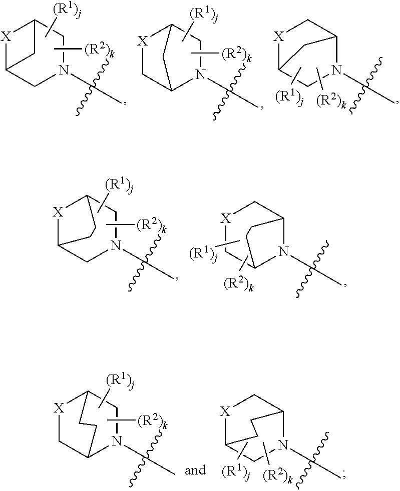

each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; R^{u}, R⁶, R⁷, R⁸, R⁹, R¹⁰ and X are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

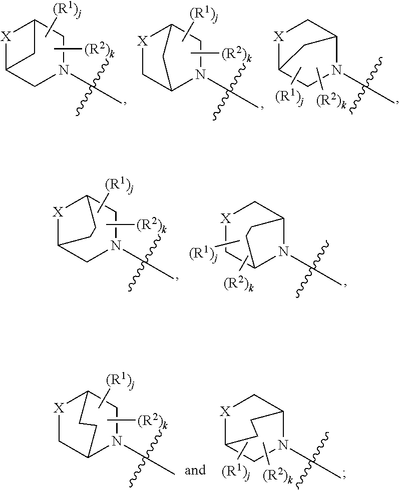

each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹ and X are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

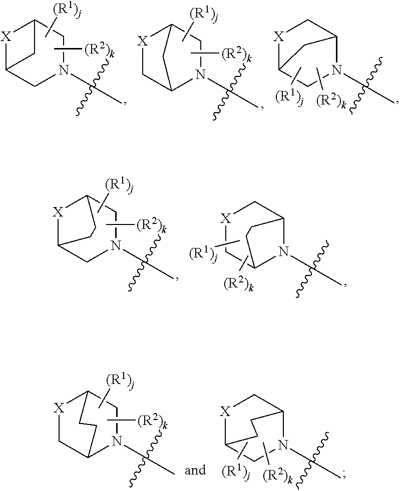

each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; and each R¹¹ is independently C₁₋₃alkyl or haloC₁₋₃alkyl and X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

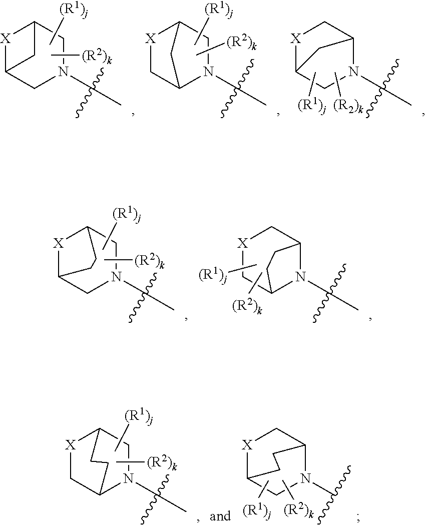

each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

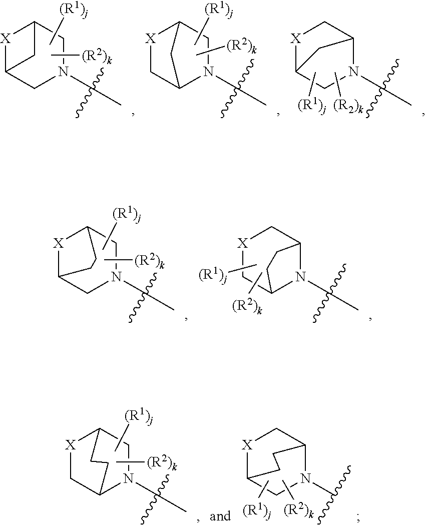

R¹ and R² are —R^{u}OR⁶; R^{u} is a direct bond, methylene or ethylene and R⁶ is hydrogen or C₁₋₃alkyl; j and k are each independently 0 or 1; and X is as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

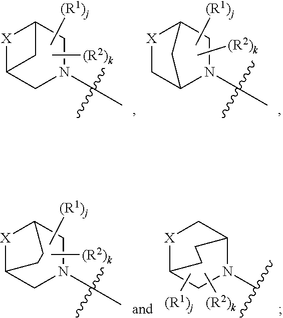

and X, R¹, R², j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

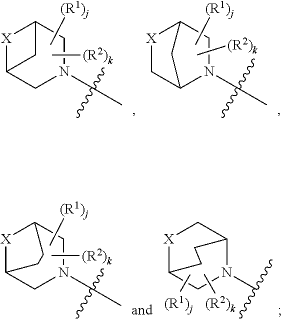

each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; R^{u}, R⁶, R⁷, R⁸, R⁹, R¹⁰ and X are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

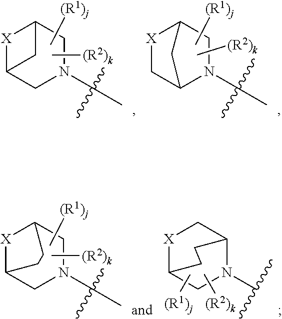

each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹¹ and X are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

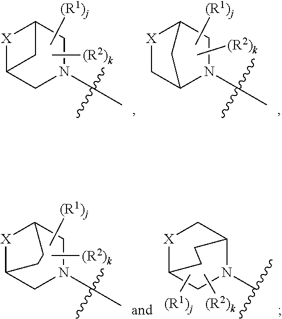

each R¹ is independently C₁₋₃alkyl, haloC₁₋₃alkyl, cyanoC₁₋₃alkyl, —R^{u}NR⁷R⁸, —R^{u}C(O)NR⁷R⁸ , —R^{u}C(O)R¹¹, —R^{u}NR¹⁰C(O)R¹¹ or —R^{u}NR¹⁰C(O)OR⁹; _{each R}² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; and each R¹¹ is independently C₁₋₃alkyl or haloC₁₋₃alkyl and X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

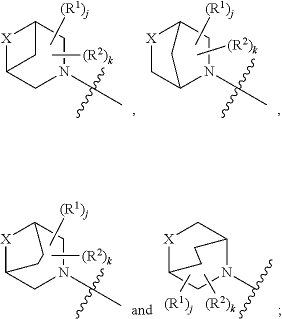

each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is selected from:

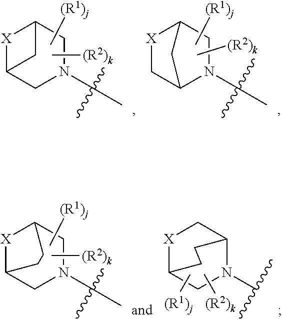

R¹ and R² are —R^{u}OR⁶; R^{u} is a direct bond, methylene or ethylene and R⁶ is hydrogen or C₁₋₃alkyl; j and k are each independently 0 or 1; and X is as described for Formula (I), (Ia), (Ib), (Ic) or (Id), respectively. In certain embodiments, X is —O—. In certain embodiments, provided herein are compounds of Formula (I), (Ia), (Ib), (Ic) or (Id) wherein Ring A is selected from:

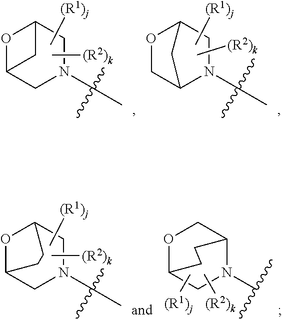

and R¹, R², j and k are as described for Formula (I), (Ia), (Ib), (Ic) or (Id), respectively.

In certain embodiments, provided herein are compounds of Formula (I), (Ia), (Ib), (Ic) or (Id) wherein Ring A is

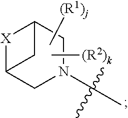

and X, R¹, R², j and k are as described for Formula (I), (Ia), (Ib), (Ic) or (Id), respectively. In certain embodiments, X is —O— and j and k are both 0.

In certain embodiments, provided herein are compounds of Formula (I), (Ia), (Ib), (Ic) or (Id) wherein Ring A is

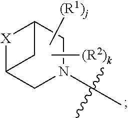

each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹ or —R^{u}NR¹⁰C(O)OR⁹; R^{u}, R⁶, R⁷, R⁸, R⁹, R¹⁰, X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is

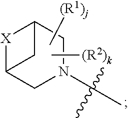

each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰, R¹, X, j and k are as described for Formula (I), (Ia), (Ib), (Ic) or (Id), respectively. In certain embodiments, X is —O—.

In certain embodiments, provided herein are compounds of Formula (I), (Ia), (Ib), (Ic) or (Id) wherein Ring A is

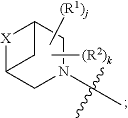

each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸ , —R^{u}C(O)R¹¹, —R^{u}NR¹⁰C(O)R¹¹ or —R^{u}NR¹⁰C(O)OR⁹; each R² is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; and each R¹¹ is independently C₁₋₃alkyl or haloC₁₋₃alkyl and X, j and k are as described for Formula (I), (Ia), (Ib), (Ic) or (Id), respectively. In certain embodiments, X is —O—. In certain embodiments, X is —O— and j and k are both 0.

In certain embodiments, provided herein are compounds of Formula (I), (Ia), (Ib), (Ic) or (Id) wherein Ring A is

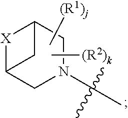

each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; and each R⁶ is hydrogen, C₁₋₃alkyl or haloC₁₋₃alkyl and X, j and k are as described for Formula (I), (Ia), (Ib), (Ic) or (Id), respectively. In certain embodiments, X is —O—. In certain embodiments, X is —O— and j and k are both 0.

In certain embodiments, provided herein are compounds of Formula (I), (Ia), (Ib), (Ic) or (Id) wherein Ring A is

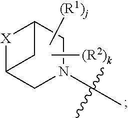

R¹ and R² are —R^{u}OR⁶; R^{u} is a direct bond, methylene or ethylene and R⁶ is hydrogen or C₁₋₃alkyl; j and k are each independently 0 or 1; and X is as described for Formula (I), (Ia), (Ib), (Ic) or (Id), respectively. In certain embodiments, X is —O—.

In certain embodiments, provided herein are compounds of Formula (II):

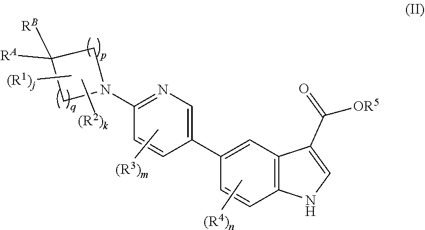

or a pharmaceutically acceptable salt, solvate, hydrate, a single stereoisomer, a mixture of stereoisomers or an isotopic variant thereof, wherein R^{A} and R^{B}, together with the carbon atom to which they are attached, form cycloalkyl or heterocyclyl;
p is 0, 1, 2 or 3;
q is 0, 1, 2 or 3, provided the sum of p and q are 2, 3, 4 or 5; and
wherein R¹, R², R³, R⁴, R⁵, j, k, m and n are as described for Formula (I) and R¹ and R² may be on either ring of the spirocycle.

In certain embodiment, provided herein are compounds of Formula (IIa):

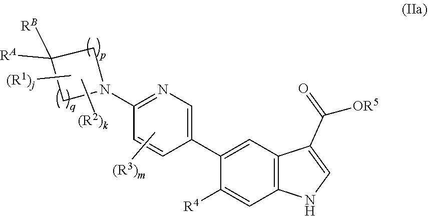

or a pharmaceutically acceptable salt, solvate, hydrate, a single stereoisomer, a mixture of stereoisomers or an isotopic variant thereof, wherein:
R^{A} and R^{B}, together with the carbon atom to which they are attached, form cycloalkyl or heterocyclyl;
p is 0, 1, 2 or 3;
q is 0, 1, 2 or 3, provided the sum of p and q are 2, 3, 4 or 5; and
wherein R¹, R², R³, R⁴, R⁵, j, k and m are as described for Formula (I) or Formula (Ia) and R¹ and R² may be on either ring of the spirocycle.

In certain embodiments, provided herein are compounds of Formula (IIb):

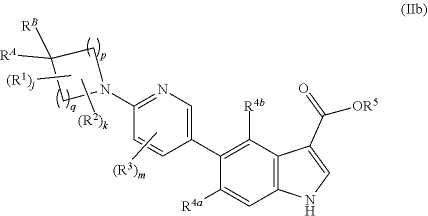

or a pharmaceutically acceptable salt, solvate, hydrate, a single stereoisomer, a mixture of stereoisomers or an isotopic variant thereof, wherein:
R^{A} and R^{B}, together with the carbon atom to which they are attached, form cycloalkyl or heterocyclyl;
p is 0, 1, 2 or 3;
q is 0, 1, 2 or 3, provided the sum of p and q are 2, 3, 4 or 5;
R^{4a} and R^{4b} are each independently halo, alkyl, haloalkyl or cyano; and
wherein R¹, R², R³, R⁵, j, k and m are as described for Formula (I) or Formula (Ib) elsewhere herein and R¹ and R² may be on either ring of the spirocycle.

In certain embodiments, provided herein are compounds of Formula (IIc):

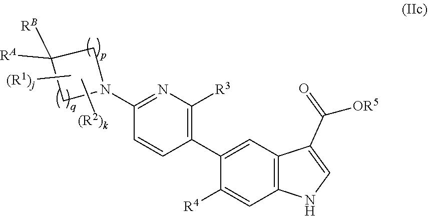

or a pharmaceutically acceptable salt, solvate, hydrate, a single stereoisomer, a mixture of stereoisomers or an isotopic variant thereof, wherein:
R^{A} and R^{B}, together with the carbon atom to which they are attached, form cycloalkyl or heterocyclyl;
p is 0, 1, 2 or 3; and
q is 0, 1, 2 or 3, provided the sum of p and q are 2, 3, 4 or 5;
wherein R¹, R², R³, R⁴, R⁵, j and k are as described for Formula (I) or Formula (Ic) elsewhere herein and R¹ and R² may be on either ring of the spirocycle.

In certain embodiments, provided herein are compounds of Formula (IId):

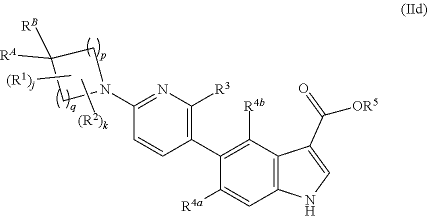

or a pharmaceutically acceptable salt, solvate, hydrate, a single stereoisomer, a mixture of stereoisomers or an isotopic variant thereof, wherein:
R^{A} and R^{B}, together with the carbon atom to which they are attached, form cycloalkyl or heterocyclyl;
p is 0, 1, 2 or 3; and
q is 0, 1, 2 or 3, provided the sum of p and q are 2, 3, 4 or 5;
wherein R¹, R², R³, R^{4a}, R^{4b}, R⁵, j and k are as described for Formula (I) or Formula (Id) elsewhere herein and R¹ and R² may be on either ring of the spirocycle.

In certain embodiments, provided herein are compounds of Formula (III):

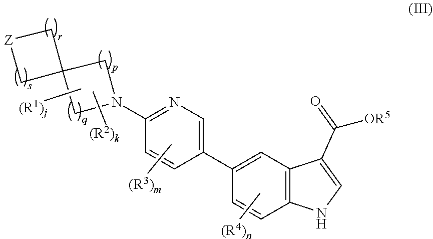

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2; and
wherein R¹, R², R³, R⁴, R⁵, j, k, m and n are as described for Formula (I) elsewhere herein and R¹ and R² may be on either ring of the spirocycle.

In certain embodiments, provided herein are compounds of Formula (IIIa):

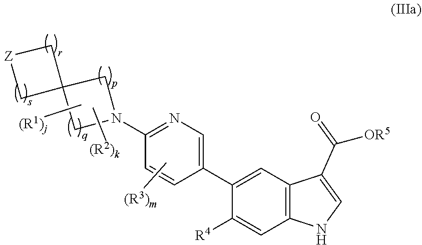

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2;
wherein R¹, R², R³, R⁴, R⁵, j, k and m are as described for Formula (I) or Formula (Ia) elsewhere herein and R¹ and R² may be on either ring of the spirocycle.

In certain embodiments, provided herein are compounds of Formula (IIIb):

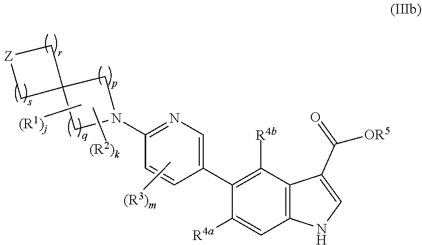

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5; and
t is 0, 1 or 2;
wherein R¹, R², R³, R^{4a}, R^{4b}, R⁵, j, k and m are as described for Formula (I) or Formula (Ib) elsewhere herein and R¹ and R² may be on either ring of the spirocycle.

In certain embodiments, provided herein are compounds of Formula (IIIc):

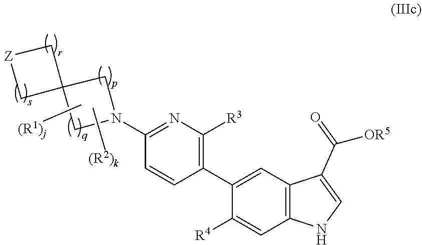

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2;
wherein R¹, R², R³, R⁴, R⁵, j and k are as described for Formula (I) or Formula (Ic) elsewhere herein and R¹ and R² may be on either ring of the spirocycle. In certain embodiments, r, s, p and q are all l and j and k are both 0.

In certain embodiments, provided herein are compounds of Formula (IIId):

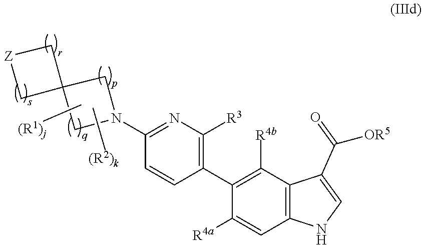

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2;
wherein R¹, R², R³, R^{4a}, R^{4b}, R⁵, j and k are as described for Formula (I) or Formula (Id) elsewhere herein and R¹ and R² may be on either ring of the spirocycle.

In certain embodiments, provided herein are compounds of Formula (IIIb) or (IIId) wherein Z is —O—, R^{4a} is halo and R^{4b} is halo, alkyl, haloalkyl or cyano, and p, q, r and s are all 1. In certain embodiments, provided herein are compounds of Formula (IIIb) or (IIId) wherein Z is —O—, R^{4a} and R^{4b} are both halo and p, q, r and s are all 1. In certain embodiments, provided herein are compounds of Formula (IIIb) or (IIId) wherein Z is —O—, R^{4a} is fluoro or chloro and R^{4b} is fluoro, chloro, C₁₋₃alkyl, haloC₁₋₃alkyl or cyano and p, q, r and s are all 1. In certain embodiments, provided herein are compounds of Formula (IIIb) or (IIId) wherein Z is —O—, R^{4a} and R^{4b} are both chloro and p, q, r and s are all 1.

In certain embodiments, provided herein are compounds of Formula (III), (IIIa) or (IIIc) wherein Z is —O—, R⁴ is halo, C₁₋₃alkyl, haloC₁₋₃alkyl or cyano, and p, q, r and s are all 1. In certain embodiments, provided herein are compounds of Formula (III), (IIIa) or (IIIc) wherein Z is —O—, R⁴ is halo and p, q, r and s are all 1. In certain embodiments, provided herein are compounds of Formula (III), (IIIa) or (IIIc) wherein Z is —O—, R⁴ is fluoro or chloro and p, q, r and s are all 1. In certain embodiments, provided herein are compounds of Formula (III), (IIIa) or (IIIc) wherein Z is —O—, R⁴ is chloro and p, q, r and s are all 1.

In certain embodiments, provided herein are compounds of Formula (IV):

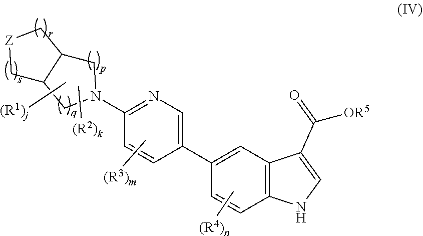

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and wherein R¹, R², R³, R⁴, R⁵, j, k, m and n are as described for Formula (I) elsewhere herein and R¹ and R² may be on either ring of the fused bicycle. In certain embodiments, Z is —O—. In certain embodiments, Z is —O— and j and k are both 0.

In certain embodiments, provided herein are compounds of Formula (IVa):

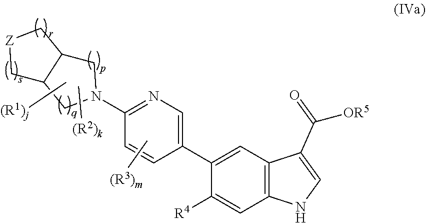

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and wherein R¹, R², R³, R⁴, R⁵, j, k, and m are as described for Formula (I) or Formula (Ia) elsewhere herein, and R¹ and R² may be on either ring of the fused bicycle. In certain embodiments, Z is —O—. In certain embodiments, Z is —O— and j and k are both 0.

In certain embodiments, provided herein are compounds of Formula (IVb):

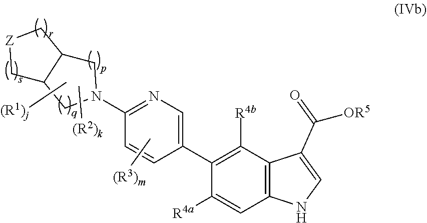

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—,
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and wherein R¹, R², R³, R^{4a}, R^{4b}, R⁵, j, k, and m are as described for Formula (I) or Formula (Ib) elsewhere herein, and R¹ and R² may be on either ring of the fused bicycle. In certain embodiments, Z is —O—. In certain embodiments, Z is —O— and j and k are both 0.

In certain embodiments, provided herein are compounds of Formula (IVc):

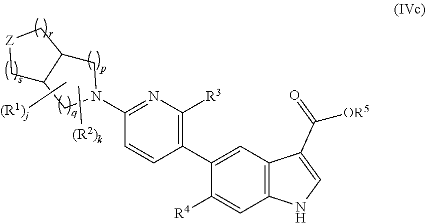

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—,
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and wherein R¹, R², R³, R⁴, R⁵, j, and k are as described for Formula (I) or Formula (Ic) elsewhere herein and R¹ and R² may be on either ring of the fused bicycle. In certain embodiments, Z is —O—. In certain embodiments, Z is —O— and j and k are both 0. In certain embodiments, Z is —O—. In certain embodiments, Z is —O—, p, q, r and s are all 1 and j and k are both 0.

In certain embodiments, provided herein are compounds of Formula (IVd):

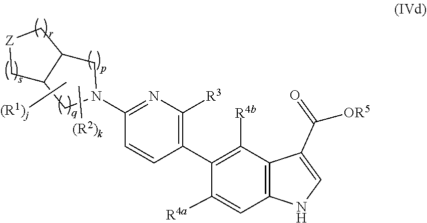

or a pharmaceutically acceptable salt, solvate, hydrate, a single stereoisomer, a mixture of stereoisomers or an isotopic variant thereof, wherein:
Z is —N(R^{C})—, —N(R^{G})S(O)ₜ—, —O—, —S(O)ₜ—, or —C(R^{E})(R^{F})—,
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and wherein R¹, R², R³, R^{4a}, R^{4b}, R⁵, j and k are as described for Formula (I) of Formula (Id) elsewhere herein, and R¹ and R² may be on either ring of the fused bicycle. In certain embodiments, Z is —O—. In certain embodiments, Z is —O— and j and k are both 0. In certain embodiments, Z is —O— and j and k are both 0.

In certain embodiments, provided herein are compounds of Formula (V):

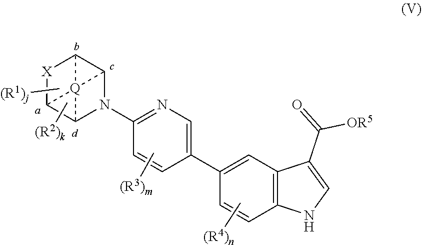

or a pharmaceutically acceptable salt, solvate, hydrate, a single stereoisomer, a mixture of stereoisomers or an isotopic variant thereof, wherein:
Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and wherein R¹, R², R³, R⁴, R⁵, j, k, m and n are as described for Formula (I). In certain embodiments, Q is attached to the ring carbon atoms at a and b, a and c or b and d. In certain embodiments, Q is —(CH₂)_{g}—, g is 1, 2 or 3 and is attached to the ring carbon atoms at a and b, a and c or b and d.

In certain embodiments, provided herein are compounds of Formula (Va):

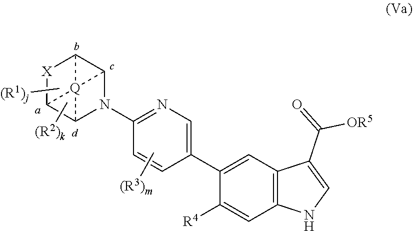

or a pharmaceutically acceptable salt, solvate, hydrate, a single stereoisomer, a mixture of stereoisomers or an isotopic variant thereof, wherein:
Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and wherein R¹, R², R³, R⁴, R⁵, j, k and m are as described for Formula (I) or Formula (Ia) elsewhere herein. In certain embodiments, Q is attached to the ring carbon atoms at a and b, a and c or b and d. In certain embodiments, Q is —(CH₂)_{g}—, g is 1, 2 or 3 and is attached to the ring carbon atoms at a and b, a and c or b and d.

In certain embodiments, provided herein are compounds of Formula (Vb):

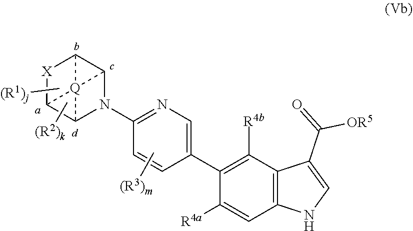

or a pharmaceutically acceptable salt, solvate, hydrate, a single stereoisomer, a mixture of stereoisomers or an isotopic variant thereof, wherein:
Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and wherein R¹, R², R³, R^{4a}, R^{4b}, R⁵, j, k and m are as described for Formula (I) or Formula (Ib) elsewhere herein. In certain embodiments, Q is attached to the ring carbon atoms at a and b, a and c or b and d. In certain embodiments, Q is —(CH₂)_{g}—, g is 1, 2 or 3 and is attached to the ring carbon atoms at a and b, a and c or b and d.

In certain embodiments, provided herein are compounds of Formula (Vc):

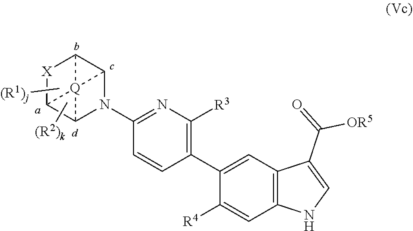

or a pharmaceutically acceptable salt, solvate, hydrate, a single stereoisomer, a mixture of stereoisomers or an isotopic variant thereof, wherein:
Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and wherein R¹, R², R³, R⁴, R⁵, j and k are as described for Formula (I) or Formula (Ic) elsewhere herein. In certain embodiments, Q is attached to the ring carbon atoms at a and b, a and c or b and d. In certain embodiments, Q is —(CH₂)_{g}—, g is 1, 2 or 3 and is attached to the ring carbon atoms at a and b, a and c or b and d. In certain embodiments, Q is —(CH₂)_{g}—, g is 1, 2 or 3 and is attached to the ring carbon atoms at a and b and j and k are both 0. In certain embodiments, X is —O—, Q is —(CH₂)_{g}—, g is 1, 2 or 3 and is attached to the ring carbon atoms at a and b; and j and k are both 0.

In certain embodiments, provided herein are compounds of Formula (Vd):

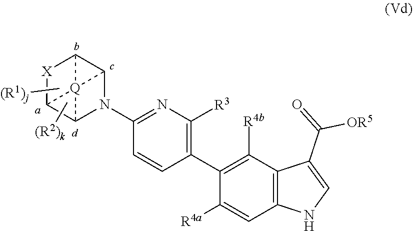

or a pharmaceutically acceptable salt, solvate, hydrate, a single stereoisomer, a mixture of stereoisomers or an isotopic variant thereof, wherein:
Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})-, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and wherein R¹, R², R³, R^{4a}, R^{4b} and R⁵ are as described for Formula (I) or Formula (Id) elsewhere herein. In certain embodiments, Q is attached to the ring carbon atoms at a and b, a and c or b and d. In certain embodiments, Q is —(CH₂)_{g}—, g is 1, 2 or 3 and is attached to the ring carbon atoms at a and b, a and c or b and d.

In certain embodiments, provided herein are compounds of Formula (Ib), (Id), (IIb), (IId), (IIIb), (IIId), (IVb), (IVd), (Vb) or (Vd), wherein R^{4a} is halo and R^{4b} is halo, alkyl, haloalkyl or cyano. In certain embodiments, provided herein are compounds of Formula (Ib), (Id), (IIb), (IId), (IIIb), (IIId), (IVb), (IVd), (Vb) or (Vd), wherein R^{4a} and R^{4b} are both halo. In certain embodiments, provided herein are compounds of Formula (Ib), (Id), (IIb), (IId), (IIIb), (IIId), (IVb), (IVd), (Vb) or (Vd), wherein R^{4a} and R^{4b} are both chloro. In certain embodiments, provided herein are compounds of Formula (Ib), (Id), (IIb), (IId), (IIIb), (IIId), (IVb), (IVd), (Vb) or (Vd), wherein R^{4a} is fluoro or chloro and R^{4b} is fluoro, chloro, C₁₋₃alkyl, haloC₁₋₃alkyl or cyano.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), (IVb), (IVc), (IVd), (V), (Va), (Vb), (Vc) or (Vd), wherein each R¹ is independently alkyl, haloalkyl, cyanoalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)OR⁹—, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰ and R¹¹ are as described for Formula (I), (Ia), (Ib), (Ic), (Id), (II), (IIa), (IIb), (IIc), (IId), (III), (IIIa), (IIIb), (IIIc), (IIId), (IV), (IVa), (IVb), (IVc), (IVd), (V), (Va), (Vb), (Vc) or (Vd), respectively, elsewhere herein. In certain embodiments, R³ is methoxy or ethoxy. In certain embodiments, R³ is methoxy. In certain embodiments, R⁴ or R^{4a} is fluoro or chloro. In yet certain embodiments, R⁴ or R^{4a} is chloro. In certain embodiments, R^{4b} is fluoro or chloro. In yet certain embodiments, R^{4b} is chloro.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein each R¹ is independently alkyl, haloalkyl, cyanoalkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰C(O)OR⁹; each R² is independently halo, oxo, cyano, alkyl, haloalkyl, cyanoalkyl, —R^{u}OR⁶, —R^{u}NR⁷R⁸, —R^{u}C(O)NR⁷R⁸, —R^{u}C(O)R¹¹ or —R^{u}NR¹⁰C(O)OR⁹; and R^{u}, R⁶, R⁷, R⁸, R⁹, R¹⁰ and R¹¹ are as described for Formula (I), (Ia), (Ib), (Ic) or (Id) respectively, elsewhere herein. In certain embodiments, R³ is methoxy or ethoxy. In certain embodiments, R³ is methoxy. In certain embodiments, R⁴ or R^{4a} is fluoro or chloro. In yet certain embodiments, R⁴ or R^{4a} is chloro. In certain embodiments, R^{4b} is fluoro or chloro. In yet certain embodiments, R^{4b} is chloro.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein each R¹ is independently C₁₋₃alkyl, haloC₁₋₃alkyl, cyanoC₁₋₃alkyl, —R^{u}OR⁶, —R^{u}NR⁷R⁸, —R^{u}C(O)NR⁷R⁸, —R^{u}C(O)R¹¹, —R^{u}NR¹⁰C(O)R¹¹ or —R^{u}NR¹⁰CO(O)OR⁹; each R¹ is independently halo, oxo, cyano, C₁₋₃alkyl, haloC₁₋₃alkyl, cyanoC₁₋₃alkyl, —R^{u}OR⁶ or —R^{u}NR⁷R⁸; each R^{u} is independently a direct bond, methylene or ethylene; each R⁶, R⁷, R⁸ and R¹⁰ are each independently hydrogen, C₁₋₃alkyl, haloC₁₋₃alkyl or cyanoC₁₋₃alkyl; and each R⁹ is independently hydrogen, C₁₋₄alkyl, haloC₁₋₄alkyl or cyanoC₁₋₄alkyl; each R¹¹ is independently C₁₋₃alkyl or haloC₁₋₃alkyl. In certain embodiments, R³ is methoxy or ethoxy. In certain embodiments, R³ is methoxy. In certain embodiments, R⁴ or R^{4a} is fluoro or chloro. In yet certain embodiments, R⁴ or R^{4a} is chloro. In certain embodiments, R^{4b} is fluoro or chloro. In yet certain embodiments, R^{4b} is chloro.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein each R¹ is independently C₁₋₃alkyl, haloC₁₋₃alkyl, cyanoC₁₋₃alkyl or —R^{u}OR⁶; each R² is independently oxo, cyano, C₁₋₃alkyl, haloC₁₋₃alkyl, cyanoC₁₋₃alkyl or —R^{u}OR⁶; each R^{u} is independently a direct bond, methylene or ethylene; each R⁶ is hydrogen, C₁₋₃alkyl or haloC₁₋₃alkyl. In certain embodiments, R³ is methoxy or ethoxy. In certain embodiments, R³ is methoxy. In certain embodiments, R⁴ or R^{4a} is fluoro or chloro. In yet certain embodiments, R⁴ or R^{4a} is chloro. In certain embodiments, R^{4b} is fluoro or chloro. In yet certain embodiments, R^{4b} is chloro.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein j and k are both 0 and the other variables are as described elsewhere herein for Formula (I), (Ia), (Ib), (Ic), (Id), (II), (IIa), (IIb), (IIc), (IId), (III), (IIIa), (IIIb), (IIIc), (IIId), (IV), (IVa), respectively.

In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein j and k are both 1 and the other variables are as described elsewhere herein for Formula (I), (Ia), (Ib), (Ic), (Id), (II), (IIa), (IIb), (IIc), (IId), (III), (IIIa), (IIIb), (IIIc), (IIId), (IV), (IVa), respectively. In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein the sum of j and k is 1 and the other variables are as described elsewhere herein for Formula (I), (Ia), (Ib), (Ic), (Id), (II), (IIa), (IIb), (IIc), (IId), (III), (IIIa), (IIIb), (IIIc), (IIId), (IV), (IVa), respectively. In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein j is 0 and k is 1 and the other variables are as described elsewhere herein for Formula (I), (Ia), (Ib), (Ic), (Id), (II), (IIa), (IIb), (IIc), (IId), (III), (IIIa), (IIIb), (IIIc), (IIId), (IV), (IVa), respectively. In certain embodiments, provided herein are compounds of Formula (I), (Ia), (Ib), (Ic), (Id), (II), (IIa), (IIb), (IIc), (IId), (III), (IIIa), (IIIb), (IIIc), (IIId), (IV), (IVa), wherein j is 1 and k is 0 and the other variables are as described elsewhere herein for Formula (I), (Ia), (Ib), (Ic), (Id), (II), (IIa), (IIb), (IIc), (IId), (III), (IIIa), (IIIb), (IIIc), (IIId), (IV), (IVa), respectively. In certain embodiments, R³ is methoxy or ethoxy. In certain embodiments, R³ is methoxy. In certain embodiments, R⁴ or R^{4a} is fluoro or chloro. In yet certain embodiments, R⁴ or R^{4a} is chloro. In certain embodiments, R^{4b} is fluoro or chloro. In yet certain embodiments, R^{4b} is chloro.

In certain embodiments, provided herein is a compound of Formula (I) wherein the compound is selected from:
6-chloro-5-(2-methoxy-6-(2-oxa-6-azaspiro[3.3]heptan-6-yl)pyridin-3-yl)-1H-indole-3-carboxylic acid; [Ex 1]
5-(6-((1S, 4S)2-oxa-5-azabicyclo[2.2.1]heptan-5-yl)-2-methoxypyridin-3-yl)-6-chloro-1H-indole-3-carboxylic acid;
5-(6-((1R, 4R)2-oxa-5-azabicyclo[2.2.1]heptan-5-yl)-2-methoxypyridin-3-yl)-6-chloro-1H-indole-3-carboxylic acid;
5-(6-(2-oxa-5-azabicyclo[2.2.1]heptan-5-yl)-2-methoxypyridin-3-yl)-6-chloro-1H-indole-3-carboxylic acid; [Ex 2]
6-chloro-5-(6-(6-(hydroxymethyl)-3-azabicyclo[3.1.0]hexan-3-yl)-2-methoxypyridin-3-yl)-1H-indole-3-carboxylic acid;
6-chloro-5-(6-((trans)-6-(hydroxymethyl)-3-azabicyclo[3.1.0]hexan-3-yl)-2-methoxypyridin-3-yl)-1H-indole-3-carboxylic acid; [Ex 3]
6-chloro-5-(6-((cis)-6-(hydroxymethyl)-3-azabicyclo[3.1.0]hexan-3-yl)-2-methoxypyridin-3-yl)-1H-indole-3-carboxylic acid;
6-chloro-5-(6-(6-hydroxy-2-azaspiro[3.3]heptan-2-yl)-2-methoxypyridin-3-yl)-1H-indole-3-carboxylic acid; [Ex 4]
6-chloro-5-(2-methoxy-6-(2-oxa-6-azaspiro[3.4]octan-6-yl)pyridin-3-yl)-1H-indole-3-carboxylic acid; [Ex 5]
6-chloro-5-(2-methoxy-6-(2,6-diazaspiro[3.3]heptan-2-yl)pyridin-3-yl)-1H-indole-3-carboxylic acid; [Ex 6]
5-(6-(6-((tert-butoxycarbonyl)amino)-2-azaspiro[3.3]heptan-2-yl)-2-methoxypyridin-3-yl)-6-chloro-1H-indole-3-carboxylic acid; [Ex 7]
6-chloro-5-(2-methoxy-6-(5-oxa-2-azaspiro[3.4]octan-2-yl)pyridin-3-yl)-1H-indole-3-carboxylic acid; [Ex 8]
6-chloro-5-(2-methoxy-6-(6-(methoxymethyl)-3-azabicyclo[3.1.0]hexan-3-yl)pyridin-3-yl)-1H-indole-3-carboxylic acid;
6-chloro-5-(2-methoxy-6-((cis)-6-(methoxymethyl)-3-azabicyclo[3.1.0]hexan-3-yl) pyridin-3-yl)-1H-indole-3-carboxylic acid;
6-chloro-5-(2-methoxy-6-((trans)-6-(methoxymethyl)-3-azabicyclo[3.1.0]hexan-3-yl) pyridin-3-yl)-1H-indole-3-carboxylic acid; [Ex 9]
5-(6-(6-amino-2-azaspiro[3.3]heptan-2-yl)-2-methoxypyridin-3-yl)-6-chloro-1H-indole-3-carboxylic acid; [Ex 10]
6-chloro-5-(2-methoxy-6-(2,6-diazaspiro[3.3]heptan-2-yl)pyridin-3-yl)-1H-indole-3-carboxylic acid; [Ex 11]
5-[6-(5-azaspiro[2.3]hexan-5-yl)-2-methoxy-3-pyridyl]-6-chloro-1H-indole-3-carboxylic acid; [Ex 12]
6-chloro-5-(2-methoxy-6-(2-azaspiro[3.3]heptan-2-yl)pyridin-3-yl)-1H-indole-3-carboxylic acid; [Ex 13]
5-(6-(3-azabicyclo[3.1.0]hexan-3-yl)-2-methoxypyridin-3-yl)-6-chloro-1H-indole-3-carboxylic acid; [Ex 14]
6-chloro-5-(6-(1-cyano-3-azabicyclo[3.1.0]hexan-3-yl)-2-methoxypyridin-3-yl)-1H-indole-3-carboxylic acid; [Ex 15]
6-chloro-5-(6-((1R)1-cyano-3-azabicyclo[3.1.0]hexan-3-yl)-2-methoxypyridin-3-yl)-1H-indole-3-carboxylic acid;
6-chloro-5-(6-((1S)1-cyano-3-azabicyclo[3.1.0]hexan-3-yl)-2-methoxypyridin-3-yl)-1H-indole-3-carboxylic acid;
6-chloro-5-(6-(1,1-dioxido-1-thia-6-azaspiro[3.3]heptan-6-yl)-2-methoxypyridin-3-yl)-1H-indole-3-carboxylic acid; [Ex 16]
6-chloro-5-(2-methoxy-6-(6-oxo-2,5-diazaspiro[3.4]octan-2-yl)pyridin-3-yl)-1H-indole-3-carboxylic acid; [Ex 17]
6-chloro-5-(2-methoxy-6-(5-oxo-2,6-diazaspiro[3.4]octan-2-yl)pyridin-3-yl)-1H-indole-3-carboxylic acid; [Ex 18]
6-chloro-5-(2-methoxy-6-(7-oxo-2,6-diazaspiro[3.4]octan-2-yl)pyridin-3-yl)-1H-indole-3-carboxylic acid; [Ex 19]
5-(6-(6-oxa-3-azabicyclo[3.2.1]octan-3-yl)-2-methoxypyridin-3-yl)-6-chloro-1H-indole-3-carboxylic acid; [Ex 20]
5-(6-(1R,5S)-(6-oxa-3-azabicyclo[3.2.1]octan-3-yl)-2-methoxypyridin-3-yl)-6-chloro-1H-indole-3-carboxylic acid;
5-(6-(1S,5R)-(6-oxa-3-azabicyclo[3.2.1]octan-3-yl)-2-methoxypyridin-3-yl)-6-chloro-1H-indole-3-carboxylic acid;
6-chloro-5-(2-methoxy-6-(6-oxa-2-azaspiro[3.4]octan-2-yl)pyridin-3-yl)-1H-indole-3-carboxylic acid; [Ex 21]
6-chloro-5-(2-methoxy-6-(6-methyl-7-oxo-2,6-diazaspiro[3.4]octan-2-yl)pyridine-3-yl)-1H-indole-3-carboxylic acid; [Ex 22]
6-chloro-5-(2-methoxy-6-(6-methoxy-3-azabicyclo[3.1.0]hexan-3-yl)pyridin-3-yl)-1H-indole-3-carboxylic acid;
6-chloro-5-(2-methoxy-6-((cis)-6-methoxy-3-azabicyclo[3.1.0]hexan-3-yl)pyridine-3-yl)-1H-indole-3-carboxylic acid;
6-chloro-5-(2-methoxy-6-((trans)-6-methoxy-3-azabicyclo[3.1.0]hexan-3-yl)pyridin-3-yl)-1H-indole-3-carboxylic acid; [Ex 23]
6-chloro-5-(6-(6-hydroxy-3-azabicyclo[3.1.0]hexan-3-yl)-2-methoxypyridin-3-yl)-1H-indole-3-carboxylic acid;
6-chloro-5-(6-((cis)-6-hydroxy-3-azabicyclo[3.1.0]hexan-3-yl)-2-methoxypyridin-3-yl)-1H-indole-3-carboxylic acid;
6-chloro-5-(6-((trans)-6-hydroxy-3-azabicyclo[3.1.0]hexan-3-yl)-2-methoxypyridin-3-yl)-1H-indole-3-carboxylic acid; [Ex 24]
6-chloro-5-(6-(5-hydroxy-2-azaspiro[3.3]heptan-2-yl)-2-methoxypyridin-3-yl)-1H-indole-3-carboxylic acid; [Ex 25]
(R)-6-chloro-5-(6-(5-hydroxy-2-azaspiro[3.3]heptan-2-yl)-2-methoxypyridin-3-yl)-1H-indole-3-carboxylic acid;
(S)-6-chloro-5-(6-(5-hydroxy-2-azaspiro[3.3]heptan-2-yl)-2-methoxypyridin-3-yl)-1H-indole-3-carboxylic acid;
6-chloro-5-(6-(1-hydroxy-3-azabicyclo[3.1.0]hexan-3-yl)-2-methoxypyridin-3-yl)-1H-indole-3-carboxylic acid; [Ex 26]
(R)-6-chloro-5-(6-(1-hydroxy-3-azabicyclo[3.1.0]hexan-3-yl)-2-methoxypyridin-3-yl)-1H-indole-3-carboxylic acid;
(S)-6-chloro-5-(6-(1-hydroxy-3-azabicyclo[3.1.0]hexan-3-yl)-2-methoxypyridin-3-yl)-1H-indole-3-carboxylic acid;
6-chloro-5-(2-methoxy-6-((trans)-tetrahydro-1H-furo[3,4-c]pyrrol-5(3H)-yl)pyridine-3-yl)-1H-indole-3-carboxylic acid;
6-chloro-5-(2-methoxy-6-((cis)-tetrahydro-1H-furo[3,4-c]pyrrol-5(3H)-yl)pyridine-3-yl)-1H-indole-3-carboxylic acid; [Ex 27]
6-chloro-5-(6-(hexahydro-5H-furo[2,3-c]pyrrol-5-yl)-2-methoxypyridin-3-yl)-1H-indole-3-carboxylic acid; [Ex 28]
6-chloro-5-(6-((3aR,6aR)-hexahydro-5H-furo[2,3-c]pyrrol-5-yl)-2-methoxypyridin-3-yl)-1H-indole-3-carboxylic acid;
6-chloro-5-(6-((3aS,6aS)-hexahydro-5H-furo[2,3-c]pyrrol-5-yl)-2-methoxypyridin-3-yl)-1H-indole-3-carboxylic acid;
6-chloro-5-(6-((3aR,6aS)-hexahydro-5H-furo[2,3-c]pyrrol-5-yl)-2-methoxypyridin-3-yl)-1H-indole-3-carboxylic acid;
6-chloro-5-(6-((3aS,6aR)-hexahydro-5H-furo[2,3-c]pyrrol-5-yl)-2-methoxypyridin-3-yl)-1H-indole-3-carboxylic acid;
5-(6-(8-oxa-3-azabicyclo[3.2.1]octan-3-yl)-2-methoxypyridin-3-yl)-6-chloro-1H-indole-3-carboxylic acid; [Ex 29]
5-(6-((1R,5S)-8-oxa-3-azabicyclo[3.2.1]octan-3-yl)-2-methoxypyridin-3-yl)-6-chloro-1H-indole-3-carboxylic acid;
5-(6-((1S,5R)-8-oxa-3-azabicyclo[3.2.1]octan-3-yl)-2-methoxypyridin-3-yl)-6-chloro-1H-indole-3-carboxylic acid;
5-(6-(2-oxa-5-azabicyclo[2.2.2]octan-5-yl)-2-methoxypyridin-3-yl)-6-chloro-1H-indole-3-carboxylic acid; [Ex 30]
5-(6-((1R,4R)-2-oxa-5-azabicyclo[2.2.2]octan-5-yl)-2-methoxypyridin-3-yl)-6-chloro-1H-indole-3-carboxylic acid;
5-(6-((1S,4S)-2-oxa-5-azabicyclo[2.2.2]octan-5-yl)-2-methoxypyridin-3-yl)-6-chloro-1H-indole-3-carboxylic acid;
6-chloro-5-(2-methoxy-6-(1-oxa-7-azaspiro[3.5]nonan-7-yl)pyridin-3-yl)-1H-indole-3-carboxylic acid; [Ex 31]
6-chloro-5-(2-methoxy-6-(tetrahydro-1H-furo[3,4-c]pyrrol-5(3H)-yl)pyridin-3-yl)-1H-indole-3-carboxylic acid;
6-chloro-5-(2-methoxy-6-((trans)-tetrahydro-1H-furo[3,4-c]pyrrol-5(3H)-yl)pyridine-3-yl)-1H-indole-3-carboxylic acid; [Ex 32]
6-chloro-5-(2-methoxy-6-((3aR, 6aR)-tetrahydro-1H-furo[3,4-c]pyrrol-5(3H)-yl)pyridine-3-yl)-1H-indole-3-carboxylic acid; [Ex 33]
6-chloro-5-(2-methoxy-6-((3aS, 6aS)-tetrahydro-1H-furo[3,4-c]pyrrol-5(3H)-yl)pyridin-3-yl)-1H-indole-3-carboxylic acid; [Ex 34]
6-chloro-5-(2-methoxy-6-[8-oxa-2-azaspiro[4.5]decan-2-yl]pyridin-3-yl)-1H-indole-3-carboxylic acid; [Ex 35]
6-chloro-5-(2-methoxy-6-(7-oxa-2-azaspiro[3.5]nonan-2-yl)pyridin-3-yl)-1H-indole-3-carboxylic acid; [Ex 36]
6-chloro-5-(2-methoxy-6-(2-oxa-7-azaspiro[4.4]nonan-7-yl)pyridin-3-yl)-1H-indole-3-carboxylic acid; [Ex 37]
5-(6-(5-acetylhexahydropyrrolo[3,4-c]pyrrol-2(1H)-yl)-2-methoxypyridin-3-yl)-6-chloro-1H-indole-3-carboxylic acid;
5-(6-((3aR, 6aS)-5-acetylhexahydropyrrolo[3,4-c]pyrrol-2(1H)-yl)-2-methoxypyridin-3-yl)-6-chloro-1H-indole-3-carboxylic acid; [Ex 38]
5-(6-((3aS,6aR)-5-acetylhexahydropyrrolo[3,4-c]pyrrol-2(1H)-yl)-2-methoxypyridin-3-yl)-6-chloro-1H-indole-3-carboxylic acid;
5-(6-((3aR, 6aR)-5-acetylhexahydropyrrolo[3,4-c]pyrrol-2(1H)-yl)-2-methoxypyridin-3-yl)-6-chloro-1H-indole-3-carboxylic acid;
5-(6-((3aS,6aS)-5-acetylhexahydropyrrolo[3,4-c]pyrrol-2(1H)-yl)-2-methoxypyridin-3-yl)-6-chloro-1H-indole-3-carboxylic acid;
5-(6-(6-oxa-3-azabicyclo[3.1.1]heptan-3-yl)-2-methoxypyridin-3-yl)-6-chloro-1H-indole-3-carboxylic acid; [Ex 39]
6-fluoro-5-(2-methoxy-6-[2-oxa-6-azaspiro[3.3]heptan-6-yl]pyridin-3-yl)-1H-indole-3-carboxylic acid; [Ex 40] and
4,6-difluoro-5-(2-methoxy-6-(2-oxa-6-azaspiro[3.3]heptan-6-yl)pyridin-3-yl)-1H-indole-3-carboxylic acid. [Ex 41]

In certain embodiments, provided herein are isotopically enriched analogs of the compounds disclosed herein, for example, deuterated analogs, to improve pharmacokinetics (PK), pharmacodynamics (PD) and toxicity profiles of the compounds.

In certain embodiments, provided herein are pharmaceutical compositions comprising a compound of Formula (I), (Ia), (Ib), (Ic), (Id), (II), (IIa), (IIb), (IIc), (IId), (III), (IIIa), (IIIb), (IIIc), (IIId), (IV) or (IVa) and a pharmaceutically acceptable carrier.

Any combination of the groups described above for the various variables is contemplated herein. Throughout the specification, groups and substituents thereof are chosen by one skilled in the field to provide stable moieties and compounds.

The compounds of the present disclosure include the compounds themselves, as well as their salts, solvate and solvate of the salt, if applicable. Salts for the purposes of the present disclosure are preferably pharmaceutically acceptable salts of the compounds according to the present disclosure. Salts which are not themselves suitable for pharmaceutical uses but can be used, for example, for isolation or purification of the compounds according to the disclosure are also included. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion.

As used herein, “pharmaceutically acceptable salts” refer to acid or base addition salts, including but not limited to, base addition salts formed by the compound of Formula (I) having an acidic moiety with pharmaceutically acceptable cations, for example, sodium, potassium, magnesium, calcium, aluminum, lithium, and ammonium. Lists of suitable salts may be found in Remington's Pharmaceutical Sciences, 17th ed., Mack Publishing Company, Easton, Pa., 1985, p. 1418; S. M. Berge et al., “Pharmaceutical Salts”, J. Pharm. Sci. 1977, 66, 1-19; and “Pharmaceutical Salts: Properties, Selection, and Use. A Handbook”; Wermuth, C. G. and Stahl, P. H. (eds.) Verlag Helvetica Chimica Acta, Zurich, 2002 [ISBN 3-906390-26-8]; each of which is incorporated herein by reference in its entirety.

Solvates in the context of the present disclosure are designated as those forms of the compounds according to the present disclosure which form a complex in the solid or liquid state by stoichiometric coordination with solvent molecules. Hydrates are a specific form of solvates, in which the coordination takes place with water. The formation of solvates is described in greater detail in “Solvents and Solvent Effects in Organic Chemistry”; Reichardt, C. and Welton T.; John Wiley & Sons, 2011 [ISBN: 978-3-527-32473-6], the contents of which is incorporated herein by reference in its entirety.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

### Formulation

The term “pharmaceutical composition” as used herein is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt, or solvate or solvate of the salt thereof, and a pharmaceutically acceptable carrier.

The term “pharmaceutically acceptable carrier” refers to a carrier or an adjuvant that may be administered to a patient, together with a compound of the present disclosure, or a pharmaceutically acceptable salt, solvate or salt of the solvate, and which does not destroy the pharmacological activity thereof and is nontoxic when administered in doses sufficient to deliver a therapeutic amount of the compound.

The amount administered depends on the compound formulation, route of administration, etc. and is generally empirically determined, and variations will necessarily occur depending on the target, the host, and the route of administration, etc. Generally, the quantity of active compound in a unit dose of preparation may be varied or adjusted from about 1 milligram (mg) to about 100 mg or from about 1 mg to about 1000 mg, according to the particular application. For convenience, the total daily dosage may be divided and administered in portions during the day.

Solid dosage forms of the instant pharmaceutical compositions for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof In the case of capsules, tablets and pills, the dosage form may also comprise buffering agents.

Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the instant pharmaceutical compositions of tablets, dragées, capsules, pills, and granules can be prepared with coatings and shells such as enteric coatings and other pharmaceutical coatings. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Liquid dosage forms of the instant pharmaceutical compositions for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions of the instant compounds, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Pharmaceutical compositions of the present disclosure for injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Besides inert diluents, these pharmaceutical compositions may also contain adjuvants such as preservative, wetting agents, emulsifying agents, dispersing agents, sweetening, flavoring, and perfuming agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin. The compounds can be incorporated into slow release or targeted delivery systems such as polymer matrices, liposomes, and microspheres. Such formulations may provide more effective distribution of the compounds.

The pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable medium prior to use.

Dosage forms for topical administration of a compound or pharmaceutical composition of the present disclosure include powders, patches, sprays, ointments and inhalants. The active compound is mixed under sterile conditions with a pharmaceutically acceptable carrier and any preservatives, buffers, or propellants which may be required.

The compounds and compositions described herein can, for example, be administered orally, parenterally (e.g., subcutaneously, intracutaneously, intravenously or intramuscularly), topically, rectally, nasally sublingually or buccally, with a dosage ranging from about 0.01 milligrams per kilogram (mg/kg) to about 1000 mg/kg, (e.g., from about 0.01 to about 100 mg/kg, from about 0.1 to about 100 mg/kg) every 4 to 120 hours, or according to the requirements of the particular drug, dosage form, and/or route of administration. Other routes of administration include enteric, intraarterial, intraperitoneal and intrathecal administration. The interrelationship of dosages for animals and humans (based on milligrams per meter squared of body surface) is described by Freireich et al., Cancer Chemother. Rep. 50, 219-244 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or by injection. The methods herein contemplate administration of an effective amount of compound or compound composition to achieve a desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, and the judgment of the treating physician.

Dosage forms include from about 0.001 mg to about 2,000 mg (including, from about 0.001 mg to about 1,000 mg, from about 0.001 mg to about 500 mg, from about 0.01 mg to about 250 mg) of a compound of Formula (I) or a salt (e.g., a pharmaceutically acceptable salt) thereof as defined anywhere herein. The dosage forms can further include a pharmaceutically acceptable carrier and/or an additional therapeutic agent.

Appropriate dosage levels may be determined by any suitable method. Preferably, the active substance is administered at a frequency of 1 to 4 times per day for topical administration, or less often if a drug delivery system is used. Nevertheless, actual dosage levels and time course of administration of the active ingredients in the pharmaceutical compositions of the present disclosure may be varied so as to obtain an amount of the active ingredient which is effective to achieve a desired therapeutic response for a particular patient, composition and mode of administration, without being intolerably toxic to the patient. In certain cases,dosages may deviate from the stated amounts, in particular as a function of age, gender, body weight, diet and general health status of the patient, route of administration, individual response to the active ingredient, nature of the preparation, and time or interval over which administration takes place. Thus, it may be satisfactory in some cases to manage with less than the aforementioned minimum amount, whereas in other cases the stated upper limit may be exceeded. It may in the event of administration of larger amounts be advisable to divide these into multiple individual doses spread over the day.

### Evaluation of the Activity of the Compounds

Standard physiological, pharmacological and biochemical procedures are available for testing the compounds to identify those that possess biological activity as AMPK activators. For example, AMPK activation by test compounds may be measured in a cell based assay where activation of intracellular AMPK in porcine kidney cells (LLC-PK1, Lilly Laboratories) may be measured in terms of phosphorylation of the AMPK substrate, acetyl-coenzyme A carboxylase (ACC), to pACC and also by quantifying phosphorylated AMPK, using Meso Scale Discovery (MSD) assay, ELISA or Western blot as read out (Myers et al., Science, 2017, 357: 507-511). AMPK activation by test compounds may also be evaluated using a ³³P-based assay to measure ³³P phosphorylated ACC or phosphorylation of a synthetic derivative of ACC (SAMS peptide) which involves incubating the test compound with AMPK complex phosphorylated on the activation loop Thr172 of the a-subunit of the enzyme, followed by addition of protein phosphatase 2A catalytic subunit (PP2A) to dephosphorylate pThr172, which is then quenched with okadaic acid, and followed by the addition of substrate and ATP including tracer amounts of ³³P ATP. AMPK activity is measured in terms of substrate ³³P phosphorylation using scintillation counter (WO2013153479). AMPK activators may operate through different pharmacological modes of action, for example, as an allosteric activator of AMPK or through protection of the pThr172 form of the enzyme. Certain assays may determine the mode of action of AMPK activators, such as a time-resolved fluorescence resonance energy transfer (TR-FRET) assay in which test compounds are incubated with recombinantly expressed pThr172 phosphorylated AMPK complex and recombinantly expressed PP2A to measure compounds ability to protect the pThr172 AMPK from desphosphorylation as a first step, followed by quenching of PP2A and addition of ATP and substrate to measure the test compound's to induce AMPK activation. This TR-FRET assay may be used to evaluate compound activity against the various isoforms of AMPK. (See Cameron et al. J. Med. Chem. 2016, 59(17): 8068-8081).

Putative AMPK activators that show good potency and β1-selectivity in the enzymatic assay may be assessed for their in vitro permeability in Caco-2 cell monolayers cultured for 14-18 days on 96-well HTS transwell plate (seeding density: 34,300 cells/transwell). Select compounds of Formula (I) provided herein, diluted to 5 μM concentration in HBSS buffer (10 mM HEPES, pH 7.4) were added to either the apical or basolateral side of the Caco-2 cell monolayers and incubated at 37° C. in a cell culture incubator for 2 h. At the end of incubation, samples were obtained from the contralateral sides and the compound levels were assessed by liquid chromatography—tandem mass spectrometry (LC-MS/MS) assay to determine the apparent permeability co-efficients Pₐₚₚ (A→B), Pₐₚₚ (B→A) and efflux ratio (ER=Pₐₚₚ (B→A)/Pₐₚₚ (A→B)).

Select AMPK activators that show acceptable Caco-2 permeability profile may be assessed for in vitro metabolic stability in cryopreserved hepatocyte suspensions using loss-of-parent approach. Select compounds of Formula (I) provided herein at 1 μM final conc. were incubated with cryopreserved hepatocyte suspensions (rat or human; 0.1×10⁶ cells in 0.2 mL volume) in a 96-well plate at 37° C. for up to 2 h. During the incubation, samples were obtained at different time points and analyzed by LC-MS/MS assay for the parent compound. From the percent parent remaining vs. time curve, half-life, in vitro intrinsic clearance, and scaled-up clearance values were calculated. Key lead compounds were also assessed for metabolic stability in dog and monkey hepatocyte suspensions.

Select AMPK activators of interest may be assessed in vitro for their potential to inhibit the major cytochrome P450 enzymes such as CYP1A2, CYP2B6, CYP2C8, CYP2C9, CYP2C19, CYP2D6, and CYP3A4. Human liver microsomes (150-donor, mixed gender; 0.2 mg/mL) were incubated at 37° C. with respective probe substrates for the individual CYP enzymes in the presence of the select compounds of Formula (I) provided herein at different concentrations (0.001-50 μM) and NADPH (cofactor; 1 mM) for up to 20 min. The reactions were quenched at the designated time points and the metabolite formation was assessed by LC-MS/MS, to determine the IC₅₀ (test compound concentration that produces 50% inhibition of the activity of a CYP enzyme). Select compounds were followed up with IC₅₀ shift assays to determine the potential for time-dependent inhibition of CYP3A4 enzyme and any other CYP enzyme for which reversible inhibition was observed.

Select AMPK compounds with good in vitro ADME properties may then be evaluated for in vivo pharmacokinetics and oral bioavailability in rats. After administration of 2 mg/kg IV and 10 mg/kg PO doses of select compounds of Formula (I) in suitable formulations to rats, plasma samples were obtained at different time points up to 24 h, and compound levels were quantified by LC-MS/MS. Pharmacokinetic parameters (e.g., CLₚ, Vdₛₛ, AUC_{0-24 h}, oral bioavailability) were estimated by non-compartmental analysis (NCA) of the time vs. plasma concentration data. In addition, trough concentrations of select compounds of Formula (I) were measured in rat kidney tissues obtained at 24 h after PO administration to determine the kidney-to-plasma ratio (K_{p, kidney}). Select compounds of Formula (I) evaluated in rat pharmacokinetic studies were also assessed for plasma protein binding and kidney tissue binding by using ultracentrifugation approach. Fraction unbound (Fu) values in rat plasma and rat kidney homogenate were then used to determine the unbound concentrations achieved in rat plasma and kidney, and also to calculate the unbound kidney-to-unbound plasma ratio (K_{p,uu,kidney}) Pharmacokinetic properties of key lead compounds of interest were also evaluated in higher species such as beagle dogs and cynomolgus monkeys to guide human PK prediction by allometric scaling.

In certain embodiments, compounds of Formula (I) consistently showed a higher unbound kidney-to-unbound plasma ratio (i.e., K_{p,uu,kidney}»1) in rats, renal uptake transporters were believed to play a role in the renal uptake of these compounds. In certain embodiments, based on the chemical class of the compounds of Formula (I) disclosed herein and the known substrate profiles of various renal uptake transporters, organic anion transporters 1 and 3 (OAT1 and 3) from the SLC transporter family are postulated to be the likely transporters involved in the renal disposition of these compounds. Certain compounds of Formula I were therefore assessed for the substrate potential for rat Oat1, human OAT1, and human OAT3 using cell lines stably over-expressing these transporter proteins.

The in vivo pharmacodynamic (PD) effect of AMPK activators in the kidney can be determined by measuring phosphorylated AMPK relative to total AMPK. More specifically, Sprague Dawley rats are dosed with a compound of Formula (I) or a vehicle control by oral gavage and sacrificed at designated time points up to 24 hours later. Kidney tissue is rapidly harvested, and a transverse slice is flash frozen in liquid nitrogen. Samples are then processed on a pre-chilled BioPulverizer (BioSpec, 59012MS) and then homogenized in cold lysis buffer with protease and phosphatase inhibitors (Thermo Fisher, 78446) using a probe sonicator. Protein concentrations are quantified by BCA assay (Thermo Fisher, 23225) and phospho-AMPK and total AMPK are measured by ELISA (Cell Signaling Technologies, 7959C and 7961C) as recommended by supplier.

### Methods of Use

Provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via AMPK activity. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via the β1-containing isoform of AMPK. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via AMPK α1/β1/γ1 or α1/β2/γ1 isoform. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via activation of AMPK. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via activation of the β1-containing isoform of AMPK. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via activation of the AMPK α1/β1/γ1 or α1/β2/γ1 isoform. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by or otherwise affected via AMPK α1/β1/γ1 isoform. In yet certain embodiments, provided herein are methods of using the compounds disclosed herein, or pharmaceutically acceptable salts, solvates, hydrates thereof, or pharmaceutical compositions thereof, for the treatment, prevention or amelioration of a disease or disorder that is mediated by the activation of the AMPK α1/β1/γ1 isoform.

In certain embodiments, provided herein are methods of treating chronic kidney disease (CKD), end stage renal disease (ESRD), diabetic nephropathy, acute kidney injury, polycystic liver disease or polycystic kidney disease (PKD), also referred to as polycystic renal disease. In certain embodiments, the polycystic kidney disease is autosomal dominant PKD (ADPKD). In certain embodiments, the polycystic kidney disease is autosomal dominant PKD type 1. In certain embodiments, the polycystic kidney disease is autosomal dominant PKD type 2. In certain embodiments, the polycystic kidney disease is associated with at least one mutation in the PKD1 gene or the PKD2 gene. In certain embodiments, the polycystic kidney disease is autosomal recessive PKD (ARPKD). In certain embodiments, the polycystic kidney disease is associated with at least one mutation in the PKHD1 gene. In certain embodiments, the polycystic kidney disease is associated with at least one mutation in the DZIP1L gene.

In certain embodiments, provided herein are methods of treating obesity, metabolic syndrome, diabetes, inflammation or cancer. In yet certain embodiments, provided herein are methods of treating obesity, Type II diabetes or dyslipidemia.

### Combination Therapy

It will be understood by those skilled in the art that the compounds, isomers, and pharmaceutically acceptable salts, solvates and hydrates provided herein, and pharmaceutical compositions and formulations comprising such compounds, isomers, and pharmaceutically acceptable salts, solvates and hydrates thereof can be used in a variety of combination therapies to treat the diseases, disorders and conditions described above. Thus, also contemplated herein is the use of compounds, isomers, pharmaceutically acceptable salts, solvates and hydrates thereof, and pharmaceutical compositions comprising such compounds, isomers, and pharmaceutically acceptable salts, solvates and hydrates thereof in combination with other active pharmaceutical agents for the treatment of the diseases, disorders and conditions described herein.

In one embodiments, such additional pharmaceutical agents include without limitation, vasopressin V2 receptor antagonists, glucosylceramide synthase inhibitors, nuclear factor erythroid 2-related factor 2 (NRF2) activators, cystic fibrosis transmembrane conductance regulatory (CFTR) inhibitors, angiotensin converting enzyme (ACE) inhibitors, angiotensin II receptor antagonists, sodium glucose co-transporter-2 (SGLT2) inhibitors or EGFR inhibitors. In yet certain embodiments, the additional pharmaceutical agent is a vasopressin V2 receptor antagonist.

The compound or pharmaceutically acceptable salts, solvates and hydrates thereof, or the pharmaceutical composition thereof, may be administered simultaneously with, prior to, after or during the administration of one or more of the above agents. Pharmaceutical compositions containing a compound provided herein, or a pharmaceutically acceptable sale, solvate or hydrate thereof, and one or more of the above additional agents are also provided.

In one embodiment, provided herein is a combination therapy that treats or prevents polycystic kidney disease, said therapy comprising the administration to a subject in need thereof, one of the compounds or pharmaceutically acceptable salts, solvates and hydrates thereof, or the pharmaceutical composition thereof, with one or more vasopressin V2 receptor antagonist. In certain embodiments, the vasopressin V2 receptor antagonist is tolvaptan.

### EXAMPLES

The starting materials used for the synthesis were prepared according to known literature procedures or obtained from commercial sources, such as, but not limited to, Sigma-Aldrich, Fluka, Acros Organics, Alfa Aesar, VWR Scientific, and the like. Nuclear Magnetic Resonance (NMR) analysis was conducted using a 400 MHz spectrometer with an appropriate deuterated solvent. Chemical shifts (δ) are expressed in units of parts per million (ppm). LCMS analysis was conducted using a Shimadzu LCMS-2020 with a Ascentis Express C18 2.7 μM, 3.0×50 mm column, eluting with 95:5 to 0:100 H₂O: CH₃CN+0.05% trifluoroacetic acid at a flow rate of 1.5 mL/min over 3.0 minutes. Alternatively, LCMS analysis was conducted using a a Waters Acquity UPLC with a QDA MS detector using a Waters C18 BEH 1.7 μm, 2.1×50 mm column, eluting with 95:5 to 0:100 H₂O:MeCN+0.1% formic acid at a flow rate of 0.6 mL/min over 3.5 minutes. The QDA MS detector was set up to scan under both positive and negative mode ions ranging from 100-1200 Daltons. General methods for the preparation of compounds can be modified using appropriate reagents and conditions for the introduction of the various moieties found in the structures as provided herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Standard abbreviations and acronyms as defined in Journal of Organic Chemistry' s Author's Guideline at http://pubs.acs.org/userimages/ContentEditor/1218717864819/joceab_abbreviations.pdf are used herein. Other abbreviations and acronyms used herein are as follows:
BSA bovine serum albumin
EGTA (ethylene glycol-bis(β-aminoethyl ether)-N,N,N′,N′-tetraacetic acid
EtOAc ethyl acetate
EtOH ethanol
HEPES (4-(2-hydroxyethyl)-1-piperazine ethanesulfonic acid
HPLC high pressure liquid chromatography
LCMS liquid chromatography—mass spectrometry
LiHMDS lithium bis(trimethylsilyl)amide
MeOH methanol
MeONa sodium methoxide
mg milligrams
mL milliliter
mmol millimoles
Pd(dppf)Cl₂ [1,1′-bis(diphenylphosphino)ferrocene]dichloropalladium(II)
Pd(dtbpf)Cl₂ [1,1′-bis(di-tert-butylphosphino)ferrocene]dichloropalladium(II)
Pd(PPh₃)₄ tetrakis(triphenylphosphine)palladium(0)
PEPPS [1,3-Bis(2,6-Diisopropylphenyl)imidazol-2-ylidene](3-chloropyridyl)palladium(II) dichloride
Pd-PEPPSI-IPent^{Cl} dichloro[1,3-bis(2,6-di-3-pentylphenyl)imidazol-2-ylidene](3-chloropyridyl)palladium(II)
PPh₃ triphenylphosphine
RuPhos 2-dicyclohexylphosphino-2′,6′-diisopropoxybiphenyl
RuPhos-Pd-G3 (2-dicyclohexylphosphino-2′,6-diisopropoxy-1,1′-biphenyl)[2-(2′-amino-1,1′-biphenyl)]palladium(II) methanesulfonate
sat. saturated
TMSOTf trimethylsilyl trifluoromethanesulfonate
μL microliter
μm micrometers
UHPLC ultra high performance liquid chromatography
μW microwave reactor

In an illustrative method, the pyridinyl indole compounds of Formula (I) may be prepared according to the synthetic routes outlined in Scheme 1. Various spiro-, bridged- or fused-bicyclic amines (A-2) can react with halogenated-pyridine intermediate A-1 (e.g. 3-bromo-6-chloro-2-methoxypyridine, CAS: 1211526-62-3) when treated with a base such as potassium carbonate at an elevated temperature (e.g. 110° C.), yielding the desired pyridine intermediate A-3 (e.g. Intermediate C). Intermediate A-3 can undergo Suzuki-Miyaura cross-coupling with boronate intermediate such as A-4 (e.g. methyl 6-chloro-5-(5,5-dimethyl-1,3,2-dioxaborinan-2-yl)-1H-indole-3-carboxylate, CAS: 1467060-68-9) under Suzuki-Miyaura cross -coupling conditions (for example, 10 mol % Pd(dppf)Cl₂, K₃PO₄ and dioxane at 90° C.), resulting in the 5-pyridyl indole of Formula (I). Upon saponification/acidification, the corresponding indole 3-carboxylic acid can be accessed.

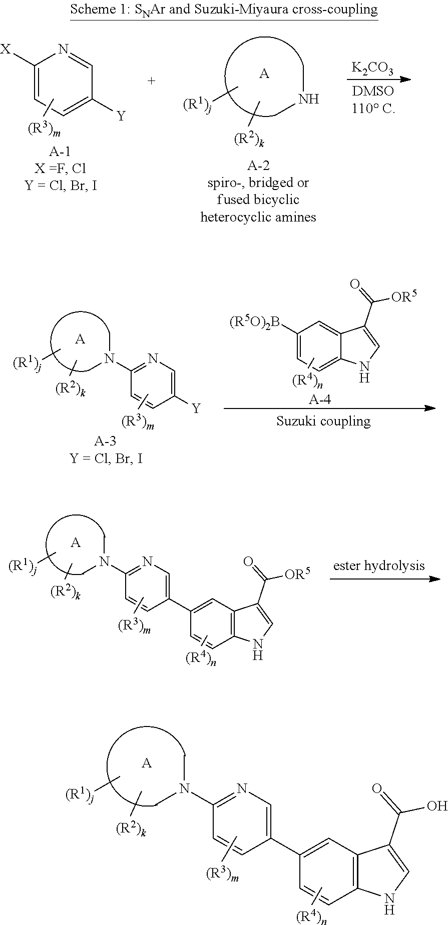

In an illustrative method, the pyridinyl indole compounds of Formula (I) may also be prepared according to the synthetic routes outlined in Scheme 2. Using a Pd catalyzed aryl amination of the advanced intermediate B-1, such as Intermediate A or B, the aminopyridyl indole carboxylate of Formula (I) can be accessed directly. Depending on the secondary amine B-2, either RuPhos-Pd-G2 or Pd-PEPPSI-IPent^{Cl} can be used to facilitate the desired C-N bond formation. Upon saponification/acidification, the corresponding indole 3-carboxylic acid can be accessed.

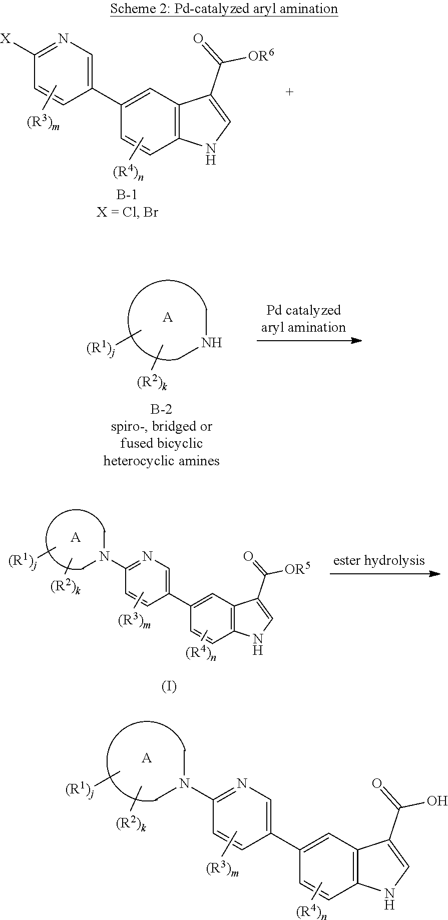

In an illustrative method, the pyridinyl indole compounds of Formula (I) may also be prepared according to the synthetic routes outlined in Scheme 3. Some secondary amines can also undergo a late-stage S_{N}Ar reaction with the advanced pyridyl intermediate C-2, such as Intermediate A or B below, providing quick access to compounds of Formula (I) such as the aminopyridyl indole carboxylate C-3. Upon saponification/acidification, the corresponding indole 3-carboxylic acid can be accessed.

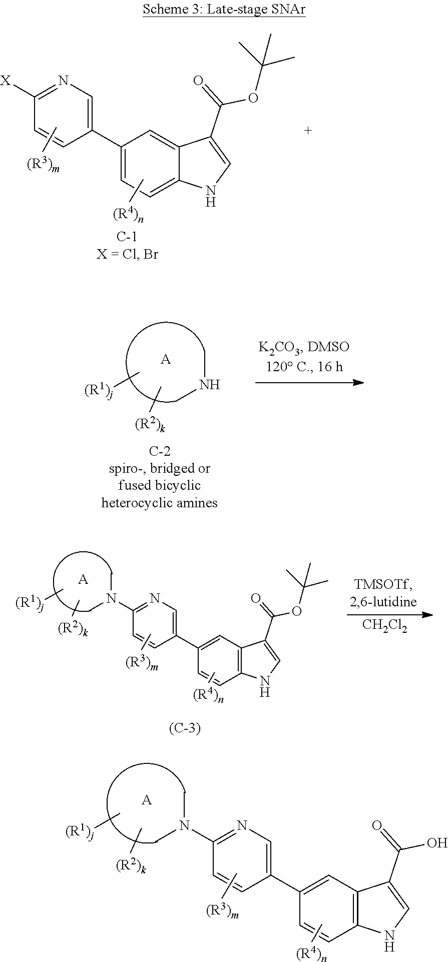

### PREPARATION OF INTERMEDIATES

### Preparation of Intermediate A: Tert-Butyl 5-(6-bromo-2-methoxypyridin-3-yl)-6-chloro-1H-indole-3-carboxylate

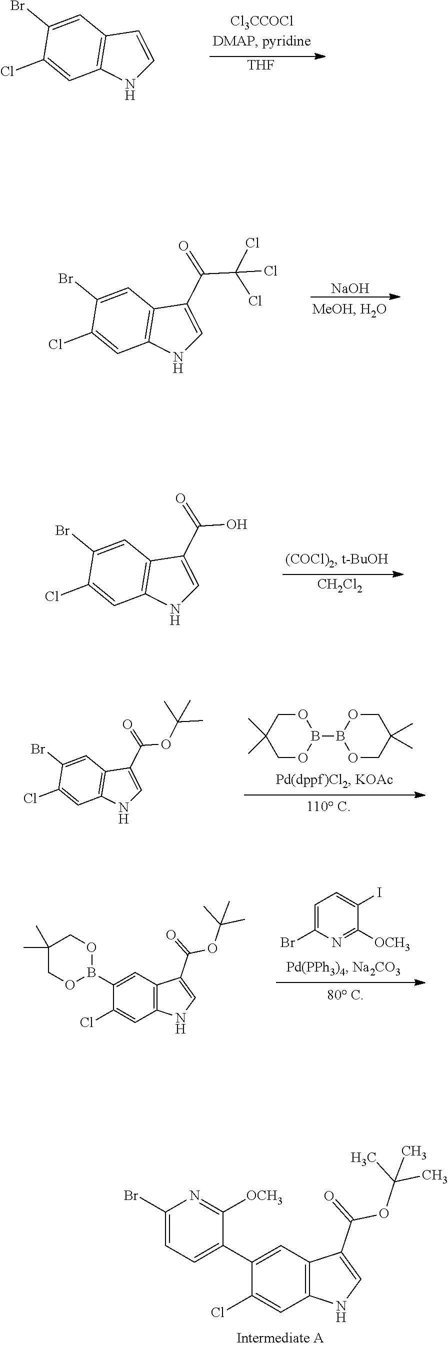

Step 1: Preparation of 1-(5-bromo-6-chloro-1H-indol-3-yl)-2,2,2-trichloroethanone: Into a round-bottom flask was added 5-bromo-6-chloro-1H-indole (1.0 equiv), THF (0.2 M), DMAP (0.1 equiv) and pyridine (2.6 equiv). This was followed by addition of trichloroacetyl chloride (2.4 equiv) dropwise with stirring at 0° C. The resulting solution was stirred for 48 hours at 60° C. The reaction was then quenched with water. The resulting solution was extracted three times with ethyl acetate and the organic layers were combined and concentrated. The resulting residue was applied onto a silica gel column and purified with column chromatography eluting with 60:40 EtOAc:petroleum ether. The title product was obtained as a light yellow solid (82% yield).

Step 2: Preparation of 5-bromo-6-chloro-1H-indole-3-carboxylic acid: Into a round-bottom flask was added 1-(5-bromo-6-chloro-1H-indol-3-yl)-2,2,2-trichloroethanone (1.0 equiv), CH₃OH (0.5 M), water (0.4 M) and NaOH (2.5 equiv). The resulting solution was stirred for 16 hours at 60° C. The resulting solution was extracted three times with ethyl acetate. The aqueous layers were combined and the pH value of the aqueous solution was adjusted to pH ˜6 with aqueous HCl (6 M). The resulting solution was extracted three times with ethyl acetate. The organic layers were combined, dried over anhydrous sodium sulfate, filtered, and concentrated to afford the title product as a light brown solid (69% yield).

Step 3: Preparation of tert-butyl 5-bromo-6-chloro-1H-indole-3-carboxylate: Into a round-bottom flask was added 5-bromo-6-chloro-1H-indole-3-carboxylic acid (1.0 equiv), CH₂Cl₂ (0.07 M), (CO)₂Cl₂ (3 equiv) and DMF (0.1 equiv). The resulting solution was stirred for 3 h at 30° C. The resulting mixture was concentrated under reduced pressure. The t-BuOH (0.07 M) and t-BuOK (2 equiv) reagents were added and the resulting solution was stirred for another 2 hours at 30° C. The reaction was quenched aqueous saturated NH₄Cl solution. The resulting mixture was extracted three times with ethyl acetate and the organic layers were combined and concentrated. The residue was applied onto a silica gel column and purified by column chromatography eluting with 30:70 EtOAc:petroleum ether. The title product was obtained as a light yellow solid (55% yield).

Step 4: Preparation of tert-butyl 6-chloro-5-(5,5-dimethyl-1,3,2-dioxaborinan-2-yl)-1H-indole-3-carboxylate: Into a round-bottom flask purged and maintained with an inert atmosphere of nitrogen was added tert-butyl 5-bromo-6-chloro-1H-indole-3-carboxylate (1.0 equiv), 2-(5,5-dimethyl-1,3,2-dioxaborinan-2-yl)-5,5-dimethyl-1,3,2-dioxaborinane (1.5 equiv), Pd(dppf)Cl₂ (0.1 equiv), KOAc (2 equiv) and dioxane (0.09 M). The resulting suspension was heated for 2 hours at 110° C. The resulting mixture was diluted with ethyl acetate and the solids were removed by filtration. The resulting filtrate was concentrated. The residue obtained from the filtrate was applied onto a silica gel column and purified by column chromatography eluting with 45:55 EtOAc:petroleum ether to afford the title product as a light yellow solid (26% yield).

Step 5: Preparation of tert-butyl 5-(6-bromo-2-methoxypyridin-3-yl)-6-chloro-1H-indole-3-carboxylate: Into a 3-necked, round-bottom flask purged and maintained with an inert atmosphere of nitrogen was added tert-butyl 6-chloro-5-(5,5-dimethyl-1,3,2-dioxaborinan-2-yl)-1H-indole-3-carb oxylate (1.0 equiv), 6-bromo-3-iodo-2-methoxypyridine (1.1 equiv), Pd(PPh₃)₄ (0.1 equiv), Na₂CO₃ (3 equiv), THF (0.3 M) and H₂O (0.05 M). The resulting mixture was heated for 16 hours at 80° C. The reaction was then quenched with water and the resulting biphasic mixture was extracted three times with ethyl acetate. The organic layers were combined and concentrated. The crude product was purified by column chromatography through a C18 column, eluting with 80:20 to 35:65 water:CH₃CN as a gradient over 30 min. The title product was obtained as a light yellow solid (58% yield).

### Preparation of Intermediate B: Methyl 5-(6-bromo-2-methoxypyridin-3-yl)-6-chloro-1H-indole-3-carboxylate

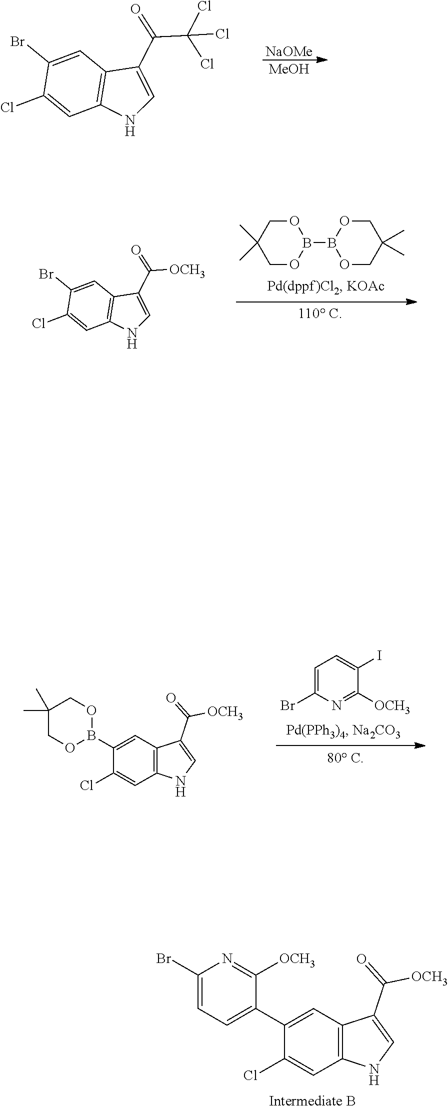

Step 1: Preparation of methyl 5-bromo-6-chloro-1H-indole-3-carboxylate: Into a round-bottom flask was added 1-(5-bromo-6-chloro-1H-indol-3-yl)-2,2,2-trichloroethanone (1.0 equiv) and MeOH (0.04 M). This was followed by the addition of NaOMe in MeOH (1.1 equiv) dropwise with stirring at 0° C. The resulting mixture was heated for 16 hours at 45° C. The reaction was then quenched by water and the resulting solution was extracted three times with ethyl acetate. The organic layers were combined and concentrated. The residue from the organic layers was applied onto a silica gel column and purified by column chromatography eluting with 60:40 EtOAc:petroleum ether to afford the title product as a yellow solid (68% yield).

Step 2: Preparation of methyl 6-chloro-5-(5,5-dimethyl-1,3,2-dioxaborinan-2-yl)-1H-indole-3-carboxylate: Into a round-bottom flask, purged and maintained with an inert atmosphere of nitrogen, was added methyl 5-bromo-6-chloro-1H-indole-3-carboxylate (1.0 equiv), 2-(5,5-dimethyl-1,3,2-dioxaborinan-2-yl)-5,5-dimethyl-1,3,2-dioxaborinane (1.2 equiv), Pd(dppf)Cl₂ (0.1 equiv), KOAc (2.0 equiv) and dioxane (0.04 M). The resulting mixture was heated for 2 hours at 110° C. The resulting mixture was diluted with ethyl acetate and filtered and the resulting filtrate was concentrated. The residue was applied onto a silica gel column and purified by column chromatography eluting with 30:70 EtOAc:petroleum ether to afford the title product as a white solid (49% yield).

Step 3: Preparation of methyl 5-(6-bromo-2-methoxypyridin-3-yl)-6-chloro-1H-indole-3-carboxylate: Into a 3-necked round-bottom flask, purged and maintained with an inert atmosphere of nitrogen, was added methyl 6-chloro-5-(5,5-dimethyl-1,3,2-dioxaborinan-2-yl)-1H-indole-3-carboxylat (1.0 equiv), 6-bromo-3-iodo-2-methoxypyridine (1.2 equiv), Pd(PPh₃)₄ (0.1 equiv), Na₂CO₃ (3.0 equiv), dioxane (0.18 M) and H₂O (0.04 M). The resulting mixture was heated for 16 hours at 80° C. The reaction was cooled and quenched by water. The solids were filtered and the resulting filtrate was extracted three times with ethyl acetate. The organic layers were combined and concentrated under vacuum. The crude product was purified by column chromatography through a C18 column, eluting with 100:0 to 48:52 water:CH₃CN as a gradient. The title product was obtained as a yellow solid (34% yield).

### Preparation of Intermediate C: 6-fluoro-5-(2-methoxy-6-[2-oxa-6-azasniro[3.3]hentan-6-yl]pyridin-3-yl)-1H-indole-3-carboxylic acid

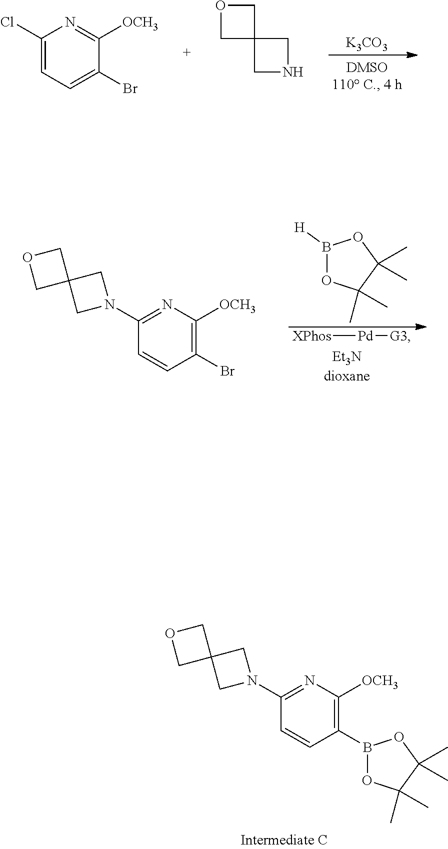

Step 1: Preparation of 6-(5-bromo-6-methoxypyridin-2-yl)-2-oxa-6-azaspiro[3.3]heptane: To a degassed solution of 3-bromo-6-chloro-2-methoxy-pyridine (1.0 equiv) and 2-oxa-6-azaspiro[3.3]heptane (1.0 equiv) in DMSO (0.3 M) was added potassium carbonate (3.0 equiv). The resulting suspension was heated to 110° C. for 4 hours. LCMS indicated formation of product. This reaction mixture was cooled to 24° C., loaded onto a silica gel pre-cartridge and dried under vacuum. The mixture was purified by column chromatography through silica gel eluting with 100:0 to 40:60 hexanes:EtOAc as a gradient. The desired product containing fractions were concentrated and dried under vacuum to afford a clear oil (50% yield).

Step 2: Preparation of 6-fluoro-5-(2-methoxy-6-[2-oxa-6-azaspiro[3.3]heptan-6-yl]pyridin-3-yl)-1H-indole-3-carboxylic acid: To a stirred mixture of 6-(5-bromo-6-methoxypyridin-2-yl)-2-oxa-6-azaspiro[3.3]heptane (1.0 equiv), XPhos-Pd-G3 (0.1 equiv) and Et₃N (3.0 equiv) in dioxane (0.5 M) was added 4,4,5,5-tetramethyl-1,3,2-dioxaborolane (4 equiv) dropwise at 0° C. under a nitrogen atmosphere. The resulting mixture was warmed to 24° C. and heated for 15 minutes at 100° C. under nitrogen atmosphere. LC-MS analysis indicated consumption of starting material and formation of product. The residue was dissolved in EtOAc and the resulting mixture was filtered. The filter cake was washed with EtOAc and the filtrate was concentrated under reduced pressure. The residue was purified by reverse phase flash chromatography through a C18 column, eluting with 50:50 to 60:40 CH₃CN:water as a gradient. The resulting mixture was concentrated under vacuum to afford the title product as a white solid (23% yield).

### PREPARATION OF EXAMPLES

### Example 1

### Preparation of 6-chloro-5-(2-methoxy-6-(2-oxa-6-azaspiro[3.3]heptan-6-yl)pyridin-3-yl)-1H-indole-3-carboxylic Acid

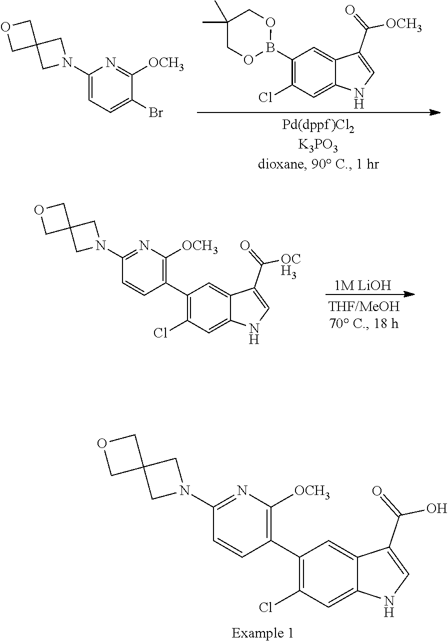

Step 1: Preparation of methyl 6-chloro-5-(2-methoxy-6-(2-oxa-6-azaspiro[3.3]heptan-6-yl)pyridin-3-yl)-1H-indole-3-carboxylate: Into a reaction flask was added 6-(5-bromo-6-methoxy-2-pyridyl)-2-oxa-6-azaspiro[3.3]heptane (1.0 equiv), methyl 6-chloro-5-(5,5-dimethyl-1,3,2-dioxaborinan-2-yl)-1H-indole-3-carboxylate (1.0 equiv), potassium phosphate aqueous solution (3 M, 3.0 equiv), Pd(dppf)Cl₂ (10 mol %) and dioxane (0.4 M). The resulting biphasic mixture was degassed with nitrogen for 10 min. The mixture was heated to 90° C. for 30 minutes after which LCMS indicated completion of reaction. This reaction mixture was cooled to 24° C., loaded onto a 20 g silica gel pre-cartridge and dried under vacuum. The mixture was purified by column chromatography through silica gel, eluting with 95:5 to 0:100 hexanes:EtOAc as a gradient. The desired product containing fractions were concentrated and dried under vacuum to afford a white solid (56% yield).

Step 2: Preparation of 6-chloro-5-(2-methoxy-6-(2-oxa-6-azaspiro[3.3]heptan-6-yl)pyridin-3-yl)-1H-indole-3-carboxylic acid: Into a rection flask was added methyl 6-chloro-5-(2-methoxy-6-(2-oxa-6-azaspiro[3.3]heptan-6-yl)pyridin-3-yl)-1H-indole-3-carboxylate (1.0 equiv) and 1 M aqueous LiOH/THF/MeOH (3 equiv LiOH, v/v/v, 1/1/1). The resulting solution was heated to 70° C. for 18 hours. LCMS indicated completion of hydrolysis, after which the mixture was cooled and the reaction quenched with formic acid to pH ˜2. The mixture was loaded onto a reverse phase pre-cartridge. Purification by a reverse phase column chromatography through a C18 column, eluting with 90:10 to 0:100 water:CH₃CN+0.1% formic acid as a gradient. The desired product containing fractions were concentrated and lyophilized overnight to yield the title product as a white powder (34% yield). ¹H-NMR (400 MHz, d₆-DMSO): δ 11.87 (s, 1H), 8.01 (s, 1H), 7.79 (s, 1H), 7.53 (s, 1H), 7.31 (d, J=7.8 Hz, 1H), 5.98 (d, J=7.9 Hz, 1H), 4.72 (s, 4H), 4.15 (s, 4H), 3.72 (d, J=1.6 Hz, 3H). LC-MS (ESI) m/z 400 (M+H)⁺.

The following compounds were prepared in a similar manner as Example 1, by replacing 2-oxa-6-azaspiro[3.3]heptane with the corresponding secondary amines in the first step.

| Example | Structure | MW | MS (ESI+) |
|---|---|---|---|
| Example 2 | 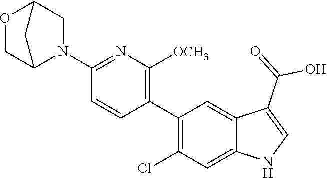 [SMILES: COc1nc(N2CC3CC2CO3)ccc1-c1cc2c(C(=O)O)c[nH]c2cc1Cl] rac-5-(6-(2-oxa-5-azabicyclo[2.2.1]heptan-5-yl)-2- methoxypyridin-3-yl)-6-chloro-1H-indole-3-carboxylic acid | 399.83 | 400 (M + 1)⁺ |
| Example 3 | 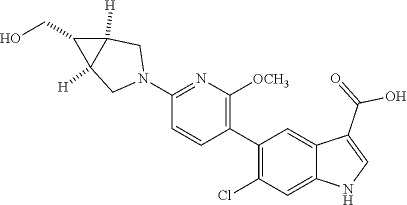 [SMILES: COc1nc(N2C[C@@H]3[C@@H](CO)[C@@H]3C2)ccc1-c1cc2c(C(=O)O)c[nH]c2cc1Cl] 6-chloro-5-(6-((trans)-6-(hydroxymethyl)-3-azabicyclo [3.1.0]hexan-3-yl)-2-methoxypyridin-3-yl)-1H-indole- 3-carboxylic acid | 413.86 | 414 (M + 1)⁺ |
| Example 4 | 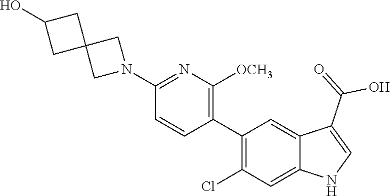 [SMILES: COc1nc(N2CC3(CC(O)C3)C2)ccc1-c1cc2c(C(=O)O)c[nH]c2cc1Cl] 6-chloro-5-(6-(6-hydroxy-2-azaspiro[3.3]heptan-2-yl)- 2-methoxypyridin-3-yl)-1H-indole-3-carboxylic acid | 413.86 | 414 (M + 1)⁺ |
| Example 5 | 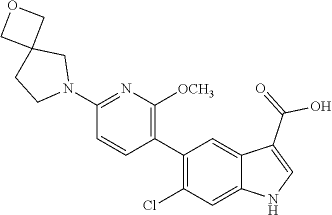 [SMILES: COc1nc(N2CCC3(COC3)C2)ccc1-c1cc2c(C(=O)O)c[nH]c2cc1Cl] 6-chloro-5-(2-methoxy-6-(2-oxa-6-azaspiro[3.4]octan- 6-yl)pyridin-3-yl)-1H-indole-3-carboxylic acid | 413.86 | 414 (M + 1)⁺ |
| Example 6 | 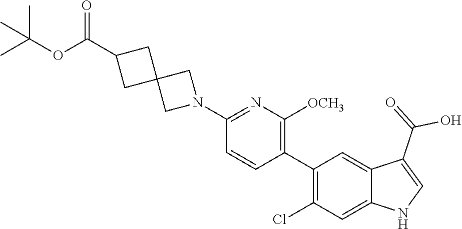 [SMILES: COc1nc(N2CC3(CC(C(=O)OC(C)(C)C)C3)C2)ccc1-c1cc2c(C(=O)O)c[nH]c2cc1Cl] 5-(6-(6-(tert-butoxycarbonyl)-2,6-diazaspiro[3.3]heptan- 2-yl)-2-methoxypyridin-3-yl)-6-chloro-1H-indole- 3-carboxylic acid | 498.96 | 499 (M + 1)⁺ |
| Example 7 | 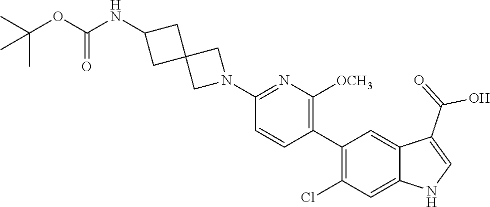 [SMILES: COc1nc(N2CC3(CC(NC(=O)OC(C)(C)C)C3)C2)ccc1-c1cc2c(C(=O)O)c[nH]c2cc1Cl] 5-(6-(6-((tert-butoxycarbonyl)amino)-2-azaspiro[3.3] heptan-2-yl)-2-methoxypyridin-3-yl)-6-chloro-1H- indole-3-carboxylic acid | 512.99 | 513 (M + 1)⁺ |
| Example 8 | 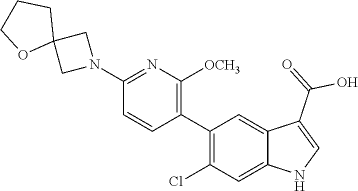 [SMILES: COc1nc(N2CC3(CCCO3)C2)ccc1-c1cc2c(C(=O)O)c[nH]c2cc1Cl] 6-chloro-5-(2-methoxy-6-(5-oxa-2-azaspiro[3.4]octan- 2-yl)pyridin-3-yl)-1H-indole-3-carboxylic acid | 413.86 | 414 (M + 1)⁺ |
| Example 9 | 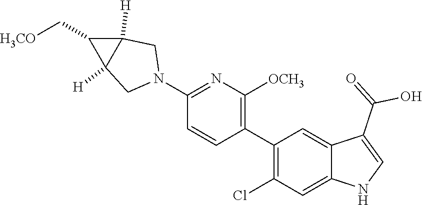 [SMILES: COC[C@@H]1[C@H]2CN(c3ccc(-c4cc5c(C(=O)O)c[nH]c5cc4Cl)c(OC)n3)C[C@@H]12] 6-chloro-5-(2-methoxy-6-((trans)-6-(methoxymethyl)- 3-azabicyclo[3.1.0]hexan-3-yl)pyridin-3-yl)-1H-indole- 3-carboxylic acid | 427.89 | 428 (M + 1)⁺ |

### Example 10

### Preparation of 5-(6-(6-amino-2-azaspiro[3.3]heptan-2-yl)-2-methoxypyridin-3-yl)-6-chloro-1H-indole-3-carboxylic Acid

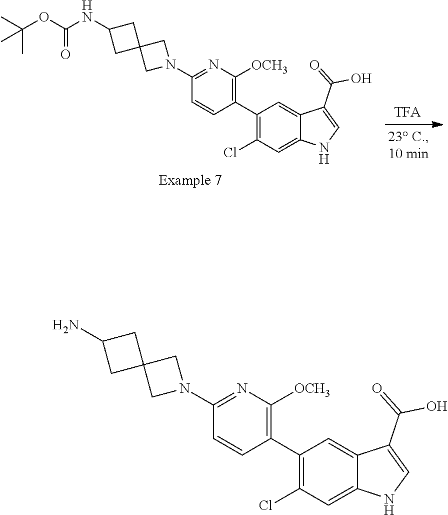 [SMILES: COc1nc(N2CC3(CC(NC(=O)OC(C)(C)C)C3)C2)ccc1-c1cc2c(C(=O)O)c[nH]c2cc1Cl ; COc1nc(N2CC3(CC(N)C3)C2)ccc1-c1cc2c(C(=O)O)c[nH]c2cc1Cl]

To a reaction flask was added Example 7 and trifluoroacetic acid (10 equiv). The solution was stirred at 24° C. for 10 minutes. LCMS analysis indicated completion of the reaction. Purification by reverse phase column chromatography through a C18 column, eluting with 70:30 to 0:100 water:CH₃CN+0.1% formic acid as a gradient. The desired product containing fractions were combined and lyophilized overnight to yield the title product as a white solid (32% yield). LC-MS (ESI) m/z 413 (M+H)⁺.

The following compound was prepared in a similar manner as Example 10, by replacing Example 7 with Example 6.

| Example | Structure | MW | MS (ESI+) |
|---|---|---|---|
| Example 11 | 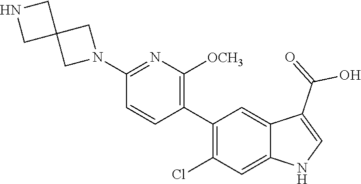 [SMILES: COc1nc(N2CC3(CNC3)C2)ccc1-c1cc2c(C(=O)O)c[nH]c2cc1Cl] 6-chloro-5-(2-methoxy-6-(2,6-diazaspiro[3.3]heptan- 2-yl)pyridin-3-yl)-1H-indole-3 -carboxylic acid | 398.85 | 399 (M + 1)⁺ |

### Example 12

### Preparation of 5-[6-(5-azaspiro[2.3]hexan-5-yl)-2-methoxy-3-pyridyl]-6-chloro-1H-indole-3-carboxylic acid

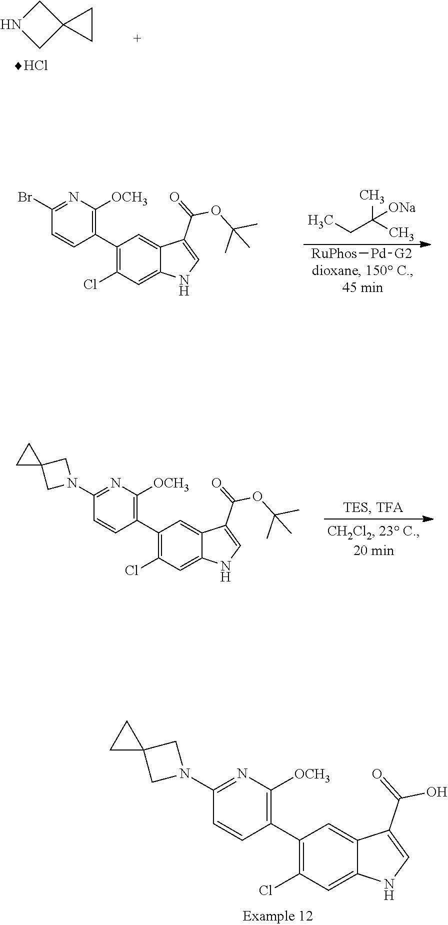

Step 1: Preparation of tert-butyl 5-[6-(5-azaspiro[2.3]hexan-5-yl)-2-methoxy-3-pyridyl]-6-chloro-1H-indole-3-carboxylate: To a glass microwave vial was added Intermediate A (1.0 equiv), RuPhos-Pd-G2 (0.04 equiv), sodium tert-pentoxide (6.0 equiv), and 5-azaspiro[2.3]hexane hydrochloride (2.0 equiv). The solids were suspended in dioxane (0.3 M) and this mixture was degassed before being heated in a microwave reactor to 150° C. for 45 minutes. LCMS analysis indicated completion of reaction. The reaction mixture was cooled to 24° C., loaded onto a 20 g silica gel pre-cartridge, and dried under vacuum. The mixture was purified by column chromatography through silica gel, eluting with 90:10 to 0:100 hexanes:EtOAc as a gradient. The product containing fractions were combined, concentrated, and dried under a reduced pressure to afford a solid (60% yield).

Step 2: Preparation of 5-[6-(5-azaspiro[2.3]hexan-5-yl)-2-methoxy-3-pyridyl]-6-chloro-1H-indole-3-carboxylic acid: To a reaction flask was added tert-butyl 5-[6-(5-azaspiro[2.3]hexan-5-yl)-2-methoxy-3-pyridyl]-6-chloro-1H-indole-3-carboxylate (1.0 equiv), trifluoroacetic acid (50 equiv), triethylsilane (1.5 equiv) and CH₂Cl₂ (0.5 M). The solution was stirred at 24° C. for 20 minutes. LCMS analysis indicated completion of the reaction. The reaction mixture was concentrated, and the resulting residue was loaded directly onto a reverse phase C18 pre-cartridge. Purification by reverse phase column chromatography through a C18 column, eluting with 70:30 to 0:100 water:CH₃CN+0.1% formic acid as a gradient. Fractions containing the desired product were combined and lyophilized overnight to yield the title product as a white solid (50% yield). ¹H-NMR (400 MHZ, d₆-DMSO): δ 11.87 (s, 1H), 8.01 (s, 1H), 7.82 (d, J=1.8 Hz, 1H), 7.54 (d, J=1.8 Hz, 1H), 7.32 (dd, J=7.9, 1.9 Hz, 1H), 5.98 (dd, J=7.9, 1.8 Hz, 1H), 4.04 (s, 4H), 3.72 (s, 3H), 0.66 (s, 4H). LC-MS (ESI) m/z 384 (M+H)⁺.

The following compounds were prepared in a similar manner as Example 12, by replacing 5-azaspiro[2.3]hexane hydrochloride with the corresponding secondary amines in the first step. In some cases, the sodium tert-pentoxide base in the first step may be replaced with LiHDMS (1.0 M in THF).

| Example | Structure | MW | MS (ESI+) |
|---|---|---|---|
| Example 13 | 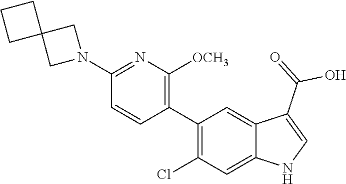 [SMILES: COc1nc(N2CC3(CCC3)C2)ccc1-c1cc2c(C(=O)O)c[nH]c2cc1Cl] 6-chloro-5-(2-methoxy-6-(2-azaspiro[3.3]heptan-2-yl) pyridin-3-yl)-1H-indole-3-carboxylic acid | 397.86 | 398 (M + 1)⁺ |
| Example 14 | 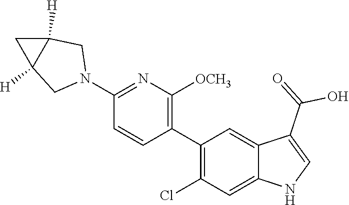 [SMILES: COc1nc(N2C[C@H]3C[C@H]3C2)ccc1-c1cc2c(C(=O)O)c[nH]c2cc1Cl] 5-(6-(3-azabicyclo[3.1.0]hexan-3-yl)-2-methoxypyridin- 3-yl)-6-chloro-1H-indole-3-carboxylic acid | 383.83 | 384 (M + 1)⁺ |
| Example 15 | 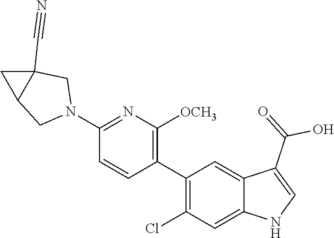 [SMILES: COc1nc(N2CC3CC3(C#N)C2)ccc1-c1cc2c(C(=O)O)c[nH]c2cc1Cl] racemic-6-chloro-5-(6-(1-cyano-3-azabicyclo[3.1.0] hexan-3-yl)-2-methoxypyridin-3-yl)-1H-indole-3- carboxylic acid | 408.84 | 409 (M + 1)⁺ |
| Example 16 | 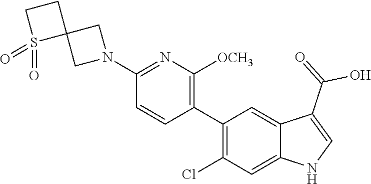 [SMILES: COc1nc(N2CC3(CCS3(=O)=O)C2)ccc1-c1cc2c(C(=O)O)c[nH]c2cc1Cl] 6-chloro-5-(6-(1,1-dioxido-1-thia-6-azaspiro[3.3 ]heptan- 6-yl)-2-methoxypyridin-3-yl)-1H-indole-3-carboxylic acid | 447.89 | 448 (M + 1)⁺ |
| Example 17 | 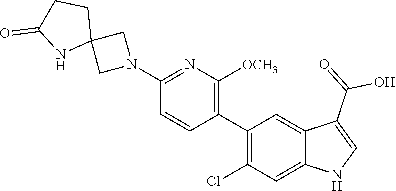 [SMILES: COc1nc(N2CC3(CCC(=O)N3)C2)ccc1-c1cc2c(C(=O)O)c[nH]c2cc1Cl] 6-chloro-5-(2-methoxy-6-(6-oxo-2,5-diazaspiro[3.4] octan-2-yl)pyridin-3-yl)-1H-indole-3-carboxylic acid | 426.86 | 427 (M + 1)⁺ |
| Example 18 | 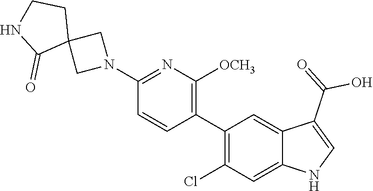 [SMILES: COc1nc(N2CC3(CCNC3=O)C2)ccc1-c1cc2c(C(=O)O)c[nH]c2cc1Cl] 6-chloro-5-(2-methoxy-6-(5-oxo-2,6-diazaspiro[3.4] octan-2-yl)pyridin-3-yl)-1H-indole-3-carboxylic acid | 426.86 | 427 (M + 1)⁺ |
| Example 19 | 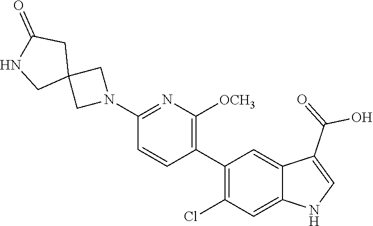 [SMILES: COc1nc(N2CC3(CNC(=O)C3)C2)ccc1-c1cc2c(C(=O)O)c[nH]c2cc1Cl] 6-chloro-5-(2-methoxy-6-(7-oxo-2,6-diazaspiro[3.4] octan-2-yl)pyridin-3-yl)-1H-indole-3-carboxylic acid | 426.86 | 427 (M + 1)⁺ |
| Example 20 | 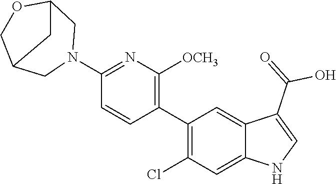 [SMILES: COc1nc(N2CC3COC(C3)C2)ccc1-c1cc2c(C(=O)O)c[nH]c2cc1Cl] racemic-5-(6-(6-oxa-3-azabicyclo[3.2.1]octan-3-yl)-2- methoxypyridin-3-yl)-6-chloro-1H-indole-3-carboxylic acid | 413.86 | 414 (M + 1)⁺ |
| Example 21 | 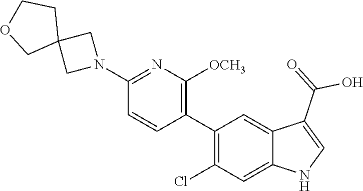 [SMILES: COc1nc(N2CC3(CCOC3)C2)ccc1-c1cc2c(C(=O)O)c[nH]c2cc1Cl] 6-chloro-5-(2-methoxy-6-(6-oxa-2-azaspiro[3.4]octan- 2-yl)pyridin-3-yl)-1H-indole-3-carboxylic acid | 413.86 | 414 (M + 1)⁺ |
| Example 22 | 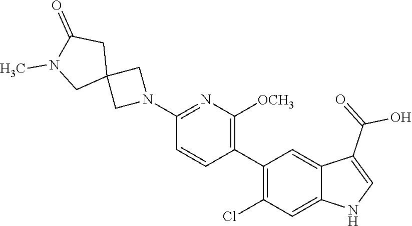 [SMILES: COc1nc(N2CC3(CC(=O)N(C)C3)C2)ccc1-c1cc2c(C(=O)O)c[nH]c2cc1Cl] 6-chloro-5-(2-methoxy-6-(6-methyl-7-oxo-2,6- diazaspiro[3.4]octan-2-yl)pyridin-3-yl)-1H-indole-3- carboxylic acid | 440.88 | 441 (M + 1)⁺ |
| Example 23 | 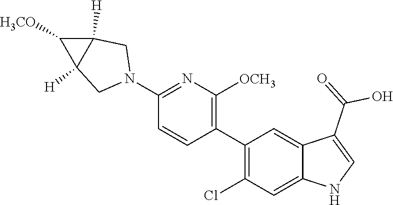 [SMILES: COc1nc(N2C[C@@H]3[C@H](C2)[C@H]3OC)ccc1-c1cc2c(C(=O)O)c[nH]c2cc1Cl] 6-chloro-5-(2-methoxy-6-((trans)-6-methoxy-3- azabicyclo[3.1.0]hexan-3-yl)pyridin-3-yl)-1H-indole- 3-carboxylic acid | 413.86 | 414 (M + 1)⁺ |

### Example 24

### Preparation of 6-chloro-5-(6-((trans)-6-hydroxy-3-azabicyclo[3.1.0]hexan-3-yl)-2-methoxypyridin-3-yl)-1H-indole-3-carboxylic acid

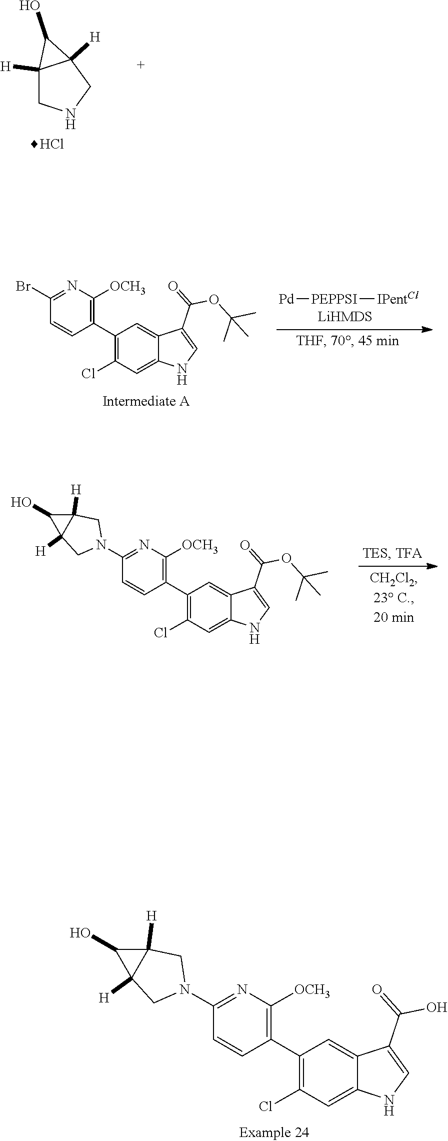

Step 1: Preparation of tert-butyl 6-chloro-5-(6-((trans)-6-hydroxy-3-azabicyclo[3.1.0]hexan-3-yl)-2-methoxypyridin-3-yl)-1H-indole-3-carboxylate: To a glass microwave vial was added Intermediate A (1.0 equiv), trans-3-azabicyclo[3.1.0]hexan-6-ol hydrochloride (3.0 equiv), Pd-PEPPSI-IPent^{Cl} (0.1 equiv), lithium hexamethyldisilazide (1.0 M in THF, 12 equiv) and THF (0.1 M). This mixture was degassed and irradiated in a microwave reactor at 70° C. for 30 minutes. LCMS analysis indicated completion of the reaction. The reaction mixture was cooled to 24° C., loaded onto a 20 g silica gel pre-cartridge, and dried under vacuum. The mixture was purified by column chromatography through silica gel, eluting with 90:10 to 0:100 hexanes:EtOAc as a gradient. The product containing fractions were combined, concentrated, and dried under reduced pressure to afford a solid (35% yield).

Step 2: Preparation of 6-chloro-5-(6-((trans)-6-hydroxy-3-azabicyclo[3.1.0]hexan-3-yl)-2-methoxypyridin-3-yl)-1H-indole-3-carb oxylic acid: To a reaction flask was added tert-butyl 6-chloro-5-(6-((trans)-6-hydroxy-3-azabicyclo[3.1.0]hexan-3-yl)-2-methoxypyridin-3-yl)-1H-indole-3-carboxylate (1.0 equiv), trifluoroacetic acid (50 equiv), triethyl silane (1.5 equiv) and CH₂Cl₂ (0.5 M). The solution was stirred at 24° C. for 20 minutes after which LCMS analysis indicated completion of the reaction. The reaction was concentrated, and the resulting crude reaction mixture was loaded onto a reverse phase pre-cartridge. Purification by reverse phase column chromatography through a C18 column, eluting with 70:30 to 0:100 water:CH₃CN+0.1% formic acid as a gradient. The product containing fractions were combined and lyophilized overnight to yield the title product as a white solid (34% yield). ¹H NMR (400 MHz, d₆-DMSO): δ 11.87 (s, 1H), 8.03 (s, 1H), 7.82 (d, J=7.2 Hz, 1H), 7.54 (s, 1H), 7.28 (d, J=8.0 Hz, 1H), 6.00 (d, J=8.1 Hz, 1H), 5.47 (s, 1H), 3.74 (s, 3H), 3.63 (d, J=10.4 Hz, 2H), 3.42 (d, J=10.1 Hz, 2H), 3.01 (s, 1H), 2.51 (s, 2H). LC-MS (ESI) m/z 400 (M+H)⁺.

The following compounds were prepared in a similar manner as Example 24, by replacing trans-3-azabicyclo[3.1.0]hexan-6-ol hydrochloride with the corresponding secondary amines in the first step. In some cases, the base LiHDMS (1.0 M in THF) in the first step may be replaced with sodium tert-pentoxide.

| Example | Structure | MW | MS (ESI+) |
|---|---|---|---|
| Example 25 | 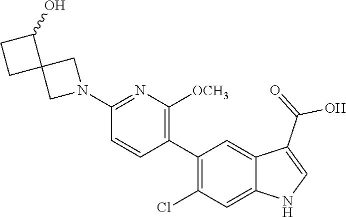 [SMILES: COc1nc(N2CC3(CCC3O)C2)ccc1-c1cc2c(C(=O)O)c[nH]c2cc1Cl] racemic-6-chloro-5-(6-(5-hydroxy-2-azaspiro[3.3] heptan-2-yl)-2-methoxypyridin-3-yl)-1H-indole-3- carboxylic acid | 413.86 | 414 (M + 1)⁺ |
| Example 26 | 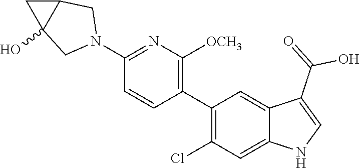 [SMILES: COc1nc(N2CC3CC3(O)C2)ccc1-c1cc2c(C(=O)O)c[nH]c2cc1Cl] racemic-6-chloro-5-(6-(1-hydroxy-3-azabicyclo[3.1.0] hexan-3-yl)-2-methoxypyridin-3-yl)-1H-indole-3- carboxylic acid | 399.83 | 400 (M + 1)⁺ |

### Example 27

### Preparation of 6-chloro-5-(2-methoxy-6-((cis)-tetrahydro-1H-furo[3,4-c]pyrrol-5(3H)-yl)pyridin-3-yl)-1H-indole-3-carboxylic Acid

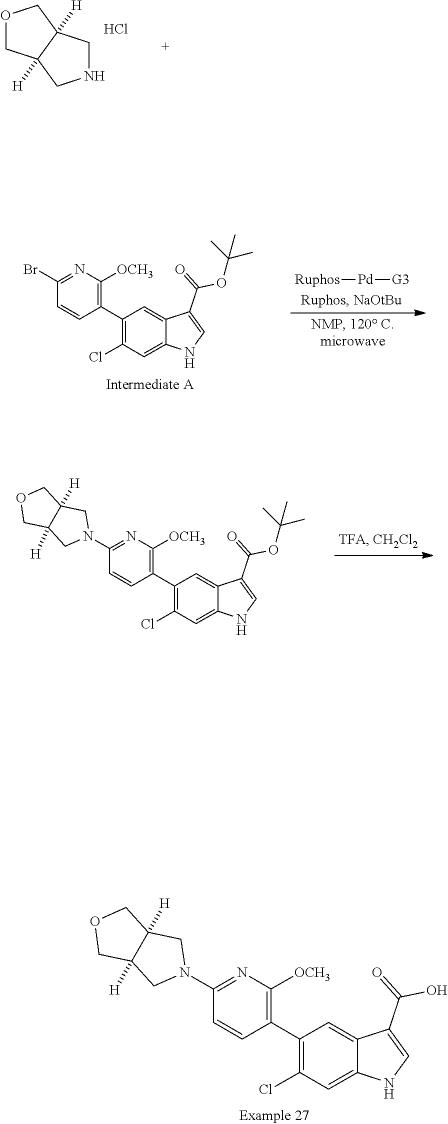

Step 1: Preparation of tert-butyl 6-chloro-5-(2-methoxy-6-((cis)-tetrahydro-1H-furo[3,4-e]pyrrol-5(3H)-yl)pyridin-3-yl)-1H-indole-3-carboxylate: Into a sealed reaction tube equipped with a magnetic stir bar and under nitrogen was added tert-butyl 5-(6-bromo-2-methoxypyridin-3-yl)-6-chloro-1H-indole-3-carboxylate (1.0 equiv), (cis)-hexahydro-1H-furo[3,4-c]pyrrole hydrochloride (1.0 equiv), Ruphos (0.1 equiv), t-BuONa (5.0 equiv), RuPhos-Pd-G3 (0.1 equiv) and NMP (0.08 M). The reaction mixture was irradiated in a microwave reactor for 1 hour at 120° C. The reaction was cooled and quenched with water and the mixture was extracted with ethyl acetate. The combined organic layers were concentrated under reduced pressure. The crude product was purified by preparative HPLC under the following conditions: column, C18 column; mobile phase, 90:10 to 40:60 water:CH₃CN as a gradient over 20 min; detector, UV 220 nm. The product containing fractions were combined, concentrated, and dried under vacuum to afford the title product as a white solid (53% yield).

Step 2: Preparation of 6-chloro-5-(2-methoxy-6-((cis)-tetrahydro-1H-furo[3,4-c]pyrrol-5(3H)-yl)pyridin-3-yl)-1H-indole-3-carboxylic acid: Into a round-bottom flask equipped with a magnetic stir bar was added tert-butyl 6-chloro-5-(2-methoxy-6-((cis)-tetrahydro-1H-furo[3,4-e]pyrrol-5(3H)-yl)pyridin-3-yl)-1H-indole-3-carboxylate (1.0 equiv), TFA (0.3 M) and CH₂Cl₂ (0.07 M). The resulting mixture was stirred at 24° C. for 2 hours. The mixture was concentrated and dried under vacuum. The resulting crude mixture was purified by preparative HPLC under the following conditions: XBridge preparative OBD C18 Column, mobile phase, 13:87 to 37:63 water (+10 mmol/L NH₄HCO₃):CH₃CN as a gradient over 9 min; detector, UV 254 nM. The title product was obtained as a white solid (12% yield). ¹H NMR (300 MHz, d₆-DMSO): δ 11.88 (s, 1H), 8.04 (s, 1H), 7.85 (s, 1H), 7.56 (s, 1H), 7.32 (d, J=8.0 Hz, 1H), 6.10 (d, J=8.0 Hz, 1H), 3.89-3.86 (m, 2H), 3.76 (s, 3H), 3.60-3.58 (m, 4H), 3.46-3.40 (m, 2H), 3.03-2.96 (m, 2H) ppm. LC-MS (ESI) m/z 413 (M+H)⁺.

The following compounds were prepared in a similar manner as Example 27, by replacing (cis)-hexahydro-1H-furo[3,4-c]pyrrole hydrochloride with the corresponding secondary amines in the first step.

| Example | Structure | MW | MS (ESI+) |
|---|---|---|---|
| Example 28 | 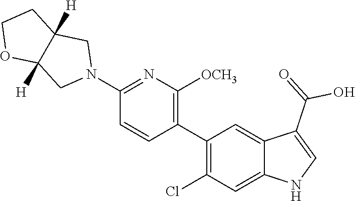 [SMILES: COc1nc(N2C[C@H]3CCO[C@H]3C2)ccc1-c1cc2c(C(=O)O)c[nH]c2cc1Cl] racemic-6-chloro-5-(6-((cis)-hexahydro-5H-furo[2,3-c] pyrrol-5-yl)-2-methoxypyridin-3-yl)-1H-indole-3- carboxylic acid | 413.86 | 414 (M + 1)⁺ |
| Example 29 | 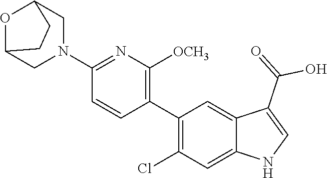 [SMILES: COc1nc(N2CC3CCC(C2)O3)ccc1-c1cc2c(C(=O)O)c[nH]c2cc1Cl] racemic-5-(6-(8-oxa-3-azabicyclo[3.2.1]octan-3-yl)-2- methoxypyridin-3-yl)-6-chloro-1H-indole-3- carboxylic acid | 413.86 | 414 (M + 1)⁺ |
| Example 30 | 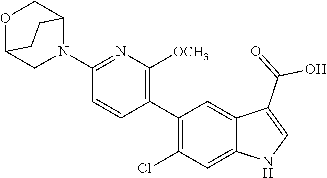 [SMILES: COc1nc(N2CC3CCC2CO3)ccc1-c1cc2c(C(=O)O)c[nH]c2cc1Cl] racemic-5-(6-(2-oxa-5-azabicyclo[2.2.2]octan-5-yl)-2- methoxypyridin-3-yl)-6-chloro-1H-indole-3- carboxylic acid | 413.86 | 414 (M + 1)⁺ |
| Example 31 | 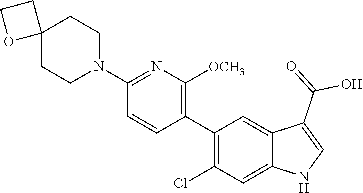 [SMILES: COc1nc(N2CCC3(CCO3)CC2)ccc1-c1cc2c(C(=O)O)c[nH]c2cc1Cl] racemic-6-chloro-5-(2-methoxy-6-(1-oxa-7-azaspiro [3.5]nonan-7-yl)pyridin-3-yl)-1H-indole-3-carboxylic acid | 427.86 | 428 (M + 1)⁺ |

### Example 32

### Preparation of racemic-6-chloro-5-(2-methoxy-6-((trans)tetrahydro-1H-furo[3,4-c]pyrrol-5(3H)-yl)pyridin-3-yl)-1H-indole-3-carboxylic acid

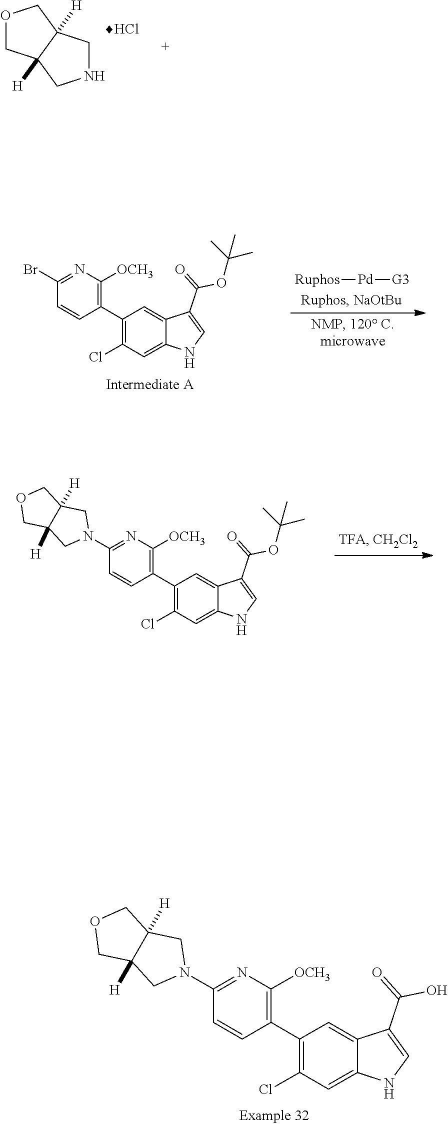

Step 1: Preparation of rac-tert-butyl 6-chloro-5-(2-methoxy-6-((trans)-tetrahydro-1H-furo[3,4-c]pyrrol-5(3H)-yl)pyridin-3-yl)-1H-indole-3-carboxylate: Into a sealed reaction tube equipped with a magnetic stir bar and under nitrogen was added tert-butyl 5-(6-bromo-2-methoxypyridin-3-yl)-6-chloro-1H-indole-3-carboxylate (1.0 equiv), rac-(trans)-hexahydro-1H-furo[3,4-c]pyrrole hydrochloride (1.0 equiv), Ruphos (0.1 equiv), t-BuONa (5.0 equiv), RuPhos-Pd-G3 (0.1 equiv) and NMP (0.08 M). The final reaction mixture was irradiated in a microwave reactor for 1 hour at 120° C. The reaction was cooled and quenched with water and extracted with ethyl acetate and the combined organic layers were concentrated under reduced pressure. The crude product was purified by preparative HPLC under the following conditions: C18 column; mobile phase, 90:10 to 40:60 water:CH₃CN as a gradient over 20 min; detector, UV 220 nm. The product containing fractions were combined, concentrated, and dried under vacuum to afford the title product as a white solid (48% yield).

Step 2: Preparation of racemic-6-chloro-5-(2-methoxy-6-((trans)-tetrahydro-1H-furo[3,4-c]pyrrol-5(3H)-yl)pyridin-3-yl)-1H-indole-3-carboxylic acid: Into a round-bottom flask equipped with a magnetic stir bar was placed rac-tert-butyl 6-chloro-5-(2-methoxy-6-(tetrahydro-1H-furo[3,4-c]pyrrol-5(3H)-yl)pyridin-3-yl)-1H-indole-3-carboxylate (1.0 equiv), TFA (0.3 M) and CH₂Cl₂ (0.07 M). The resulting mixture was stirred at 24° C. for 2 hours. Upon completion of reaction as indicated by LCMS, the reaction mixture was concentrated. The crude product was purified by preparative HPLC under the following conditions: C18 column; mobile phase, 90:10 to 30:70 water:CH₃CN as a gradient over 25 min; detector, UV 254 nm. The title product was obtained as a white solid (96% yield). ¹H NMR (400 MHz, d₆-DMSO): δ 12.07-11.87 (m, 1H), 8.05-8.04 (m, 1H), 7.84-7.83 (m, 1H), 7.56-7.55 (m, 1H), 7.31-7.29 (m, 1H), 6.02-5.91 (m, 1H), 3.99 (s, 2H), 3.86-3.79 (m, 2H), 3.73-3.66 (m, 5H), 3.44-3.38 (m, 2H), 3.14 (s, 2H) ppm. LC-MS (ESI): m/z 413 (M+H)⁺.

### Examples 33 and 34

### Preparation of 6-chloro-5-(2-methoxy-6-((3aR, 6aR)-tetrahydro-1H-furo[3,4-c]pyrrol-5(3H)-yl)pyridin-3-yl)-1H-indole-3-carboxylic acid and 6-chloro-5-(2-methoxy-6-((3aS,6aS)-tetrahydro-1H-furo[3,4-c]pyrrol-5(3H)-yl)pyridin-3-yl)-1H-indole-3-carboxylic acid:

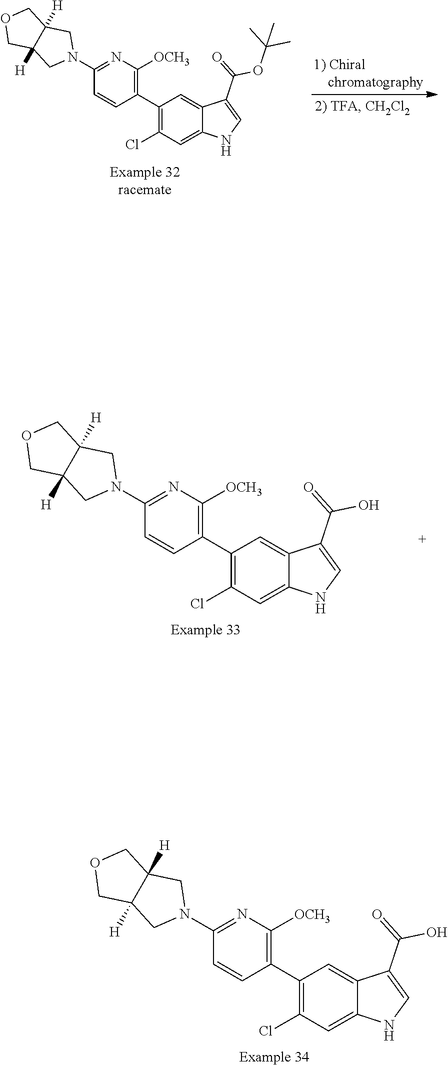 [SMILES: COc1nc(N2C[C@@H]3COC[C@H]3C2)ccc1-c1cc2c(C(=O)OC(C)(C)C)c[nH]c2cc1Cl ; COc1nc(N2C[C@@H]3COC[C@H]3C2)ccc1-c1cc2c(C(=O)O)c[nH]c2cc1Cl ; COc1nc(N2C[C@H]3COC[C@@H]3C2)ccc1-c1cc2c(C(=O)O)c[nH]c2cc1Cl]

Step 1: Preparation of tert-butyl 6-chloro-5-(2-methoxy-6-((3aR,6aR)-tetrahydro-1H-furo[3,4-c]pyrrol-5(3H)-yl)pyridin-3-yl)-1H-indole-3-carboxylate and tert-butyl 6-chloro-5-(2-methoxy-6-((3aR,6aR)-tetrahydro-1H-furo[3,4-c]pyrrol-5(3H)-yl)pyridin-3-yl)-1H-indole-3-carboxylate: racemic-tert-butyl 6-chloro-5-(2-methoxy-6-((trans)-tetrahydro-1H-furo[3,4-c]pyrrol-5(3H)-yl)pyridin-3-yl)-1H-indole-3-carboxylate (Example 32) was separated with chiral preparative-HPLC under the following conditions: column, CHIRAL ART Cellulose-SC, 2×25 cm, 5 mm; mobile phase, 92:8 hexanes (0.5% 2M NH₃ in MeOH):EtOH as an isocratic over 24 min; detector, UV 254 nM. The product containing fractions were combined, concentrated and dried under vacuum to afford tert-butyl 6-chloro-5-(2-methoxy-6-((3aS, 6aS)-tetrahydro-1H-furo[3,4-c]pyrrol-5(3H)-yl)pyridin-3-yl)-1H-indole-3-carboxylate (99% ee) as a white solid and tert-butyl 6-chloro-5-(2-methoxy-6-((3aR, 6aR)-tetrahydro-1H-furo[3,4-c]pyrrol-5(3H)-yl)pyridin-3-yl)-1H-indole-3-carboxylate (99% ee) as a white solid.

Step 2: Preparation of 6-chloro-5-(2-methoxy-6-((3aR, 6aR)-tetrahydro-1H-furo[3,4-c]pyrrol-5(3H)-yl)pyridin-3-yl)-1H-indole-3-carboxylic acid and 6-chloro-5-(2-methoxy-6-((3aS,6aS)-tetrahydro-1H-furo[3,4-e]pyrrol-5(3H)-yl)pyridin-3-yl)-1H-indole-3-carboxylic acid: Into a round-bottom flask equipped with a magnetic stir bar was placed tert-butyl 6-chloro-5-(2-methoxy-6-((3aR,6aR)-tetrahydro-1H-furo [3,4-c]pyrrol-5(3H)-yl)pyridin-3-yl)-1H-indole-3-carboxylate (1.0 equiv), TFA (0.3 M) and CH₂Cl₂ (0.07M). The resulting mixture was stirred at 24° C. for 2 hours. Upon completion of reaction as indicated by LCMS, the reaction mixture was concentrated. The crude product was purified by preparative HPLC under the following conditions: C18 column; mobile phase, 90:10 to 30:70 water:CH₃CN as a gradient over 25 min; detector, UV 254 nm. The title product was obtained as a white solid (95% yield). ¹H NMR (400 MHz, d₆-DMSO): δ 11.88 (d, J=2.9 Hz, 1H), 8.05 (d, J=2.9 Hz, 1H), 7.84 (s, 1H), 7.56 (s, 1H), 7.31 (d, J=8.0 Hz, 1H), 6.03 (d, J=8.1 Hz, 1H), 3.90 (d, J=13.1 Hz, 2H), 3.93-3.90 (m, 5H), 3.46-3.44 (m, 2H), 3.18 (t, J=9.7 Hz, 2H), 2.57-2.55 (m, 2H) ppm. LC-MS (ESI): m/z 413 (M+H)⁺.

6-chloro-5-(2-methoxy-6-((3aS,6aS)-tetrahydro-1H-furo[3,4-c]pyrrol-5(3H)-yl)pyridin-3-yl)-1H-indole-3-carboxylic acid was synthesized in the same manner as described above, starting from tert-butyl 6-chloro-5-(2-methoxy-6-((3aS, 6aS)-tetrahydro-1H-furo[3,4-c]pyrrol-5(3H)-yl)pyridin-3-yl)-1H-indole-3-carboxylate. ¹H NMR (300 MHz, d₆-DMSO): δ 12.06 (s, 1H), 11.89 (d, J=2.9 Hz, 1H), 8.05 (d, J=2.9 Hz, 1H), 7.84 (s, 1H), 7.56 (s, 1H), 7.31 (d, J=8.0 Hz, 1H), 6.03 (d, J=8.1 Hz, 1H), 3.92-3.87 (m, 2H), 3.77-3.75 (m, 5H), 3.48-3.46 (m, 2H), 3.2-3.14 (t, J=9.7 Hz, 2H), 2.60-2.50 (m, 2H) ppm. LC-MS (ESI): m/z 413 (M+H)⁺.

### Example 35

### Preparation of 6-chloro-5-(2-methoxy-6-[8-oxa-2-azaspiro[4.5]decan-2-yl]pyridin-3-yl)-1H-indole-3-carboxylic acid

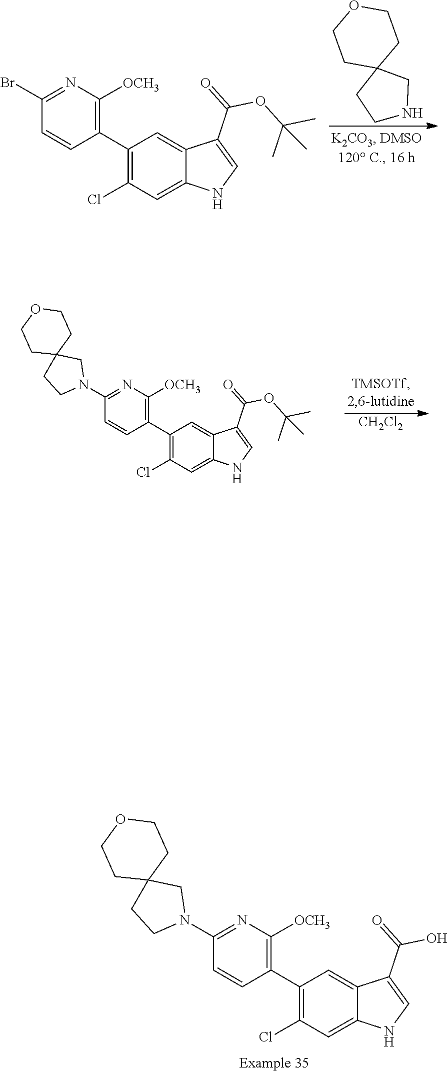

Step 1: Preparation of tert-butyl 6-chloro-5-(2-methoxy-6-[8-oxa-2-azaspiro[4.5]decan-2-yl]pyridin-3-yl)-1H-indole-3-carboxylate: Into a reaction vial equipped with a magnetic stir bar was placed 8-oxa-2-azaspiro[4.5]decane (1.0 equiv), tert-butyl 5-(6-bromo-2-methoxypyridin-3-yl)-6-chloro-1H-indole-3-carboxylate (1.0 equiv), K₂CO₃ (3 equiv) and DMSO (0.18 M). The resulting mixture was stirred at 120° C. for 16 hours. The crude product was purified by preparative HPLC with the following conditions: column, X-select CSH F-phenyl OBD column, 19×250 mm, 5 mm; mobile phase, 25:75 to 15:85 water (10 mmol/L NH₄HCO₃):MeOH as a gradient over 8 min; detector, UV 254 nm. The product containing fractions were combined, concentrated, and dried under vacuum to yield the title product as a white solid (7% yield).

Step 2: Preparation of 6-chloro-5-(2-methoxy-6-[8-oxa-2-azaspiro [4.5]decan-2-yl]pyridin-3-yl)-1H-indole-3-carboxylic acid: Into a reaction vial equipped with a magnetic stir bar was placed tert-butyl 6-chloro-5-(2-methoxy-6-[8-oxa-2-azaspiro [4.5]decan-2-yl]pyridin-3-yl)-1H-indole-3-carboxylate (1.0 equiv) and CH₂Cl₂ (0.11 M). The mixture was cooled to 0° C. in a water/ice bath and treated with drop-wise addition of 2,6-lutidine (30.0 equiv) and TMSOTf (15.0 equiv). The resulting mixture was stirred at 24° C. for 30 minutes. The reaction was then quenched with saturated aqueous NH₄Cl and extracted with ethyl acetate. The combined organic layers were concentrated under reduced pressure. The crude product was purified by preparative HPLC under the following conditions: C18 column; mobile phase, 100:0 to 52:48 water:CH₃CN as a gradient over 20 min; detector, UV 254 nm. The product containing fractions were combined, concentrated, and dried under vacuum to afford the title product as a white solid (18% yield). ¹H-NMR (400 MHz, d₆-DMSO): δ 12.05 (s, 1H), 11.90-11.85 (m, 1H), 8.04 (d, J=2.9 Hz, 1H), 7.84 (s, 1H), 7.56 (s, 1H), 7.30 (d, J=7.9 Hz, 1H), 6.05 (d, J=8.0 Hz, 1H), 3.77 (s, 3H), 3.65-3.60 (m, 5H), 3.52-3.49 (m, 3H), 1.93-1.89 (m, 2H), 1.58-1.57 (m, 4H) ppm. LC-MS (ESI): m/z 441 (M+H)⁺.

The following compounds were prepared in a similar manner as Example 35, by replacing 8-oxa-2-azaspiro[4.5]decane with the corresponding secondary amines in the first step.

| | | | MS |
|---|---|---|---|
| Example | Structure | MW | (ESI+) |
| Example 36 | 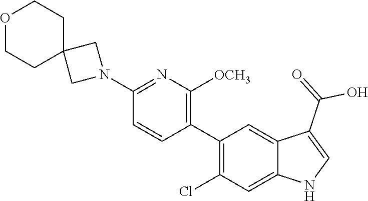 [SMILES: COc1nc(N2CC3(CCOCC3)C2)ccc1-c1cc2c(C(=O)O)c[nH]c2cc1Cl] 6-chloro-5-(2-methoxy-6-(7-oxa-2-azaspiro[3.5]nonan- 2-yl)pyridin-3-yl)-1H-indole-3-carboxylic acid | 427.89 | 428 (M + 1)⁺ |
| Example 37 | 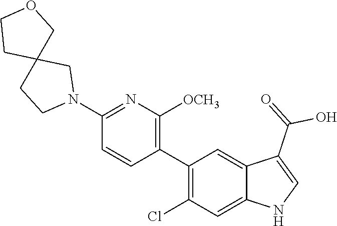 [SMILES: COc1nc(N2CCC3(CCOC3)C2)ccc1-c1cc2c(C(=O)O)c[nH]c2cc1Cl] racemic-6-chloro-5-(2-methoxy-6-(2-oxa-7-azaspiro [4.4]nonan-7-yl)pyridin-3-yl)-1H-indole-3-carboxylic acid | 427.89 | 428 (M + 1)⁺ |
| Example 38 | 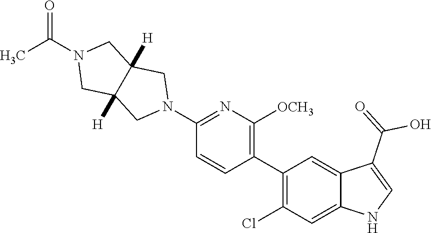 [SMILES: COc1nc(N2C[C@H]3CN(C(C)=O)C[C@H]3C2)ccc1-c1cc2c(C(=O)O)c[nH]c2cc1Cl] 5-(6-((3aR,6aS)-5-acetylhexahydropyrrolo [3,4-c]pyrrol-2(1H)-yl)-2-methoxypyridin-3-yl)-6- chloro-1H-indole-3-carboxylic acid | 454.91 | 455 (M + 1)⁺ |
| Example 39 | 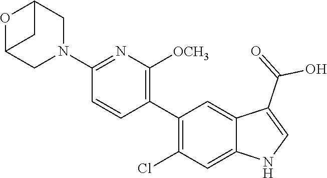 [SMILES: COc1nc(N2CC3CC(C2)O3)ccc1-c1cc2c(C(=O)O)c[nH]c2cc1Cl] 5-(6-(6-oxa-3-azabicyclo[3.1.1]heptan-3-yl)-2-methoxypyridin- 3-yl)-6-chloro-1H-indole-3-carboxylic acid | 399.83 | 400 (M + 1)⁺ |

### Example 40

### Preparation of 6-fluoro-5-(2-methoxy-6-[2-oxa-6-azaspiro[3.3]hentan-6-yl]pyridin-3-yl)-1H-indole-3-carboxylic Acid

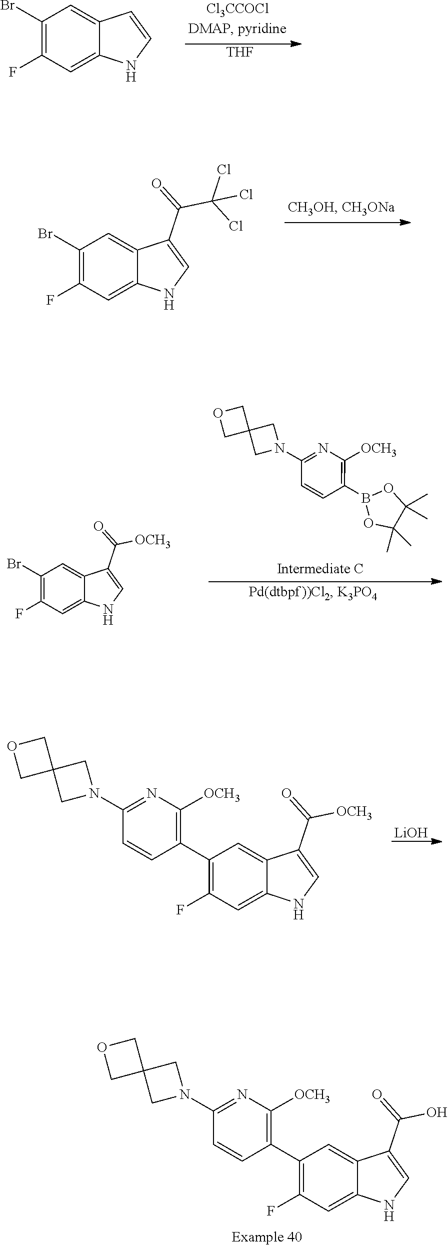

Step 1: Preparation of 1-(5-bromo-6-fluoro-1H-indol-3-yl)-2,2,2-trichloroethanone: Into a round bottom flask equipped with a magnetic stir bar was added 5-bromo-6-fluoro-1H-indole (1.0 equiv), THF (0.26 M), DMAP (0.1 equiv) and pyridine (2.4 equiv). This was followed by the addition of trichloroacetyl chloride (2.6 equiv) at 0° C. The resulting solution was heated for 16 hours at 60° C. The reaction was cooled and then quenched by water. The resulting solution was extracted with ethyl acetate and the organic layers were combined and concentrated. The residue was applied onto a silica gel column and eluted with 20:80 EtOAc:petroleum ether to afford the title product as a yellow solid (74% yield).

Step 2: Preparation of 5-bromo-6-fluoro-1H-indole-3-carboxylate: Into a round bottom flask equipped with a magnetic stir bar was added 1-(5-bromo-6-fluoro-1H-indol-3-yl)-2,2,2-trichloroethanone (1.0 equiv), CH₃OH (0.2 M) and CH₃ONa (2.0 equiv). The resulting solution was heated to 45° C. for 2 hours. The reaction cooled and then quenched with water. The resulting solution was extracted with ethyl acetate and the organic layers were combined and concentrated. The residue was applied onto a silica gel column and eluted with 25:75 EtOAc:petroleum ether to afford methyl 5-bromo-6-fluoro-1H-indole-3-carboxylate as a light yellow solid (79% yield).

Step 3: Preparation of methyl 6-fluoro-5-(2-methoxy-6-[2-oxa-6-azaspiro[3.3]heptan-6-yl]pyridin-3-yl)-1H-indole-3-carboxylate: Into a round bottom flask equipped with a magnetic stir bar and purged and maintained under nitrogen atmosphere was placed methyl 5-bromo-6-fluoro-1H-indole-3-carboxylate (1.0 equiv), 6-[6-methoxy-5-(4,4,5,5-tetramethyl-1,3,2-dioxaborolan-2-yl)pyridin-2-yl]-2-oxa-6-azaspiro[3.3]heptane (Intermediate C, 1.2 equiv), Pd(dtbpf)Cl₂ (0.1 equiv), K₃PO₄ (2 equiv), dioxane (0.08 M) and H₂O (0.02 M). The resulting mixture was heated to 80° C. for 4 hours. The reaction was cooled, filtered and the resulting filtrate was concentrated. The crude product was purified by preparative HPLC with the following conditions: C18 column; mobile phase, 70:30 to 30:70 water:CH₃CN as a gradient over 20 min. The title product was obtained as a light yellow solid (74% yield).

Step 4: Preparation of 6-fluoro-5-(2-methoxy-6-[2-oxa-6-azaspiro[3.3]heptan-6-yl]pyridin-3-yl)-1H-indole-3-carboxylic acid: Into a round bottom flask equipped with a magnetic stir bar was added methyl 6-fluoro-5-(2-methoxy-6-[2-oxa-6-azaspiro[3.3]heptan-6-yl]pyridin-3-yl)-1H-indole-3-carboxylate (1.0 equiv), lithium hydroxide (15.0 equiv), THF (0.15 M), MeOH (0.15 M) and H₂O (0.15 M). The resulting solution was heated to 60° C. for 16 hours. The pH value of the solution was adjusted to 7 with 1 M aqueous HCl. The resulting solution was extracted with ethyl acetate and the organic layers were combined and concentrated. The crude product was purified by preparative HPLC with the following conditions: C18 column; mobile phase, 90:10 to 52:48 water:CH₃CN as a gradient over 20 min. The title product was obtained as a white solid (21% yield). ¹H NMR (400 MHz, d₆-DMSO): δ 11.97 (s, 1H), 11.82 (d, J=2.7 Hz, 1H), 8.00 (d, J=2.6 Hz, 1H), 7.83 (d, J=7.5 Hz, 1H), 7.42 (d, J=7.9 Hz, 1H), 7.26 (d, J=10.4 Hz, 1H), 6.03 (d, J=7.9 Hz, 1H), 4.75 (s, 4H), 4.15 (s, 4H), 3.77 (s, 3H). LC-MS (ESI) m/z 383 (M+H)⁺.

### Example 41

### Preparation of 4,6-difluoro-5-(2-methoxy-6-(2-oxa-6-azaspiro[3.3]heptan-6-yl)pyridin-3-yl)-1H-indole-3-carboxylic acid

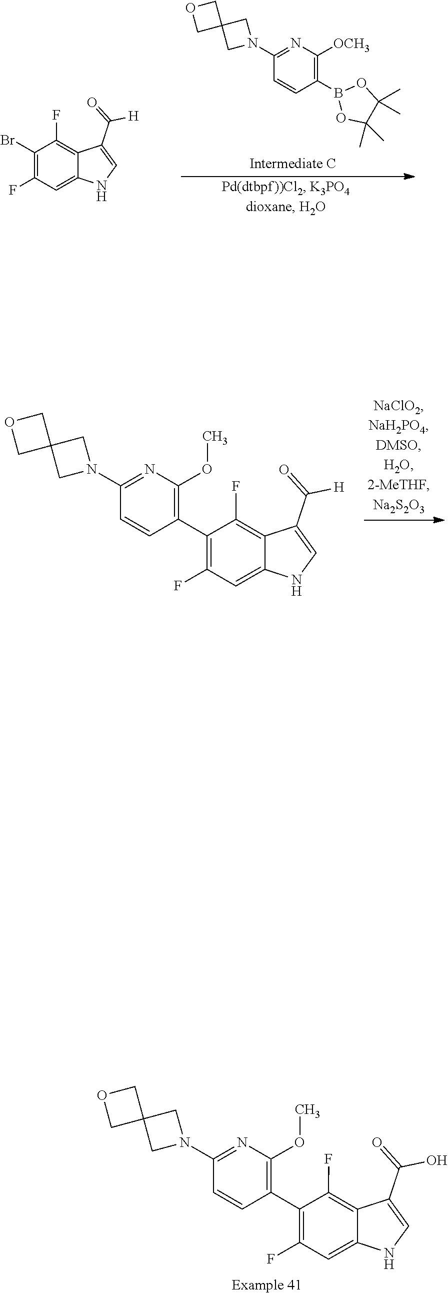 [SMILES: O=Cc1c[nH]c2cc(F)c(Br)c(F)c12 ; COc1nc(N2CC3(COC3)C2)ccc1B1OC(C)(C)C(C)(C)O1 ; COc1nc(N2CC3(COC3)C2)ccc1-c1c(F)cc2[nH]cc(C=O)c2c1F ; COc1nc(N2CC3(COC3)C2)ccc1-c1c(F)cc2[nH]cc(C(=O)O)c2c1F]

Step 1: Preparation of 4,6-difluoro-5-(2-methoxy-6-(2-oxa-6-azaspiro [3.3]heptan-6-yl)pyridin-3-yl)-1H-indole-3-carbaldehyde

Into a round bottom flask equipped with a magnetic stir bar and maintained under nitrogen atmosphere was added ethyl 6-[6-methoxy-5-(4,4,5,5-tetramethyl-1,3,2-dioxaborolan-2-yl)pyridin-2-yl]-2-oxa-6-azaspiro[3.3]heptane (Intermediate C, 1.0 equiv), 5-bromo-4,6-difluoro-1H-indole-3-carbaldehyde (1.0 equiv), Pd(dtbpf)Cl₂ (0.1 equiv), Cs₂CO₃ (3.0 equiv), dioxane (0.3 M) and H₂O (0.075 M). The resulting solution was heated to 80° C. for 4 hours under a nitrogen atmosphere. LC-MS analysis indicated consumption of starting material and formation of product. The reaction was cooled and quenched with water at 24° C. The resulting mixture was poured into a separatory funnel and the aqueous layer was extracted with ethyl acetate. The combined organics were dried over anhydrous Na₂SO₄, filtered, and concentrated under reduced pressure. The residue was purified by silica gel column chromatography, eluting with 50:50 petroleum ether/EtOAc to afford the title product as a brownish yellow solid (22% yield).

Step 2: Preparation of 4,6-difluoro-5-(2-methoxy-6-(2-oxa-6-azaspiro [3.3]heptan-6-yl)pyridin-3-yl)-1H-indole-3-carboxylic acid: Into a round bottom flask equipped with a magnetic stir bar was added 4,6-difluoro-5-(2-methoxy-6-[2-oxa-6-azaspiro[3.3]heptan-6-yl]pyridin-3-yl)-1H-indole-3-carbaldehyde (1.0 equiv), NaClO₂ (5.0 equiv), DMSO (0.04 M) and 2-methyltetrahydrofuran (0.08 M). The mixture was cooled to 0° C. at which time NaH₂PO₄ (5.0 equiv) in H₂O (0.04 M) was added dropwise at 0° C. The resulting mixture was stirred for 2 hours at 24° C. LC-MS analysis indicated consumption of starting material and formation of product. The reaction was quenched with water and the resulting mixture was poured into a separatory funnel and the aqueous layer was extracted with ethyl acetate and dried over anhydrous Na₂SO₄. After filtration, the filtrate was concentrated under reduced pressure. The crude product was purified by preparative HPLC with the following conditions:) (Bridge preparative OBD C18 column, 30×150 mm, 5 mm; mobile phase, 85:15 to 70:30 water (+10 mmol/L NH₄HCO₃):CH₃CN as a gradient over 8 min; detector, UV 254 nm. The title product was obtained as a white solid (4% yield). ¹H NMR (400 MHz, CD₃OD): δ 7.91 (s, 1H), 7.39 (d, J=7.8 Hz, 1H), 7.03 (d, J=9.0 Hz, 1H), 6.05 (d, J=7.9 Hz, 1H), 4.89 (s, 4H), 4.21 (s, 4H), 3.85 (s, 3H) ppm. LC-MS (ESI) m/z 401 (M+H)⁺.

### Example 42

### AMPKα1β1γ1 and AMPKα1β2γ1 Biochemical Assay

Compounds disclosed herein were evaluated for their ability to activate AMPKα1β1γ1 by measuring phosphorylation of a commercially available fluorescent AMPK substrate using the LabChip® EZ Reader microfluidic system that is based on a mobility shift principle in which unphosphorylated fluorescent substrates may be separated and distinguished from phosphorylated fluorescent substrates due to the difference in negative charge. Briefly, compounds were serial diluted 3-fold in 100% DMSO for an 11-point dose-response curve. A 20 nL aliquot of diluted compound was transferred into a 384-well plate using a liquid handler (Labcyte Inc., Echo 550). A pre-prepared solution (10 mM MgCl₂, 0.01% Brij-35, 2 mM DTT, 0.05% BSA, 1 mM EGTA, and 50 mM HEPES pH 7.5) was combined with 0.83 nM AMPKα1β1γ1 (Carna Biosciences, 02-113). The enzyme solution (15 μL) was aliquoted into each well and the plate was incubated at 24° C. for 30 min. A second stock solution (10 mM MgCl₂, 0.01% Brij-35, 2 mM DTT, 0.05% BSA, 1 mM EGTA, 800 μM ATP, and 50 mM HEPES pH 7.5) was combined with 6 μM fluorescent peptide substrate for AMPKal (Perkin Elmer, 760351). The kinase reaction was initiated by adding 5 μL of the second solution containing substrate and ATP to each well. The plate was incubated at 24° C. for 90 minutes and 75 μL of 0.5 M EDTA was added to terminate the reaction. The plates were analyzed using a LabChip® EZ Reader (Perkin Elmer, 122919) and the effective concentration to achieve 50% of the maximal activity (EC₅₀) was calculated after normalizing to the DMSO vehicle control.

The same procedure above was also used to measure activation of the AMPKα1β2γ1 isoform, replacing AMPKα1β1γ1 with AMPKα1β2γ1 Biosciences 02-147). Table 1 below shows the EC₅₀ values obtained for the compounds disclosed herein. EC50 values are categorized according to potency: A≤50 nM; 50 nM<B≤100 nM; 100 nM<C≤250 nM; 250 nM<D≤10 μM; E>10 μM; and “-” means “not determined”.

TABLE 1

| | Human AMPK | Human AMPK |
|---|---|---|
| | AMPKα1β1γ1 EC₅₀ | AMPKα1β1γ1 EC₅₀ |
| Examples | (nM) | (nM) |
| Example 1 | A | E |
| Example 2 | C | — |
| Example 3 | A | D |
| Example 4 | A | E |
| Example 5 | A | E |
| Example 6 | B | E |
| Example 7 | A | — |
| Example 8 | A | E |
| Example 9 | A | E |
| Example 10 | A | E |
| Example 11 | B | — |
| Example 12 | A | E |
| Example 13 | A | E |
| Example 14 | B | — |
| Example 15 | B | E |
| Example 16 | A | — |
| Example 17 | B | E |
| Example 18 | A | E |
| Example 19 | A | E |
| Example 20 | B | — |
| Example 21 | A | E |
| Example 22 | A | E |
| Example 23 | B | E |
| Example 24 | A | D |
| Example 25 | B | E |
| Example 26 | A | E |
| Example 27 | B | E |
| Example 28 | A | E |
| Example 29 | C | — |
| Example 30 | C | — |
| Example 31 | B | — |
| Example 32 | A | — |
| Example 33 | A | D |
| Example 34 | A | D |
| Example 35 | C | — |
| Example 36 | C | — |
| Example 37 | C | — |
| Example 38 | A | D |
| Example 39 | A | — |
| Example 40 | C | — |
| Example 41 | A | E |

The embodiments described above are intended to be merely exemplary, and those skilled in the art will recognize, or will be able to ascertain using no more than routine experimentation, numerous equivalents of specific compounds, materials and procedures. All such equivalents are considered to be within the scope of the claimed subject matter and are encompassed by the appended claims. Since modifications will be apparent to those of skill in the art, it is intended that the claimed subject matter be limited only by the scope of the appended claims.

1. A compound of Formula (I): 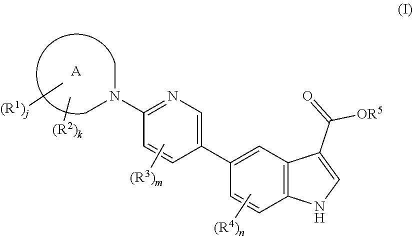
or a pharmaceutically acceptable salt, solvate, hydrate, a single stereoisomer or a mixture of stereoisomers or an isotopic variant, wherein:
Ring A is a bicyclic heterocyclyl;
each R¹ is independently alkyl, haloalkyl, cyanoalkyl, cycloalkyl, heterocyclyl, heterocyclylalkyl —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)NR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl, is optionally substituted with halo, alkyl, haloalkyl, alkoxy, haloalkoxy, hydroxyl or amino;
each R² is independently halo, oxo, cyano, alkyl, haloalkyl, cyanoalkyl, cycloalkyl, cycloalkylalkyl, heterocyclyl, heterocyclylalkyl, —R^{u}OR⁶, —R^{u}NR⁷R⁸, —R^{u}C(O)OR⁹, —R^{u}C(O)NR⁷R⁸, —R^{u}C(O)R¹¹, —R^{u}NR¹⁰C(O)R¹¹, —R^{u}NR¹⁰C(O)OR⁹, —R^{u}S(O)ₜNR⁷R⁸, —R^{u}S(O)ₜR¹², or —R^{u}NR¹⁰S(O)ₜR¹² wherein the cycloalkyl, cycloalkylalkyl, heterocyclyl or heterocyclylalkyl is optionally substituted with halo, alkyl, haloalkyl, alkoxy, haloalkoxy, hydroxyl or amino;
each R³ is independently halo, alkyl, haloalkyl, alkoxy, haloalkoxy, alkylamino, haloalkylamino, alkylthio or haloalkylthio;
each R⁴ is independently halo, alkyl, haloalkyl or cyano;
R⁵ is hydrogen or a group converted to hydrogen in vivo, which comprises alkyl, aryl, aralkyl, —C(R^{b})(R^{c})OC(O)R^{v}, —C(R^{b})(R^{c})OC(O)OR^{v}, 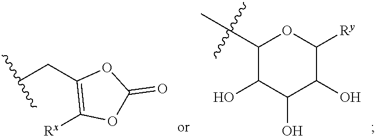
each R⁶ is independently hydrogen, alkyl, haloalkyl, cycloalkyl, heterocyclyl, aryl or heteroaryl wherein the cycloalkyl, heterocyclyl, aryl or heteroaryl is optionally substituted with halo, alkyl, haloalkyl, alkoxy, haloalkoxy or hydroxyl;
R⁷ and R⁸ are selected from (i) or (ii):
R⁷ and R⁸ are each independently hydrogen, alkyl, haloalkyl or alkoxyalkyl; or
R⁷ and R⁸, together with the nitrogen atom to which they are attached, form heterocylyl optionally substituted with halo, oxo, amino, alkyl, haloalkyl, alkoxy, haloalkoxy, hydroxyl or amino;
R⁹ and R¹⁰ are each independently hydrogen, alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
R¹¹ and R¹² are each independently alkyl, haloalkyl, alkoxyalkyl, haloalkoxyalkyl, cycloalkyl, cycloalkylalkyl, heterocyclyl, heterocyclylalkyl, aryl, aralkyl, heteroaryl or heteroaralkyl;
each R^{u} is independently a direct bond or alkylene;
R^{b}, R^{c}, and R^{x} are each independently hydrogen, alkyl, haloalkyl, cyanoalkyl, cycloalkyl, aryl or aralkyl;
R^{v} is alkyl, haloalkyl, cycloalkyl, aryl or aralkyl;
R^{y} is —C(O)OR^{z} or —CH₂OR^{z};
R^{z} is hydrogen or alkyl;
j and k are each independently 0, 1, 2 or 3;
m is 1, 2 or 3;
n is 1 or 2; and
each t is independently 0, 1 or 2.

2. The compound of claim 1 having the Formula (Ia): 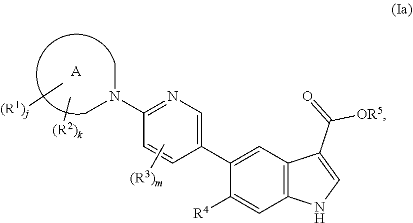
wherein Ring A, R¹, R², R³, R⁴, R⁵, j, k and m are as defined in claim 1.

3. The compound of claim 1 having the Formula (Ib): 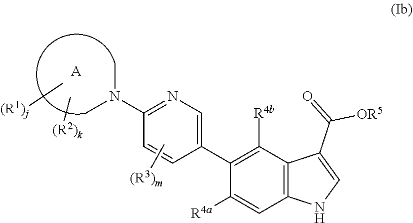
wherein R^{4a} and R^{4b} are each independently halo, alkyl, haloalkyl or cyano and Ring A, R¹, R², R³, R⁵, j, k and m are as defined in claim 1.

4. The compound of claim 1 having the Formula (Ic): 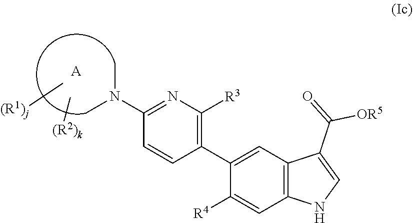
wherein Ring A, R¹, R², R³, R⁴, R⁵, j, and k are as defined in claim 1.

5. The compound of claim 1 the Formula (Id): 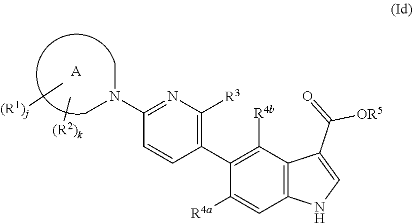
wherein R^{4a} and R^{4b} are each independently halo, alkyl, haloalkyl or cyano and Ring A, R¹, R², R³, R⁵, j and k are as defined in claim 1.

6.-12. (canceled)

13. The compound of claim 1 wherein Ring A is 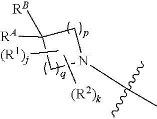
R^{A} and R^{B}, together with the carbon atom to which they are attached, form cycloalkyl or heterocyclyl;
p is 0, 1, 2 or 3;
q is 0, 1, 2 or 3, provided the sum of p and q are 2, 3, 4 or 5; and
wherein j, k, R¹ and R² are as described in claim 1 and R¹ and R² may be on either ring of the spirocycle.

14. The compound of claim 1 wherein Ring A is 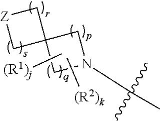
wherein Z is —N(R^{C})—, —N(R^{G})S(O)ₜ—, —O—, —S(O)ₜ—, or —C(R^{E})(R^{F})—;
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q is 2, 3, 4 or 5;
r is 0, 1, 2 or 3 and s is 0, 1, 2 or 3, provided the sum of r and s is 2, 3, 4 or 5;
t is 0, 1 or 2;
and wherein j, k, R¹ and R² are as described in claim 1 and R¹ and R² may be on either ring of the spirocycle.

15. The compound of claim 14 wherein Ring A is selected from: 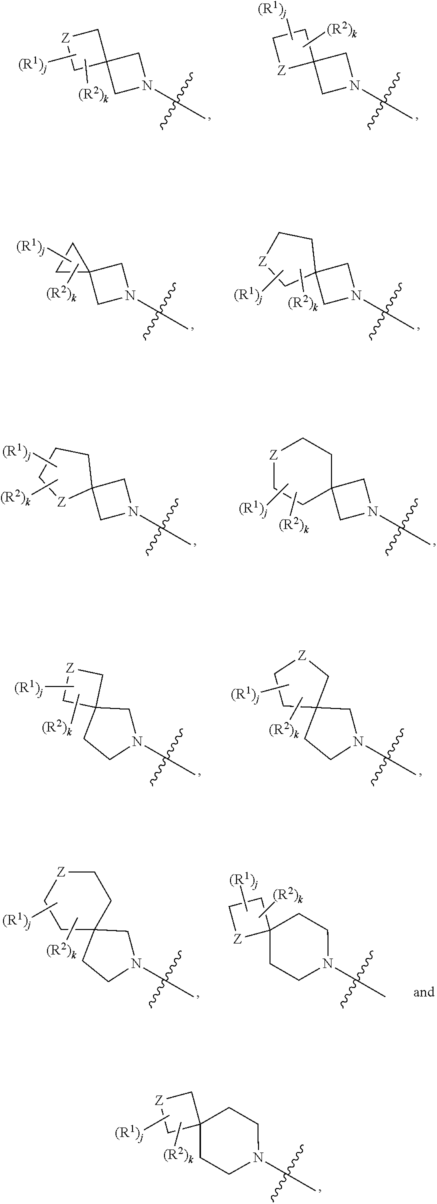
wherein Z, j, k, R¹ and R² are as described in claim 14.

16. The compound of claim 14 wherein Ring A is selected from: 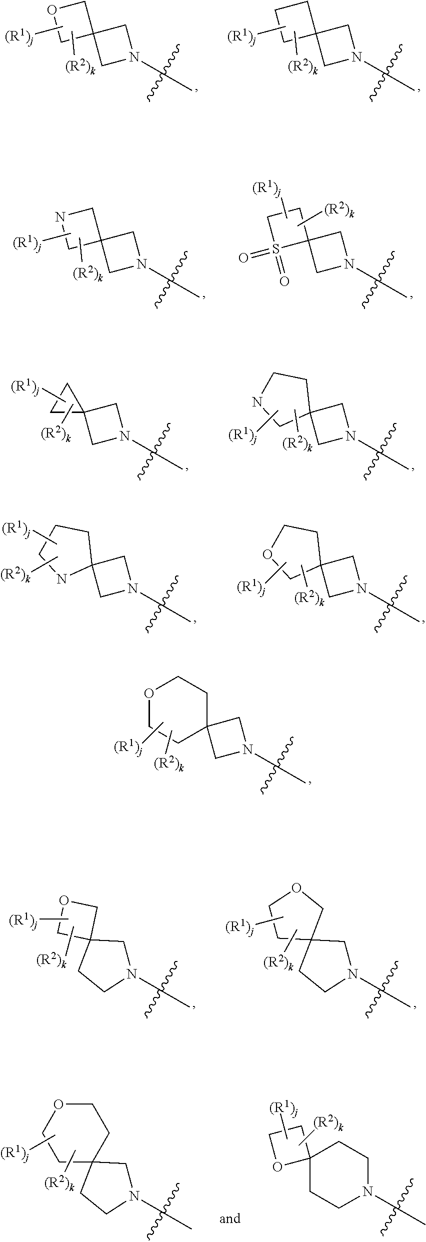
wherein R¹, R², j and k are as described in claim 14.

17.-23. (canceled)

24. The compound of claim 1 wherein the bicyclic heterocyclyl of Ring A is a fused bicyclic heterocyclyl.

25. The compound of claim 1 wherein Ring A is 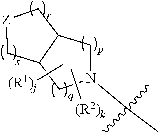
Z is —N(R^{C})—, —N(R^{G})S(O)ₜ—, —O—, —S(O)ₜ—, or —C(R^{E})(R^{F})—,
R^{C} is hydrogen or R¹;
R^{E} and R^{F} are each independently hydrogen or R²;
R^{G} is hydrogen or alkyl;
p is 0, 1, 2 or 3 and q is 0, 1, 2 or 3, provided the sum of p and q are 2, 3, 4 or 5;
r is 0, 1, 2 or 3 and s is 0, 1, 2 or 3, provided the sum of r and s are 1, 2, 3, 4 or 5 or when Z is —C(R^{E})(R^{F})—, the sum of r and s may additionally be 0;
t is 0, 1 or 2; and
wherein j, k, R¹ and R² are as described in claim 1 and R¹ and R² may be on either ring of the fused bicyclic heterocyclyl.

26.-37. (canceled)

38. The compound claim 1 wherein Ring A is a bridged heterocyclyl.

39. The compound of claim 1 wherein Ring A is 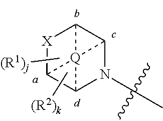
wherein Q is —(CH₂)_{g}—, —CH₂YCH₂—, or —(CH₂)ₕY—, wherein —(CH₂)_{g}—, —CH₂YCH₂—, or —(CH₂)ₕY— is attached to the ring carbon atoms at a and b, c and d, a and c, or b and d;
Y is —O—, —S(O)ₜ—, or —N(R^{C})—;
X is —N(R^{C})—, —C(R^{E})(R^{F})—, —O— or —S(O)ₜ—;
g is 1, 2 or 3;
h is 0, 1 or 2;
each R^{C} is independently hydrogen or R¹;
R^{E} and R^{F} are each independently hydrogen or R²;
j and k are each independently 0, 1, 2 or 3;
t is 0, 1 or 2; and
wherein each R¹ and R² is as described in claim 1.

40.-54. (canceled)

55. The compound of claim 54 wherein R^{4b} is chloro.

56. The compound of claim 1 wherein the compound is selected from:
6-chloro-5-(2-methoxy-6-(2-oxa-6-azaspiro[3.3]heptan-6-yl)pyridin-3-yl)-1H-indole-3-carboxylic acid; [Ex 1]
5-(6-((1S, 4S)2-oxa-5-azabicyclo[2.2.1]heptan-5-yl)-2-methoxypyridin-3-yl)-6-chloro-1H-indole-3-carboxylic acid;
5-(6-((1R, 4R)2-oxa-5-azabicyclo[2.2.1]heptan-5-yl)-2-methoxypyridin-3-yl)-6-chloro-1H-indole-3-carboxylic acid;
5-(6-(2-oxa-5-azabicyclo[2.2.1]heptan-5-yl)-2-methoxypyridin-3-yl)-6-chloro-1H-indole-3-carboxylic acid; [Ex 2]
6-chloro-5-(6-(6-(hydroxymethyl)-3-azabicyclo[3.1.0]hexan-3-yl)-2-methoxypyridin-3-yl)-1H-indole-3-carboxylic acid;
6-chloro-5-(6-((cis)-6-(hydroxymethyl)-3-azabicyclo[3.1.0]hexan-3-yl)-2-methoxypyridin-3-yl)-1H-indole-3-carboxylic acid;
6-chloro-5-(6-((trans)-6-(hydroxymethyl)-3-azabicyclo[3.1.0]hexan-3-yl)-2-methoxypyridin-3-yl)-1H-indole-3-carboxylic acid; [Ex 3]
6-chloro-5-(6-(6-hydroxy-2-azaspiro[3.3]heptan-2-yl)-2-methoxypyridin-3-yl)-1H-indole-3-carboxylic acid; [Ex 4]
6-chloro-5-(2-methoxy-6-(2-oxa-6-azaspiro[3.4]octan-6-yl)pyridin-3-yl)-1H-indole-3-carboxylic acid; [Ex 5]
6-chloro-5-(2-methoxy-6-(2,6-diazaspiro[3.3]heptan-2-yl)pyridin-3-yl)-1H-indole-3-carboxylic acid; [Ex 6]
5-(6-(6-((tert-butoxycarbonyl)amino)-2-azaspiro[3.3]heptan-2-yl)-2-methoxypyridin-3-yl)-6-chloro-1H-indole-3-carboxylic acid; [Ex 7]
6-chloro-5-(2-methoxy-6-(5-oxa-2-azaspiro[3.4]octan-2-yl)pyridin-3-yl)-1H-indole-3-carboxylic acid; [Ex 8]
6-chloro-5-(2-methoxy-6-(6-(methoxymethyl)-3-azabicyclo[3.1.0]hexan-3-yl)pyridin-3-yl)-1H-indole-3-carboxylic acid;
6-chloro-5-(2-methoxy-6-((cis)-6-(methoxymethyl)-3-azabicyclo[3.1.0]hexan-3-yl)pyridin-3-yl)-1H-indole-3-carboxylic acid;
6-chloro-5-(2-methoxy-6-((trans)-6-(methoxymethyl)-3-azabicyclo[3.1.0]hexan-3-yl)pyridin-3-yl)-1H-indole-3-carboxylic acid; [Ex 9]
5-(6-(6-amino-2-azaspiro[3.3]heptan-2-yl)-2-methoxypyridin-3-yl)-6-chloro-1H-indole-3-carboxylic acid; [Ex 10]
6-chloro-5-(2-methoxy-6-(2, 6-diazaspiro[3.3]heptan-2-yl)pyridin-3-yl)-1H-indole-3-carboxylic acid; [Ex 11]
5-[6-(5-azaspiro[2.3]hexan-5-yl)-2-methoxy-3-pyridyl]-6-chloro-1H-indole-3-carboxylic acid; [Ex 12]
6-chloro-5-(2-methoxy-6-(2-azaspiro[3.3]heptan-2-yl)pyridin-3-yl)-1H-indole-3-carboxylic acid; [Ex 13]
5-(6-(3-azabicyclo[3.1.0]hexan-3-yl)-2-methoxypyridin-3-yl)-6-chloro-1H-indole-3-carboxylic acid; [Ex 14]
6-chloro-5-(6-(1-cyano-3-azabicyclo[3.1.0]hexan-3-yl)-2-methoxypyridin-3-yl)-1H-indole-3-carboxylic acid; [Ex 15]
6-chloro-5-(6-((1R)1-cyano-3-azabicyclo[3.1.0]hexan-3-yl)-2-methoxypyridin-3-yl)-1H-indole-3-carboxylic acid;
6-chloro-5-(6-((1S)1-cyano-3-azabicyclo [3.1.0]hexan-3-yl)-2-methoxypyridin-3-yl)-1H-indole-3-carboxylic acid;
6-chloro-5-(6-(1,1-dioxido-1-thia-6-azaspiro [3.3]heptan-6-yl)-2-methoxypyridin-3-yl)-1H-indole-3-carboxylic acid; [Ex 16]
6-chloro-5-(2-methoxy-6-(6-oxo-2,5-diazaspiro[3.4]octan-2-yl)pyridine-3-yl)-1H-indole-3-carboxylic acid; [Ex 17]
6-chloro-5-(2-methoxy-6-(5-oxo-2,6-diazaspiro[3.4]octan-2-yl)pyridin-3-yl)-1H-indole-3-carboxylic acid; [Ex 18]
6-chloro-5-(2-methoxy-6-(7-oxo-2,6-diazaspiro[3.4]octan-2-yl)pyridin-3-yl)-1H-indole-3-carboxylic acid; [Ex 19]
5-(6-(6-oxa-3-azabicyclo[3.2.1]octan-3-yl)-2-methoxypyridin-3-yl)-6-chloro-1H-indole-3-carboxylic acid; [Ex 20]
5-(6-(1R,5S)-(6-oxa-3-azabicyclo[3.2.1]octan-3-yl)-2-methoxypyridin-3-yl)-6-chloro-1H-indole-3-carboxylic acid;
5-(6-(1S,5R)-(6-oxa-3-azabicyclo[3.2.1]octan-3-yl)-2-methoxypyridin-3-yl)-6-chloro-1H-indole-3-carboxylic acid;
6-chloro-5-(2-methoxy-6-(6-oxa-2-azaspiro[3.4]octan-2-yl)pyridin-3-yl)-1H-indole-3-carboxylic acid; [Ex 21]
6-chloro-5-(2-methoxy-6-(6-methyl-7-oxo-2,6-diazaspiro[3.4]octan-2-yl)pyridine-3-yl)-1H-indole-3-carboxylic acid; [Ex 22]
6-chloro-5-(2-methoxy-6-(6-methoxy-3-azabicyclo[3.1.0]hexan-3-yl)pyridin-3-yl)-1H-indole-3-carboxylic acid;
6-chloro-5-(2-methoxy-6-((1R,5S,6r)-6-methoxy-3-azabicyclo[3.1.0]hexan-3-yl)pyridin-3-yl)-1H-indole-3-carboxylic acid;
6-chloro-5-(2-methoxy-6-((1R,5S,6s)-6-methoxy-3-azabicyclo[3.1.0]hexan-3-yl)pyridin-3-yl)-1H-indole-3-carboxylic acid; [Ex 23]
6-chloro-5-(6-(6-hydroxy-3-azabicyclo[3.1.0]hexan-3-yl)-2-methoxypyridin-3-yl)-1H-indole-3-carboxylic acid;
6-chloro-5-(6-((1R,5S,6r)-6-hydroxy-3-azabicyclo[3.1.0]hexan-3-yl)-2-methoxypyridin-3-yl)-1H-indole-3-carboxylic acid;
6-chloro-5-(6-((1R,5S,6s)-6-hydroxy-3-azabicyclo[3.1.0]hexan-3-yl)-2-methoxypyridin-3-yl)-1H-indole-3-carboxylic acid; [Ex 24]
6-chloro-5-(6-(5-hydroxy-2-azaspiro[3.3]heptan-2-yl)-2-methoxypyridin-3-yl)-1H-indole-3-carboxylic acid; [Ex 25]
(R)-6-chloro-5-(6-(5-hydroxy-2-azaspiro[3.3]heptan-2-yl)-2-methoxypyridin-3-yl)-1H-indole-3-carboxylic acid;
(S)-6-chloro-5-(6-(5-hydroxy-2-azaspiro[3.3]heptan-2-yl)-2-methoxypyridin-3-yl)-1H-indole-3-carboxylic acid;
6-chloro-5-(6-(1-hydroxy-3-azabicyclo[3.1.0]hexan-3-yl)-2-methoxypyridin-3-yl)-1H-indole-3-carboxylic acid; [Ex 26]
(R)-6-chloro-5-(6-(1-hydroxy-3-azabicyclo[3.1.0]hexan-3-yl)-2-methoxypyridin-3-yl)-1H-indole-3-carboxylic acid;
(S)-6-chloro-5-(6-(1-hydroxy-3-azabicyclo[3.1.0]hexan-3-yl)-2-methoxypyridin-3-yl)-1H-indole-3-carboxylic acid;
6-chloro-5-(2-methoxy-6-(tetrahydro-1H-furo[3,4-c]pyrrol-5(3H)-yl)pyridin-3-yl)-1H-indole-3-carboxylic acid;
6-chloro-5-(2-methoxy-6-((cis)-tetrahydro-1H-furo[3,4-c]pyrrol-5(3H)-yl)pyridin-3-yl)-1H-indole-3-carboxylic acid; [Ex 27]
6-chloro-5-(6-(hexahydro-5H-furo[2,3-c]pyrrol-5-yl)-2-methoxypyridin-3-yl)-1H-indole-3-carboxylic acid;
6-chloro-5-(6-((cis)-hexahydro-5H-furo[2,3-c]pyrrol-5-yl)-2-methoxypyridin-3-yl)-1H-indole-3-carboxylic acid; [Ex 28]
6-chloro-5-(6-((3aS,6aS)-hexahydro-5H-furo[2,3-c]pyrrol-5-yl)-2-methoxypyridin-3-yl)-1H-indole-3-carboxylic acid;
6-chloro-5-(6-((3aR,6aS)-hexahydro-5H-furo[2,3-c]pyrrol-5-yl)-2-methoxypyridin-3-yl)-1H-indole-3-carboxylic acid;
6-chloro-5-(6-((3aS,6aR)-hexahydro-5H-furo[2,3-c]pyrrol-5-yl)-2-methoxypyridin-3-yl)-1H-indole-3-carboxylic acid;
5-(6-(8-oxa-3-azabicyclo[3.2.1]octan-3-yl)-2-methoxypyridin-3-yl)-6-chloro-1H-indole-3-carboxylic acid; [Ex 29]
5-(6-((1R,5S)-8-oxa-3-azabicyclo[3.2.1]octan-3-yl)-2-methoxypyridin-3-yl)-6-chloro-1H-indole-3-carboxylic acid;
5-(6-((1S,5R)-8-oxa-3-azabicyclo[3.2.1]octan-3-yl)-2-methoxypyridin-3-yl)-6-chloro-1H-indole-3-carboxylic acid;
5-(6-(2-oxa-5-azabicyclo[2.2.2]octan-5-yl)-2-methoxypyridin-3-yl)-6-chloro-1H-indole-3-carboxylic acid; [Ex 30]
5-(6-((1R,4R)-2-oxa-5-azabicyclo[2.2.2]octan-5-yl)-2-methoxypyridin-3-yl)-6-chloro-1H-indole-3-carboxylic acid;
5-(6-((1S,4S)-2-oxa-5-azabicyclo[2.2.2]octan-5-yl)-2-methoxypyridin-3-yl)-6-chloro-1H-indole-3-carboxylic acid;
6-chloro-5-(2-methoxy-6-(1-oxa-7-azaspiro[3.5]nonan-7-yl)pyridin-3-yl)-1H-indole-3-carboxylic acid; [Ex 31]
6-chloro-5-(2-methoxy-6-(tetrahydro-1H-furo[3,4-c]pyrrol-5(3H)-yl)pyridin-3-yl)-1H-indole-3-carboxylic acid;
6-chloro-5-(2-methoxy-6-((trans)-tetrahydro-1H-furo[3,4-c]pyrrol-5(3H)-yl)pyridine-3-yl)-1H-indole-3-carboxylic acid; [Ex 32]
6-chloro-5-(2-methoxy-6-((3aR, 6aR)-tetrahydro-1H-furo[3 ,4-c]pyrrol-5(3H)-yl)pyridin-3-yl)-1H-indole-3-carboxylic acid; [Ex 33]
6-chloro-5-(2-methoxy-6-((3aS, 6aS)-tetrahydro-1H-furo[3 ,4-c]pyrrol-5(3H)-yl)pyridin-3-yl)-1H-indole-3-carboxylic acid; [Ex 34]
6-chloro-5-(2-methoxy-6-[8-oxa-2-azaspiro[4.5]decan-2-yl]pyridin-3-yl)-1H-indole-3-carboxylic acid; [Ex 35]
6-chloro-5-(2-methoxy-6-(7-oxa-2-azaspiro[3.5]nonan-2-yl)pyridin-3-yl)-1H-indole-3-carboxylic acid; [Ex 36]
6-chloro-5-(2-methoxy-6-(2-oxa-7-azaspiro[4.4]nonan-7-yl)pyridin-3-yl)-1H-indole-3-carboxylic acid; [Ex 37]
5-(6-(5-acetylhexahydropyrrolo[3,4-c]pyrrol-2(1H)-yl)-2-methoxypyridin-3-yl)-6-chloro-1H-indole-3-carboxylic acid;
5-(6-((3aR, 6aS)-5-acetylhexahydropyrrolo[3,4-c]pyrrol-2(1H)-yl)-2-methoxypyridin-3-yl)-6-chloro-1H-indole-3-carboxylic acid; [Ex 38]
5-(6-((3aS, 6aR)-5-acetylhexahydropyrrolo[3,4-c]pyrrol-2(1H)-yl)-2-methoxypyridin-3-yl)-6-chloro-1H-indole-3-carboxylic acid;
5-(6-((3aR, 6aR)-5-acetylhexahydropyrrolo[3,4-c]pyrrol-2(1H)-yl)-2-methoxypyridin-3-yl)-6-chloro-1H-indole-3-carboxylic acid;
5-(6-((3aS, 6aS)-5-acetylhexahydropyrrolo[3,4-c]pyrrol-2(1H)-yl)-2-methoxypyridin-3-yl)-6-chloro-1H-indole-3-carboxylic acid;
5-(6-(6-oxa-3-azabicyclo[3.1.1]heptan-3-yl)-2-methoxypyridin-3-yl)-6-chloro-1H-indole-3-carboxylic acid; [Ex 39]
6-fluoro-5-(2-methoxy-6-[2-oxa-6-azaspiro[3.3]heptan-6-yl]pyridin-3-yl)-1H-indole-3-carboxylic acid; [Ex 40] and
4,6-difluoro-5-(2-methoxy-6-(2-oxa-6-azaspiro [3.3]heptan-6-yl)pyridin-3-yl)-1H-indole-3-carboxylic acid. [Ex 41]

57. A pharmaceutical composition comprising a compound of claim 1 and a pharmaceutically acceptable carrier.

58. A method of treating a disease or disorder associated with adenosine monophosphate-activated protein kinase (AMPK) activity in a subject, comprising administering to the subject, a therapeutically effective amount of a compound of claim 1.

59. A method of treating or preventing chronic kidney disease (CKD), end stage renal disease (ESRD), diabetic nephropathy, acute kidney injury, polycystic liver disease or polycystic kidney disease in a subject, comprising administering to the subject a therapeutically effective amount of a compound of claim 1.

60. The method of claim 59 wherein the polycystic kidney disease is autosomal dominant polycystic kidney disease (ADPKD) or autosomal recessive polycystic kidney disease (ARPKD).

61. A method of treating or preventing Type II diabetes, dyslipidemia or obesity in a subject, comprising administering to the subject a therapeutically effective amount of a compound of claim 1.

62. The method of claim 58 comprising administering to the subject a therapeutically effective amount of a second therapeutic agent.

63. The method of claim 62 wherein the second therapeutic agent is tolvaptan, vasopressin V2 receptor antagonists, glucosylceramide synthase inhibitors, nuclear factor erythroid 2-related factor 2 (NRF2) activators, cystic fibrosis transmembrane conductance regulatory (CFTR) inhibitors, angiotensin converting enzyme (ACE) inhibitors, angiotensin II receptor antagonists, sodium glucose co-transporter-2 (SGLT2) inhibitors or EGFR inhibitors.

64.-66. (canceled)
